(19) United States
(12) Patent Application Publication
(10) Pub. No.: US 2025/0059608 A1
(43) Pub. Date: Feb. 20, 2025

(54) MOLECULAR SIGNATURES FOR CELL TYPING AND MONITORING IMMUNE HEALTH

(71) Applicant: ALLEN INSTITUTE, Seattle, WA (US)

(72) Inventors: Thomas F. BUMOL, Seattle, WA (US); Xiao-Jun LI, Seattle, WA (US); Adam SAVAGE, Seattle, WA (US); Peter SKENE, Seattle, WA (US); Troy TORGERSON, Seattle, WA (US); Suhas VASAIKAR, Seattle, WA (US)

(73) Assignee: ALLEN INSTITUTE, Seattle, WA (US)

(21) Appl. No.: 18/721,154

(22) PCT Filed: Dec. 19, 2022
(86) PCT No.: PCT/US22/81977

§ 371 Date: Jun. 17, 2024

Related U.S. Application Data
(60) Provisional application No. 63/291,234, filed on Dec. 17, 2021.

(51) Int. Cl. C12Q 1/6886; C12Q 1/6881; C12Q 1/70; G16B 20/20

(52) U.S. Cl. CPC C12Q 1/6886; C12Q 1/6881; C12Q 1/70; G16B 20/20; C12Q 2600/158

(57) ABSTRACT

Provided herein are methods, devices, and systems comprising identification of a panel of gene markers useful for immune cell typing and identifying, detecting, and/or monitoring disease conditions in a subject in need thereof, including infections, cancer and autoimmune conditions. The methods also provide for assays for determining the levels of said markers prior to and after treatment of a condition in the subject, wherein the condition is impacted by one or more factors including age, occupation or environment of the subject.

### CROSS-REFERENCE TO RELATED APPLICATION(S)

This application claims the benefit of U.S. Provisional Patent Application No. 63/291,234, filed on Dec. 17, 2021, the contents of which are incorporated by reference in their entirety.

### BACKGROUND

Applying multi-omics technologies to measure longitudinal specimens of human participants provides unprecedented insights on disease such as COVID-19, diabetes, and lymphoma. Single-cell technologies, such as single-cell ribonucleic acid sequencing (scRNA-seq) and single-cell assay for transposase-accessible chromatin sequencing (scATAC-seq), can offer granular details on disease mechanisms and are increasingly utilized in biological and clinical research. It is anticipated that more and more longitudinal bulk and single-cell omics data will be generated by the scientific community.

Different statistical methods are used to analyze longitudinal data to account for the diversities in research interest, study design, and/or data type (continuous or categorical). Generalized linear mixed model (GLMM) is a popular approach for analyzing continuous longitudinal data. It is common that the same dataset can be examined from multiple perspectives with different methods. Complications such as human heterogeneity, interdependency between multiple samples of same participant, missing and/or incomplete data, unbalanced dataset, and unexpected outlier events (e.g., severe adverse events in clinical trials) are all intrinsic to longitudinal data. The usage of single-cell technologies brings additional complications, such as dropout, sparseness, interdependency between cells of same sample, and unbalanced cell counts in individual samples. Advanced methods have been applied to analyze longitudinal bulk omics data with customized codes for specific projects. Sophisticated methods for analyzing cross-sectional single-cell omics data have also been developed with mixed performance. However, it is challenging to analyze longitudinal bulk and single-cell omics data. Instead, researchers rely on customized codes to analyze such data, which is time-consuming, error-prone, and a non-small challenge to many people. A comprehensive yet simple-to-use analysis platform to extract insightful information from longitudinal omics data is desired.

### SUMMARY

Provided herein are methods, devices, and systems comprising a gene panel useful for immune cell typing and identifying, detecting, and/or monitoring disease conditions in a subject in need thereof.

In some aspects, provided is a method of identifying, detecting, and/or monitoring a health condition in a subject in need thereof, comprising measuring levels of a set of genes in a biological sample obtained from the subject, wherein the set of genes comprises all or a subset of A1BG, ABLIM1, AC020656.1, AC243960.1, ADTRP, AFF3, ALDH2, ANXA2R, APOBEC3C, APP, AQP3, ARID5B, ATF7IP2, BANK1, BCL11A, BCL11B, BIRC3, BLK, CAMK4, CAPG, CARS, CASP8AP2, CBL, CCDC167, CCDC50, CCL4, CCND2, CCR7, CD14, CD27, CD36, CD6, CD68, CD79A, CD79B, CD8A, CD8B, CD96, CDKN1C, CEBPD, CFD, CFP, CLEC10A, CLEC12A, CLIC3, CMC1, CPVL, CSF3R, CST7, CSTA, CTSH, CXXC5, CYBB, CYTOR, DCTPP1, DNAJB1, DOK2, DYNLL2, DYRK2, EAF2, EBP, ERN1, FCER1A, FCER1G, FCER2, FCGR3A, FCN1, FCRL1, FGFBP2, FGL2, FHIT, FKBP11, GATA3, GBP5, GIMAP7, GNG2, GPR65, GRN, GZMA, GZMB, GZMH, GZMK, HLA-DMA, HLA-DMB, HLA-DQA1, HOPX, IFITM3, IFT57, IGHD, IGHM, IGLC2, IGSF6, IKZF3, IL2RB, IL3RA, IL4R, IL6ST, INPP4B, IRF7, IRF8, ITGAL, ITM2C, JAML, JCHAIN, JUN, KLRB1, KLRC1, KLRD1, KLRF1, KLRG1, LEF1, LGALS2, LGALS3, LILRA4, LIN000623, LIN000861, LIN000926, LINC01857, LINC01871, LINC02446, LRRC25, LY86, LYN, LYST, MAL, MAML2, MAPKAPK2, MARCHF1, MARCKS, MATK, MEF2C, MHENCR, MNDA, MS4A1, MS4A6A, MS4A7, MT1X, MYBL1, MYC, MYO1F, MZB1, NCF2, NCR3, NELL2, ORAI2, OXNAD1, PAG1, PASK, PDE3B, PDLIM1, PECAM1, PHACTR2, PIK3IP1, PILRA, PITPNC1, PLD4, PLPP5, POU2AF1, POU2F2, PPP1R10, PRF1, PRKCH, PRR5, PTPN4, PTPN6, PYHIN1, RALGPS2, RASSF1, RCAN3, RFLNB, RHOC, RNF130, RTKN2, S100A12, S100B, SAMD3, SELENOM, SERPINA1, SERPINF1, SESN3, SLC2A4RG, SLC4A10, SMIM25, SP140, SPI1, SPIB, SPON2, STMN1, STMN3, STX7, SWAP70, SYNE1, TAGAP, TBC1 D15, TC2N, TCF4, TCL1A, THEM4, TMEM154, TMEM156, TMIGD2, TNFRSF13C, TNFRSF1B, TPD52, TPM2, TPST2, TRABD2A, TRDC, TRG-AS1, TRGC1, TRGC2, TSHZ2, TSPAN3, TULP4, UGCG, VCAN, XCL1, XCL2, ZAP70, and ZEB2.

In some embodiments, the health condition is a condition impacted by age, environmental, occupational, and/or physical factors.

In some embodiments, the health condition is a disease condition. In some embodiments, the method further comprises treating the subject for the disease condition. In some embodiments, the method further comprises measuring levels of the set of genes in a second biological sample obtained from the subject after the treatment.

In some embodiments, the set of genes comprises about 10 or more genes, about 25 or more genes, about 50 or more genes, about 100 or more genes, about 150 or more genes, or about 200 or more genes.

In some embodiments, the biological sample is a tissue sample. In some embodiments, the biological sample is a blood sample. In some embodiments, the biological sample comprises peripheral blood mononuclear cells (PBMCs). In some embodiments, the biological sample comprises circulating tumor cells (CTCs).

In some embodiments, the measuring step is carried out by single cell technology. In some embodiments, the single cell technology comprises single-cell ribonucleic acid sequencing (scRNA-seq) and/or single-cell assay for transposase-accessible chromatin sequencing (scATAC-seq).

In some embodiments, the disease condition is a viral infection, for example, influenza or SARS-CoV-2 infection.

In some embodiments, the disease condition is cancer. In some embodiments, the cancer is a hematological malignancy, for example, monoclonal B cell lymphocytosis, multiple myeloma, myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), blast crisis chronic myelogenous leukemia (bcCML), B cell acute lymphoid leukemia (B-ALL), T cell acute lymphoid leukemia (T-ALL), T cell lymphoma, and B cell lymphoma. In some embodiments, the cancer is a solid tumor, for example, lung cancer, breast cancer, liver cancer, stomach cancer, colon cancer, rectal cancer, kidney cancer, gastric cancer, gallbladder cancer, cancer of the small intestine, esophageal cancer, melanoma, bone cancer, pancreatic cancer, skin cancer, uterine cancer, ovarian cancer, testicular cancer, cancer of the thyroid gland, cancer of the adrenal gland, bladder cancer, and glioma.

In some embodiments, the disease condition is an autoimmune disease, for example, type 1 diabetes, lupus, systemic lupus erythematosus, rheumatoid arthritis, psoriasis, psoriatic arthritis, multiple sclerosis, inflammatory bowel disease, Crohn's disease, ulcerative colitis, Addison's disease, Graves' disease, Sjögren's syndrome, Hashimoto's thyroiditis, myasthenia gravis, autoimmune vasculitis, pernicious anemia, and celiac disease.

In some aspects, provided is a method of identifying, labeling, and/or quantifying immune cell types in a biological sample, comprising measuring levels of a set of genes in the biological sample, wherein the set of genes comprises all or a subset of A1BG, ABLIM1, AC020656.1, AC243960.1, ADTRP, AFF3, ALDH2, ANXA2R, APOBEC3C, APP, AQP3, ARID5B, ATF7IP2, BANK1, BCL11A, BCL11B, BIRC3, BLK, CAMK4, CAPG, CARS, CASP8AP2, CBL, CCDC167, CCDC50, CCL4, CCND2, CCR7, CD14, CD27, CD36, CD6, CD68, CD79A, CD79B, CD8A, CD8B, CD96, CDKN1C, CEBPD, CFD, CFP, CLEC10A, CLEC12A, CLIC3, CMC1, CPVL, CSF3R, CST7, CSTA, CTSH, CXXC5, CYBB, CYTOR, DCTPP1, DNAJB1, DOK2, DYNLL2, DYRK2, EAF2, EBP, ERN1, FCER1A, FCER1G, FCER2, FCGR3A, FCN1, FCRL1, FGFBP2, FGL2, FHIT, FKBP11, GATA3, GBP5, GIMAP7, GNG2, GPR65, GRN, GZMA, GZMB, GZMH, GZMK, HLA-DMA, HLA-DMB, HLA-DQA1, HOPX, IFITM3, IFT57, IGHD, IGHM, IGLC2, IGSF6, IKZF3, IL2RB, IL3RA, IL4R, IL6ST, INPP4B, IRF7, IRF8, ITGAL, ITM2C, JAML, JCHAIN, JUN, KLRB1, KLRC1, KLRD1, KLRF1, KLRG1, LEF1, LGALS2, LGALS3, LILRA4, LINC00623, LINC00861, LINC00926, LINC01857, LINC01871, LINC02446, LRRC25, LY86, LYN, LYST, MAL, MAML2, MAPKAPK2, MARCHF1, MARCKS, MATK, MEF2C, MHENCR, MNDA, MS4A1, MS4A6A, MS4A7, MT1X, MYBL1, MYC, MYO1F, MZB1, NCF2, NCR3, NELL2, ORAI2, OXNAD1, PAG1, PASK, PDE3B, PDLIM1, PECAM1, PHACTR2, PIK3IP1, PILRA, PITPNC1, PLD4, PLPP5, POU2AF1, POU2F2, PPP1 R10, PRF1, PRKCH, PRR5, PTPN4, PTPN6, PYHIN1, RALGPS2, RASSF1, RCAN3, RFLNB, RHOC, RNF130, RTKN2, S100A12, S100B, SAMD3, SELENOM, SERPINA1, SERPINF1, SESN3, SLC2A4RG, SLC4A10, SMIM25, SP140, SPI1, SPIB, SPON2, STMN1, STMN3, STX7, SWAP70, SYNE1, TAGAP, TBC1D15, TC2N, TCF4, TCL1A, THEM4, TMEM154, TMEM156, TMIGD2, TNFRSF13C, TNFRSF1B, TPD52, TPM2, TPST2, TRABD2A, TRDC, TRG-AS1, TRGC1, TRGC2, TSHZ2, TSPAN3, TULP4, UGCG, VCAN, XCL1, XCL2, ZAP70, and ZEB2.

In some embodiments, the immune cell types comprise normal immune cells and abnormal immune cells. In some embodiments, the immune cell types comprise B cells, T cells, natural killer (NK) cells, monocytes, macrophages, dendritic cells (DCs), mast cells, neutrophils, eosinophils, and basophils.

In some aspects, provided is a single cell assay kit comprising probes for measuring levels of a set of genes in a biological sample, wherein the set of genes comprises all or a subset of A1BG, ABLIM1, AC020656.1, AC243960.1, ADTRP, AFF3, ALDH2, ANXA2R, APOBEC3C, APP, AQP3, ARID5B, ATF7IP2, BANK1, BCL11A, BCL11B, BIRC3, BLK, CAMK4, CAPG, CARS, CASP8AP2, CBL, CCDC167, CCDC50, CCL4, CCND2, CCR7, CD14, CD27, CD36, CD6, CD68, CD79A, CD79B, CD8A, CD8B, CD96, CDKN1C, CEBPD, CFD, CFP, CLEC10A, CLEC12A, CLIC3, CMC1, CPVL, CSF3R, CST7, CSTA, CTSH, CXXC5, CYBB, CYTOR, DCTPP1, DNAJB1, DOK2, DYNLL2, DYRK2, EAF2, EBP, ERN1, FCER1A, FCER1G, FCER2, FCGR3A, FCN1, FCRL1, FGFBP2, FGL2, FHIT, FKBP11, GATA3, GBP5, GIMAP7, GNG2, GPR65, GRN, GZMA, GZMB, GZMH, GZMK, HLA-DMA, HLA-DMB, HLA-DQA1, HOPX, IFITM3, IFT57, IGHD, IGHM, IGLC2, IGSF6, IKZF3, IL2RB, IL3RA, IL4R, IL6ST, INPP4B, IRF7, IRF8, ITGAL, ITM2C, JAML, JCHAIN, JUN, KLRB1, KLRC1, KLRD1, KLRF1, KLRG1, LEF1, LGALS2, LGALS3, LILRA4, LIN000623, LIN000861, LIN000926, LINC01857, LINC01871, LINC02446, LRRC25, LY86, LYN, LYST, MAL, MAML2, MAPKAPK2, MARCHF1, MARCKS, MATK, MEF2C, MHENCR, MNDA, MS4A1, MS4A6A, MS4A7, MT1X, MYBL1, MYC, MYO1F, MZB1, NCF2, NCR3, NELL2, ORAI2, OXNAD1, PAG1, PASK, PDE3B, PDLIM1, PECAM1, PHACTR2, PIK3IP1, PILRA, PITPNC1, PLD4, PLPP5, POU2AF1, POU2F2, PPP1R10, PRF1, PRKCH, PRR5, PTPN4, PTPN6, PYHIN1, RALGPS2, RASSF1, RCAN3, RFLNB, RHOC, RNF130, RTKN2, S100A12, S100B, SAMD3, SELENOM, SERPINA1, SERPINF1, SESN3, SLC2A4RG, SLC4A10, SMIM25, SP140, SPI1, SPIB, SPON2, STMN1, STMN3, STX7, SWAP70, SYNE1, TAGAP, TBC1D15, TC2N, TCF4, TCL1A, THEM4, TMEM154, TMEM156, TMIGD2, TNFRSF13C, TNFRSF1B, TPD52, TPM2, TPST2, TRABD2A, TRDC, TRG-AS1, TRGC1, TRGC2, TSHZ2, TSPAN3, TULP4, UGCG, VCAN, XCL1, XCL2, ZAP70, and ZEB2.

### BRIEF DESCRIPTION OF THE DRAWINGS

FIGS. 1A-1H: General workflow and analysis schema of the platform for analyzing longitudinal multi-omics (PALMO) data. FIG. 1A: PALMO can work with complex longitudinal data, including clinical data, bulk omics data, and single-cell omics data. FIG. 1B: Overview of five analytical modules implemented in PALMO. FIG. 1C: Variance decomposition analysis (VDA) applies generalized linear mixed model to assess contributions of factors of interest (such as disease status, sex, individual participant, cell type, experimental batch, etc.) to the total variance of individual features in the data. FIG. 1D: Coefficient of variation (CV) profiling (CVP) is designed for bulk longitudinal data, calculates CV of repeated measurements on the same participant to assess the corresponding longitudinal stability, and compares CVs of different participants to identify consistently stable or variable features. FIG. 1E: Stability pattern evaluation across cell types (SPECT) is the CVP counterpart for single-cell omics data, analyzes stability patterns of features across different cell types and different participants, classifies features based on how often they are stable or variable in cell type-donor combinations, and identifies features that are unique to individual cell types and consistent among participants. FIG. 1F: Outlier detection analysis (ODA) evaluates how many features in a sample are outliers when compared with the corresponding features in other samples of same participants, assesses whether the number of outlier features in the sample is significantly higher than expectation, and identifies possible abnormal events occurred during a longitudinal study. FIG. 1G: Time course analysis (TCA) uses the hurdle model to evaluate transcriptomic changes over time based on longitudinal scRNA-seq data of same participants, models time as a continuous variable for data with at least three timepoints, and identifies up- or down-regulated genes over time. FIG. 1H: PALMO uses circos plots to display CVs of features of interest and reveal stability patterns across features, participants, cell types, and data modalities.

FIGS. 2A-2H: Variance decomposition on longitudinal single-cell omics data. FIG. 2A: Overall distributions of variance explained by inter-donor variations (Donor), longitudinal intra-donor variations (Week), variations among cell types (Celltype), or residual variations (Residual) based on scRNA-seq data. FIGS. 2B and 2C: Examples of genes whose total expression variance was most explained by inter-cell-type variations (FIG. 2B) or inter-donor variations (FIG. 2C). FIG. 2D: Examples of genes that had the most but still minuscular intra-donor variations in expression. FIG. 2E: Same as FIG. 2A but based on scATAC-seq data. FIGS. 2F and 2G: The top list of genes whose inter-cell-type (FIG. 2F) or inter-donor (FIG. 2G) variations contributed most to the total variance in scATAC-seq data. FIG. 2H: The top list of genes that had the most intra-donor variations in scATAC-seq data. In FIGS. 2B-2D, Kruskal-Wallis test was used to calculate the p value. ICC: intra-class correlation.

FIGS. 3A-3E: Longitudinal stability of plasma proteome. FIG. 3A: Scatter plots of coefficient of variation (CV) versus mean of normalized protein expression (NPX) over timepoints in six donors. The longitudinal stable and variable proteins are represented in blue and red, respectively. FIGS. 3B and 3C: Heatmap of CV of top 50 longitudinally variable (FIG. 3B CV>5%) or stable (FIG. 3C CV<5%) plasma proteins. FIG. 3D: Top panel: Number of proteins with z>2.5 (red) or z<−2.5 (blue) in individual samples, where z=(NPX−NPX)/SD with NPX and SD being the mean and the standard deviation, respectively, of NPX across samples of the same participant. Bottom panel: −log₁₀ (p_{adj}) for individual samples being possible outliers, where p_{adj} is calculated based on a binomial test and adjusted by Benjamini and Hochberg procedure for p-values of all samples. FIG. 3E: Protein examples clearly demonstrate that Week 6 of donor PTID3 was an outlier.

FIGS. 4A-4I: Properties of 220 STATIC genes of peripheral blood mononuclear cells (PBMCs). FIG. 4A: Heatmap of coefficient of variation (CV) evaluated on 93 out of the 220 stable across time in cell-types (STATIC) genes that were identified from nineteen cell types in the longitudinal scRNA-seq data of four healthy donors. The 93 STATIC genes include up to ten top STATIC genes from individual cell types. FIG. 4B: Circos plots displaying CV of five example STATIC genes identified from each of five major cell types: T cells, B cells, natural killer (NK) cells, monocytes, and dendritic cells (DCs). FIG. 4C: Uniform Manifold Approximation and Projection (UMAP) using only the 220 STATIC genes as input features (sUMAP) on the same longitudinal scRNA-seq data. FIGS. 4D-4F: sUMAP using the same 220 STATIC genes on three external PBMC datasets (FIG. 4D, Zhu et al., 2020 (CNP0001102); FIG. 4E, Lee et al., 2020 (GSE149689); FIG. 4F, Hao et al., 2021 (GSE164378)), where cells are labeled as in the original studies. FIG. 4G: Distributions of Pearson correlation coefficient between gene expression in scRNA-seq data and gene score in scATAC-seq data, one for the 220 STATIC genes (median correlation 0.70), one for the top 250 highly variable genes (HVGs, median correlation 0.37), one for the 10,611 reliable genes (average expression 20.1, median correlation 0.21), and one for random gene pairs (95% upper confidence bound at 0.399). FIGS. 4H and 4I: Venn diagrams showing the overlaps between the 220 STATIC genes and biomarkers distinguishing either healthy controls (Normal) versus participants infected with influenza (FLU, left panel) or Normal versus participants infected with SARS-CoV-2 (COVID-19, right panel). The biomarkers were identified from the dataset in either FIG. 4H, Zhu et al., 2020 (CNP0001102) or FIG. 4I, Lee et al., 2020 (GSE149689).

FIGS. 5A-5D: Properties of 304 STATIC genes of mouse brain tissue. FIG. 5A: Heatmap of coefficient of variation (CV) of the 304 STATIC genes that were identified from 25 cell types in the scRNA-seq data of a mouse brain study (Ximerakis et al., 2019; GSE129788). FIG. 5B: UMAP using only the 304 STATIC genes as input features (sUMAP) on the same scRNA-seq data. Cells are labeled as in the original study. FIG. 5C: Percentage of top STATIC genes that overlap with cell-type marker genes identified in the original study. Up to 25 top STATIC genes from each cell type are compared with the corresponding marker genes of the same cell type. FIG. 5D: Venn diagram showing the overlap between the 234 STATIC genes identified from 15 out of the 25 cell types and biomarkers distinguishing young versus old mice that were identified in the original study from the same 15 cell types.

FIGS. 6A-6F: Circos plots showing stability patterns of five protein families. FIG. 6A: Circos plot displaying stability patterns of gene expression (outer circles) and gene score (inner circles) of human leukocyte antigen (HLA) protein family (member: HLA-A, HLA-B, HLA-C, HLA-DRA, HLA-DPA1, and HLA-DRB1). Samples with missing data or cell types with low cell counts are shown in grey. FIGS. 6B-6F: Same as FIG. 6A, but for FIG. 6B, interferon regulatory factors (IRFs; member: IRF1, IRF2, IRF3, IRF4, IRF5, and IRF8), FIG. 6C, interleukins (ILs; member: IL32, IL7R, IL10RA, IL2RB, IL1B, and IL18), FIG. 6D, chemokine (C-X-C motif) receptor/ligand (CXCR/L) protein family (member: CXCR4, CXCR5, CXCR6, CSCL8, CSCL10, and CSCL16), FIG. 6E, Janus kinase (JAK) and signal transducer and activator of transcription (STAT) protein family (member: JAK1, JAK2, JAK3, STAT3, STAT4, and STAT6), and FIG. 6F, tumor necrosis factor receptor superfamily (TNFRSF; member: TNFRSF1B, TNFRSF13C, TNFRSF10B, TNFRSF25, TNFRSF11A, and TNFRSF17).

FIGS. 7A-7E: Heterogeneous immune responses by COVID-19 patients during recovery. FIG. 7A: Volcano plot showing temporal expression changes of individual genes in different cell types during the recovery of patient COV-3 (female, 41 years old, mild symptoms, data on day D1/D4/D16), based on longitudinal scRNA-seq data in Zhu et al., 2020 (CNP0001102). The x-axis shows the slope (coefficient) of gene expression change as a linear function of time. The y-axis shows the corresponding adjusted p value of the slope. FIGS. 7B-7D: Same as FIG. 7A, but for patients COV-2 (FIG. 7B; male, 45 years old, mild symptoms, data on D1/D4/D7/D10/D16), COV-1 (FIG. 7C; male, 15 years old, mild symptoms, data on D1/D4/D16), and COV-5 (FIG. 70; female, 85 years old, severe symptoms, data on D1/D7/D13). FIG. 7E: Counts of significantly upregulated (adjusted p<0.05 and slope>0.1, red) and significantly downregulated (adjusted p<0.05 and slope <0.1, blue) genes during the recovery of the four COVID-19 patients in individual cell types.

FIG. 8: Flow cytometry gating schemes. Red labels indicate gates used to determine population frequencies.

FIGS. 9A-9E: Longitudinal scRNA-seq data and scATAC-seq data on PBMCs of four healthy participants over six weeks. FIG. 9A: UMAP of scRNA-seq data consisting of 472,464 PBMCs. The dot color represents identified cell types based on Seurat V2. FIG. 9B: Distributions of labeling scores of individual cell types as observed in scRNA-seq data. Cells having scores below the red vertical dashed lines (0.5) were filtered out from analysis due to poor labeling quality. FIG. 9C: Pearson correlations between frequencies of the same cell types as measured by scRNA-seq or flow cytometry on all samples. FIG. 9D: UMAP projection of scATAC-seq data using iterative latent semantic indexing (LSI) for clustering and Seurat algorithm for cell labeling, as implemented in ArchR. FIG. 9E: Distributions of labeling scores of individual cell types as observed in scATAC-seq data. Cells having scores below the red vertical dashed lines (0.5) were filtered out from analysis due to poor labeling quality.

FIGS. 10A-10F: Variance decomposition on bulk longitudinal data. FIG. 10A: Overall distributions of total variance explained by inter-donor variations (Donor), longitudinal intra-donor variations (Week) or residual variations (Residual) based on complete blood count (CBC) data as measured on six healthy participants over ten weeks. FIG. 10B: Variance of specific CBC measurements that was explained by Donor, Week, or Residual. FIG. 10C: Overall distributions of total variance explained by Donor, Week, or Residual based on peripheral blood mononuclear cell (PBMC) frequencies as measured by flow cytometry on four healthy participants over six weeks. FIG. 10D: Variance of specific PBMC frequencies that was explained by Donor, Week, or Residual. FIG. 10E: Overall distributions of total variance explained by Donor, Week, or Residual based on plasma protein abundance as measured on six healthy participants over ten weeks. FIG. 10F: Examples of proteins whose total variance was most explained by inter-donor variations (top panel) or intra-donor variations (bottom panel).

FIGS. 11A and 11B: Comparison between variance decomposition analysis (VDA) and variancePartition. FIG. 11A: Scatter plots of percentage of total variance explained by donor (left panel), tissue (middle panel), or batch (right panel) as obtained by using VDA or variancePartition. The simulated dataset of 200 genes in 100 samples of 25 donors is described in “Tutorial on using variancePartition” at https-/bioconductor.org/packages/release/bioc/html/variancePartition.html (accessed on Sep. 9, 2022). FIG. 11B: Scatter plots of percentage of total variance explained by donor (left panel) or time (right panel) as obtained by using VDA or variancePartition on our longitudinal proteomics data after removing 922 proteins with missing values.

FIGS. 12A-12H: Variance decomposition on T cell receptor (TCR) sequencing data. FIG. 12A: Overall distributions of total variance explained by inter-donor variations (Donor), longitudinal intra-donor variations (Time), inter-subtype variations (Subtype), or residual variations (Residual) based on sequencing data of TCR 1-chains from sorted CD4+ T cells of four systemic sclerosis (SSc) donors, each contributing three samples over more than two years. The two SSc subtypes considered are limited SSc and diffuse SSc. FIGS. 12B-12D: Examples of clonotypes showing most inter-donor variations (FIG. 12B), intra-donor variations (FIG. 12C), or inter-subtype variations (FIG. 12D). FIG. 12E: Same as FIG. 12C but for TCRβ data of the corresponding CD8+ T cells. FIGS. 12F-12H: Same as FIGS. 12B-12D but for TCRβ data of the corresponding CD8+ T cells.

FIGS. 13A-13D: Coefficient of variation (CV) profiling (CVP) of longitudinal plasma proteomics data. FIG. 13A: Histogram of coefficient of variation (CV) of normalized protein expression (NPX) over timepoints in six donors. CV of 5% was selected as the cutoff separating longitudinally stable versus variable proteins. FIG. 13B: Heatmap showing NPX intra- and inter-donor correlations. FIG. 13C: Top pathways (p<0.05) from gene set enrichment analysis (GSEA) on outlier proteins detected in donor PTID3 at week 6. FIG. 13D: Single-sample GSEA (ssGSEA) on outlier proteins, showing enrichment in MYC targets, IFN-alpha response at week 6.

FIG. 14: Scatter plots of coefficient of variation (CV) of longitudinal scRNA-seq data of individual cell types. Scatter plots of CV versus mean of gene expression (log 2(avg counts)) over timepoints of individual donors. Only reliable genes with an average expression ≥0.1 were kept. Results from individual donors were calculated separately and combined. Housekeeping genes ACTB and GAPDH (blue) were used to select a CV threshold of 10% by which genes were split into longitudinally stable (red) or variable (black).

FIGS. 15A-15C: Longitudinally variable and stable genes across nineteen cell types. FIG. 15A: Heatmap of coefficient of variation (CV) of the top 25 super variable (SUV) genes. FIG. 15B: Heatmap of CV of the top 25 super stable (SUS) genes. CVs of the housekeeping genes ACTB and GAPDH are also shown for comparison. FIG. 15C: Venn diagram showing overlaps between SUV genes, stable across time in cell-types (STATIC) genes, variable across time in cell-types (VATIC) genes, and SUS genes.

FIGS. 16A-16J: The five most correlated genes between expression in scRNA-seq data and gene score in scATAC-seq data. FIGS. 16A-16E: Scatter plots between expression in scRNA-seq data and gene score in scATAC-seq data of the five most correlated genes (LEF1, TNFRSF13C, CST7, SPI1, and SERPINF1). FIGS. 16F-16J: Open chromatin regions around the five most correlated genes in different cell types using ArchR visualization of scATAC-seq data.

FIGS. 17A-17F: Correlations of six protein families between expression in scRNA-seq data and gene score in scATAC-seq data. FIG. 17A: Human leukocyte antigens (HLAs). FIG. 17B: Interferon regulatory factors (IRFs). FIG. 17C: Interleukins (ILs). FIG. 17D: chemokine (C-X-C motif) receptor/ligand (CXCR/L) family. FIG. 17E: Janus kinases (JAKs) and signal transducer and activator of transcription proteins (STATs). FIG. 17F: Tumor necrosis factor receptor superfamily (TNFRSF).

FIGS. 18A-18D: Comparison between time course analysis (TCA) and Seurat on longitudinal scRNA-seq data of a COVID-19 patient (COV-5). FIG. 18A: Venn diagram for differential expression genes (DEGs) from TCA and DEGs from two runs of Seurat analyses: D1 versus D7+D13 or D1+D7 versus D13. FIGS. 18B-18D: Top 10 up- and top 10 down-regulated genes from Seurat D1 versus D7+D13 analysis (FIG. 18B), Seurat D1+D7 versus D13 analysis (FIG. 18C), and TCA (FIG. 18D).

FIG. 19: Flow-gating strategy to identify B abnormal cells population from peripheral blood mononuclear cells (PBMC).

FIG. 20: Examples showing (a) abnormal and (b) normal B cell populations.

FIGS. 21A-21B: Observed B cell populations on study participants. FIG. 21A: The panel shows 12 healthy donors (9 males and 3 females) with normal B cell populations. FIG. 21B: The panel shows 4 donors with abnormal mature memory B cells (highlighted in dashed line).

FIGS. 22A-22I: Uniform Manifold Approximation and Projection (UMAP) of scRNA-seq data consisting of 80,000 PBMCs. FIG. 22A: Highly variable genes (HVGs, n=3000) were used to cluster and visualize cells in UMAP. FIG. 22B: B cells were first isolated and then clustered and visualized in UMAP using HVGs. FIG. 22C: Distribution of B cells from the 16 participants. FIGS. 22D-22F: Same as FIGS. 22A-22C, based on the STATIC 220 genes instead of the 3000 HVGs. FIGS. 22G-22I: Same as FIGS. 22A-22C, based on the 500 genes instead of the 3000 HVGs. For FIGS. 22A, 22D, and 22G, the same Seurat V2 labeling was used to annotate cells. For FIGS. 22B, 22C, 22E, 22F, 22H, and 22I, abnormal B cells were highlighted in a dashed line.

FIGS. 23A-23B: B cell UMAP density plots comparing B cells of healthy controls and those of likely monoclonal B lymphocytosis (MBL) participants. B cell UMAP density plots of a healthy young adult (BR1), a healthy older adult (BR2), two likely MBL participants from Benaroya Research Institute (BR2049, BR2007), and two likely MBL participants from Colorado University (CU1022 and CU1049) using the STATIC 220 genes (FIG. 23A) or the 500 gene list (FIG. 23B) are shown.

FIG. 24: Gene ontology enrichment for the STATIC 220 genes and 500 genes.

FIGS. 25A-25B: Comparison between Seurat based label transfer and that using only the STATIC 220 genes (FIG. 25A) or the 500 genes (FIG. 25B).

FIGS. 26A-26B: The overall classification of clustering accuracy of k-nearest neighbors (KNN) model based on the training dataset and UMAP visualizations on the training and the projected testing dataset. FIG. 26A: The average accuracy of 5-fold cross validations on the training dataset. FIG. 26B: UMAP plot of the training and testing datasets colored by cell type and clusters. The cell type labels are inferred from Seurat V4 based on the 220 STATIC genes only.

FIG. 27: The boxplot of centered log ratio (CLR) transformed frequency for cluster 5 and 7. Each dot represents a single sample in the corresponding cohort group. The p-values are calculated based on the Wilcoxon test. FH1 cohorts_group contains FH1_PreTreatment and FH1_Post_Induction. The rest of cohorts are in other cohorts_group.

FIG. 28A: Spatial distribution of POU2AF1, one of the STATIC 220 genes, shows defined tissue domain specific distribution. The number of detected POU2AF1 transcripts per cell is log-transformed and mapped to a color gradient which ranges from blue (low levels of detection) to red (high levels of detection). FIG. 28B: Cells in the tonsil cross-section are projected into the UMAP space (left panel). Each point represents a cell, and the color indicates the cluster membership of the cell by Leiden clustering. Cells in the geometric space defined by the microscopy field color coded by their Leiden cluster membership (right panel).

FIG. 29: Comparison of the average number of UMIs per cell for each gene in the STATIC 220 panel, normalized for UMI depth for each chemistry.

FIG. 30: Confusion matrix comparing cell type labels using either full Fixed RNA Profiling (FRP) panel or the STATIC 220 panel for Level 1 and Level 2.

FIG. 31: Comparing the number of DEGs captured by each chemistry as it relates to the size of the panel used.

### DETAILED DESCRIPTION

While the present disclosure is capable of being embodied in various forms, the description below of several embodiments is made with the understanding that the present disclosure is to be considered as an exemplification of the invention and is not intended to limit the invention to the specific embodiments illustrated.

Headings are provided for convenience only and are not to be construed to limit the invention in any manner. Embodiments illustrated under any heading may be combined with embodiments illustrated under any other heading.

In some aspects, provided is a set of genes associated with an immune response. The presence, absence, and/or level of the set of genes may function as a molecular immune signature that can be used in methods, devices, and/or systems for immune cell typing and identifying, detecting, and/or treating disease conditions associated with an immune response, according to some embodiments.

In some embodiments, the set of genes includes all or a subset of the following genes (also referred to as the STATIC 220 genes in certain embodiments): A1BG, ABLIM1, AC020656.1, AC243960.1, ADTRP, AFF3, ALDH2, ANXA2R, APOBEC3C, APP, AQP3, ARID5B, ATF7IP2, BANK1, BCL11A, BCL11B, BIRC3, BLK, CAMK4, CAPG, CARS, CASP8AP2, CBL, CCDC167, CCDC50, CCL4, CCND2, CCR7, CD14, CD27, CD36, CD6, CD68, CD79A, CD79B, CD8A, CD8B, CD96, CDKN1C, CEBPD, CFD, CFP, CLEC10A, CLEC12A, CLIC3, CMC1, CPVL, CSF3R, CST7, CSTA, CTSH, CXXC5, CYBB, CYTOR, DCTPP1, DNAJB1, DOK2, DYNLL2, DYRK2, EAF2, EBP, ERN1, FCER1A, FCER1G, FCER2, FCGR3A, FCN1, FCRL1, FGFBP2, FGL2, FHIT, FKBP11, GATA3, GBP5, GIMAP7, GNG2, GPR65, GRN, GZMA, GZMB, GZMH, GZMK, HLA-DMA, HLA-DMB, HLA-DQA1, HOPX, IFITM3, IFT57, IGHD, IGHM, IGLC2, IGSF6, IKZF3, IL2RB, IL3RA, IL4R, IL6ST, INPP4B, IRF7, IRF8, ITGAL, ITM2C, JAML, JCHAIN, JUN, KLRB1, KLRC1, KLRD1, KLRF1, KLRG1, LEF1, LGALS2, LGALS3, LILRA4, LINC00623, LINC00861, LINC00926, LINC01857, LINC01871, LINC02446, LRRC25, LY86, LYN, LYST, MAL, MAML2, MAPKAPK2, MARCHF1, MARCKS, MATK, MEF2C, MHENCR, MNDA, MS4A1, MS4A6A, MS4A7, MT1X, MYBL1, MYC, MYO1F, MZB1, NCF2, NCR3, NELL2, ORAI2, OXNAD1, PAG1, PASK, PDE3B, PDLIM1, PECAM1, PHACTR2, PIK3IP1, PILRA, PITPNC1, PLD4, PLPP5, POU2AF1, POU2F2, PPP1 R10, PRF1, PRKCH, PRR5, PTPN4, PTPN6, PYHIN1, RALGPS2, RASSF1, RCAN3, RFLNB, RHOC, RNF130, RTKN2, S100A12, S100B, SAMD3, SELENOM, SERPINA1, SERPINF1, SESN3, SLC2A4RG, SLC4A10, SMIM25, SP140, SPI1, SPIB, SPON2, STMN1, STMN3, STX7, SWAP70, SYNE1, TAGAP, TBC1D15, TC2N, TCF4, TCL1A, THEM4, TMEM154, TMEM156, TMIGD2, TNFRSF13C, TNFRSF1B, TPD52, TPM2, TPST2, TRABD2A, TRDC, TRG-AS1, TRGC1, TRGC2, TSHZ2, TSPAN3, TULP4, UGCG, VCAN, XCL1, XCL2, ZAP70, and ZEB2.

In some embodiments, the molecular immune signature, the set of genes (or subset thereof), are used in methods to, among other things: (i) identify populations of immune cells, and/or (ii) identify diseases or conditions associated with immune cells or an immune response, and/or (iii) select or optimize treatments associated with diseases or conditions associated with immune cells or an immune response. According to some embodiments, the molecular immune signature, the set of genes (or subset thereof) is identified in accordance with the studies described below in the working examples, as well as the corresponding figures and tables disclosed therein. In certain embodiments, the full set of 220 genes are used in the methods described herein.

In other embodiments, a subset of the 220 genes is used in the methods described herein. In some embodiments, the subset of genes is about 10 or more of the genes above, about 25 or more of the genes above, about 50 or more of the genes above, about 100 or more of the genes above, about 150 or more of the genes above, or about 200 or more of the genes above. In other embodiments, the molecular immune signature includes between 1 and 25 of the genes above, between 25 and 50 of the genes above, between 50 and 100 of the genes above, between 100 and 150 of the genes above, between 150 and 200 of the genes above, or between 200 and 220 of the genes above.

For example, the full set of 220 genes may not be needed to target certain populations of cells according to some embodiments. For example, the full set of 220 genes may be further reduced by: (1) targeting limited cell subsets (e.g., T cells), or (2) using a panel-based scRNA-seq approach, where there could be increased gene detection efficiency.

In some embodiments, the relatively small set or subset of genes (e.g., a set of 220 or fewer genes) allow for methods including, but not limited to, methods that can be used to label immune cell types, visualize cell clusters for big datasets, and/or identify immune perturbed markers. Furthermore, in some embodiments, the identification of a minimal list of 220 genes required for cell typing, will allow the use of targeted panel single cell technologies that only identify a limited subset of genes, for example, 1,000 genes (220 for cell typing and 780 genes for experimental testing of cell state). The embodiments described herein have the advantage of reducing sequencing costs and also potentially overcoming the so-called dropout rate (false negatives) that are a current limitation of single cell technologies.

### Methods of Treatment

In some embodiments, provided is a method of identifying, detecting, and/or monitoring a health condition in a subject in need thereof. The health condition can be a condition that is affected by age, environmental, occupational, and/or physical factors. Alternatively, the health condition can be a disease condition. In some embodiments, the method comprises measuring the levels of a set of genes in a biological sample obtained from the subject, wherein the set of genes comprises all or a subset of the STATIC 220 genes as described. In some embodiments, the method further comprises treating the subject for the disease condition. In some embodiments, the method further comprises measuring the levels of the set of genes in a second biological sample obtained from the subject after the treatment, so that the disease condition can be monitored and/or followed over time and throughout treatment.

In some embodiments, the subset of genes is about 10 or more of the genes above, about 25 or more of the genes above, about 50 or more of the genes above, about 100 or more of the genes above, about 150 or more of the genes above, or about 200 or more of the genes as described.

In some embodiments, the biological sample is a tissue sample obtained from the subject. In some embodiments, the biological sample is a blood sample obtained from the subject, including, for example, plasma, serum, red blood cells (RBCs), and/or peripheral blood mononuclear cells (PBMCs). In some embodiments, especially in cases where the subject has cancer, the blood sample may contain circulating tumor cells (CTCs) that allow detection, diagnosis, and/or prognosis of the cancer. CTCs are tumor cells that shed from the primary tumor and intravasate into and circulate in the blood system responsible for metastasis. CTCs contain important genetic information about the cancer, and thus detection of CTCs from blood samples can serve as an effective tool.

In some embodiments, the measurement of the gene levels in the biological sample may be carried out using single cell technology. Non-limiting exemplary single cell technologies include single-cell ribonucleic acid sequencing (scRNA-seq) and single-cell assay for transposase-accessible chromatin sequencing (scATAC-seq).

In some embodiments, the disease condition is a viral infection, for example, influenza and SARS-CoV-2 infection.

In some embodiments, the disease condition is cancer. In some embodiments, the cancer is a hematological malignancy. Non-limiting exemplary hematological malignancies include monoclonal B cell lymphocytosis, multiple myeloma, myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), blast crisis chronic myelogenous leukemia (bcCML), B cell acute lymphoid leukemia (B-ALL), T cell acute lymphoid leukemia (T-ALL), T cell lymphoma, and B cell lymphoma.

In some embodiments, the cancer is a solid tumor. Non-limiting exemplary solid tumors include lung cancer, breast cancer, liver cancer, stomach cancer, colon cancer, rectal cancer, kidney cancer, gastric cancer, gallbladder cancer, cancer of the small intestine, esophageal cancer, melanoma, bone cancer, pancreatic cancer, skin cancer, uterine cancer, ovarian cancer, testicular cancer, cancer of the thyroid gland, cancer of the adrenal gland, bladder cancer, and glioma.

In some embodiments, the disease condition is an autoimmune disease. Non-limiting exemplary autoimmune diseases include type 1 diabetes, lupus, systemic lupus erythematosus, rheumatoid arthritis, psoriasis, psoriatic arthritis, multiple sclerosis, inflammatory bowel disease, Crohn's disease, ulcerative colitis, Addison's disease, Graves' disease, Sjögren's syndrome, Hashimoto's thyroiditis, myasthenia gravis, autoimmune vasculitis, pernicious anemia, and celiac disease.

In some embodiments, the methods described herein also allow researchers to label big single cell data without existing label transfer algorithm, identify immune responsive genes for viral/disease perturbed or external changes, and/or study immune cell dynamics in individual patient. As above, the methods may be used in targeted panel-based single cell sequencing technology using the 220 genes (or subset thereof) for cell typing.

In some embodiments, the set of genes or subset thereof can be used in methods for monitoring immune health and diagnosing disease. In certain embodiments, the set of 220 genes or subset thereof can be used in methods to monitor immune health for the general population. In such methods, the set of genes or subset thereof are used as a molecular signature to provide a practical and effective way to define immune health at a molecular signature level. Such methods may also provide economical methods to longitudinally monitor the health status of individuals over time, according to certain embodiments. In other embodiments, such methods may be used to identify individuals with compromised immune systems, to assess vaccine competency, or to otherwise monitor the immune health or disease state of a subject.

In other embodiments, the set of genes or subset thereof can be used in methods for optimizing or improving medical treatment of patients. In some embodiments, the methods may be used to assess immune capacity pre and post immunosuppressive or surgical intervention. In other embodiments, the methods may be used to identify acute immune signatures associated with trauma, ischemia reperfusion injury, sepsis, multiorgan dysfunction, or other conditions. In other embodiments, the methods may be used to monitor rejection signatures post organ transplantation, identify and/or diagnose possible causes for autoimmune flares, diagnose diseases, monitor and/or predict treatment outcomes, monitor disease progression, select best therapeutic intervention(s), or otherwise suitably monitor immune responses or effects thereof in a patient.

In other embodiments, the set of genes or subset thereof can be used in methods to facilitate medical research and/or drug development. In other embodiments, the set of genes or subset thereof can be used to measure effects of and understand the mechanisms of new drugs, identify patient groups with positive efficacy, or rescue failed drugs.

In some embodiments, the set of genes or subset thereof can be utilized in broad, cutting-edge applications: immune-oncology, cancer vaccines, generic TLR agonists, or other mechanism to boost immunity.

### Methods of Cell Typing

In some embodiments, provided is a method of identifying, labeling, and/or quantifying immune cell types in a biological sample. In some embodiments, the method comprises measuring levels of a set of genes in the biological sample, wherein the set of genes comprises all or a subset of the STATIC 220 genes as described.

In some embodiments, the subset of genes is about 10 or more of the genes above, about 25 or more of the genes above, about 50 or more of the genes above, about 100 or more of the genes above, about 150 or more of the genes above, or about 200 or more of the genes as described.

In some embodiments, the biological sample is a tissue sample obtained from the subject. In some embodiments, the biological sample is a blood sample obtained from the subject, including, for example, plasma, serum, RBCs, and/or PBMCs.

In some embodiments, the measurement of the gene levels in the biological sample may be carried out using single cell technology. Non-limiting exemplary single cell technologies include single-cell ribonucleic acid sequencing (scRNA-seq) and single-cell assay for transposase-accessible chromatin sequencing (scATAC-seq).

In some embodiments, the method can be used for cell typing of immune cells based on their expression levels of the immune signature genes. As demonstrated in the working examples, the STATIC 220 genes comprise genes that are unique to individual immune cell types but consistent among individual subjects. Thus, different expression patterns of the STATIC 220 genes or subset thereof can be used to distinguish different immune cell types, including, for example, B cells, T cells, natural killer (NK) cells, monocytes, macrophages, dendritic cells (DCs), mast cells, neutrophils, eosinophils, and basophils.

In some embodiments, the set of genes or subset thereof can be used to distinguish normal immune cells and abnormal (e.g., diseased) immune cells based on their expression patterns. In some embodiments, the presence and/or quantity of abnormal immune cells in a biological sample from a subject may serve as an indication of a disease condition associated with the subject, which in turn may be useful in the diagnostic methods as described.

In some embodiments, the set of genes or subset thereof can be used to perform basic biological research. For example, the set of genes or subset thereof can be used in methods to perform targeted immune profiling. Companies may offer predefined or custom panels that include the gene set (or subset thereof) for cell typing.

The set of genes and subsets thereof have several advantageous properties when used in accordance with the embodiments described herein. In some embodiments, the set of 220 genes or subset thereof can be used in methods for identifying types of peripheral blood mononuclear cells (PBMCs). Such methods are advantageous over known methods because they use a much smaller set or subset of genes (e.g., 220 or fewer genes) than the thousands of genes used in methods used by others. For example, the closest known set of genes that can be used in a similar manner is a set of 2000-3000 highly variable genes (HVGs). See Stuart et al., 2019. [PMID: 31178118].

Further, according to some embodiments, the set of genes or subset thereof can be reproducibly measured, solving the reproducibility issue suffered by the scRNA-seq platform. As mentioned above, the set of genes or subset thereof is also significantly less expensive to measure than the thousands of genes under the current technology. The set of 220 genes or subset thereof allow for better data quality by targeting a short list of genes rather than trying to measure thousands of genes. In other words, the innovation makes the scRNA-seq platform more reproducible and less expensive with little to no compromise with respect to biological insights.

Performance of the set of genes and subsets thereof in separating PBMC types has been validated in five independent datasets. Preliminary evidence for their application in monitoring disease status has been established as described in the working examples below. Their reproducibility has been quantified from data on multiple samples. Methods to target these genes and demonstrate the cheaper and better data quality claims will also be developed.

### Testing Kits and Assays

In some embodiments, provided is a testing kit or assay comprising probes for measuring the levels of a set of genes in a biological sample, wherein the set of genes comprises all or a subset of the STATIC 220 genes as described. The testing kit or assay may be used for purposes of cell typing and identifying, detecting, and/or monitoring disease conditions in a subject as described herein.

In some embodiments, the set of genes comprises about 10 or more genes, about 25 or more genes, about 50 or more genes, about 100 or more genes, about 150 or more genes, or about 200 or more genes.

In some embodiments, the testing kit or assay may be used in a single cell assay for quantifying gene levels, including, for example, single-cell ribonucleic acid sequencing (scRNA-seq) and single-cell assay for transposase-accessible chromatin sequencing (scATAC-seq).

In some embodiments, the testing kit or assay may be used on biological samples (e.g., tissue samples or blood samples) obtained from a subject. In some embodiments, the biological sample contains plasma, serum, red blood cells (RBCs), and/or peripheral blood mononuclear cells (PBMCs).

### EXAMPLES

### Example 1: PALMO: A Comprehensive Platform for Analyzing Longitudinal Multi-Omics

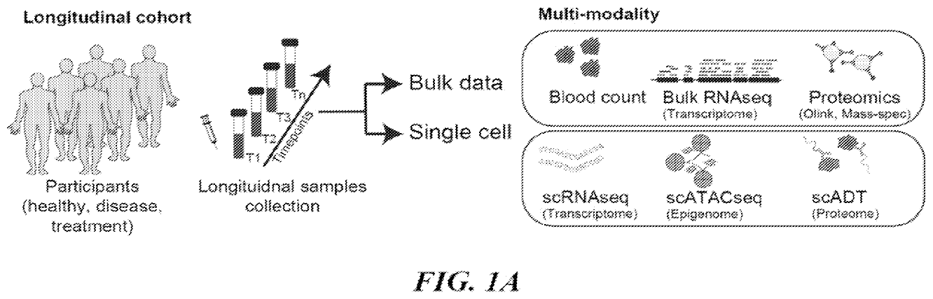

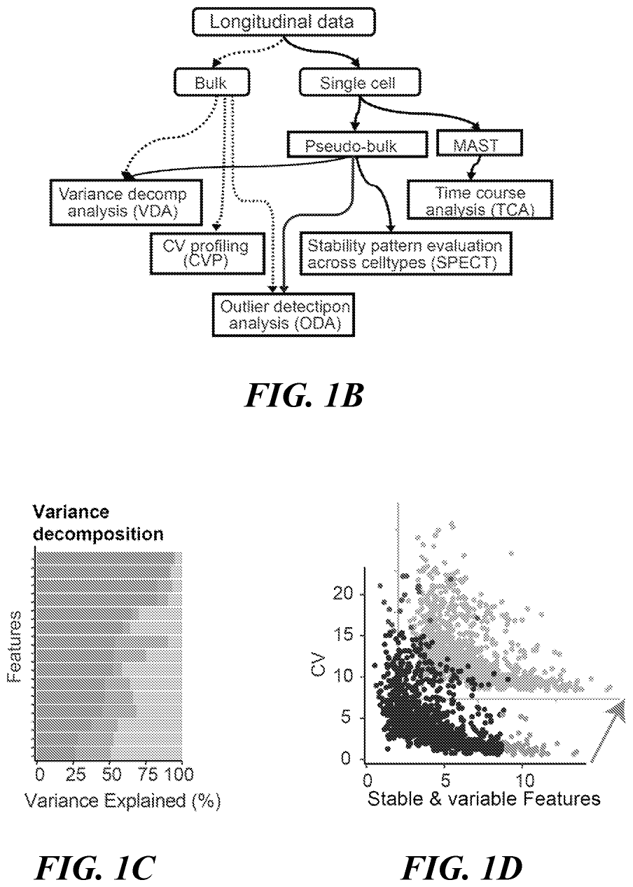

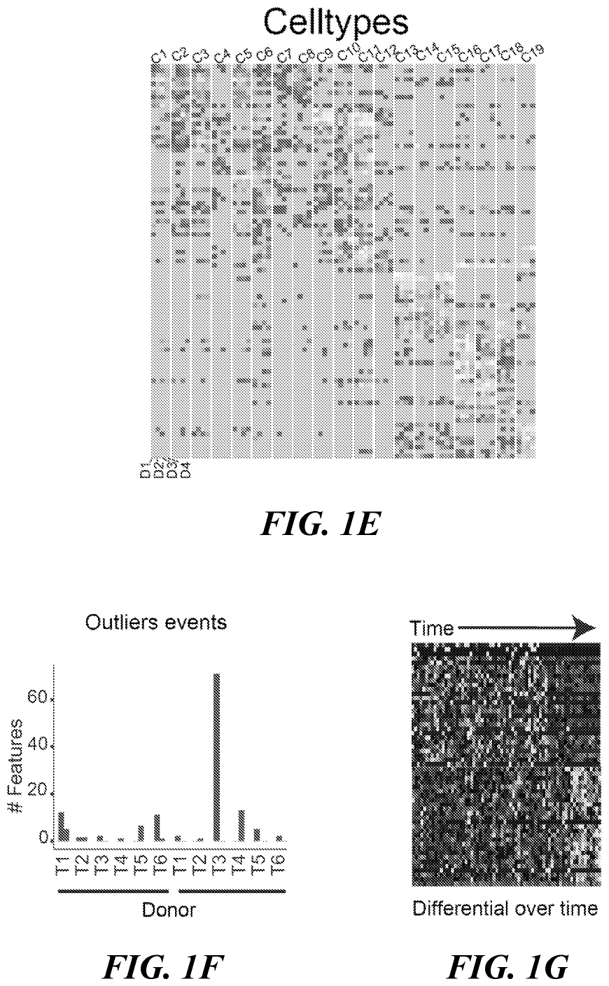

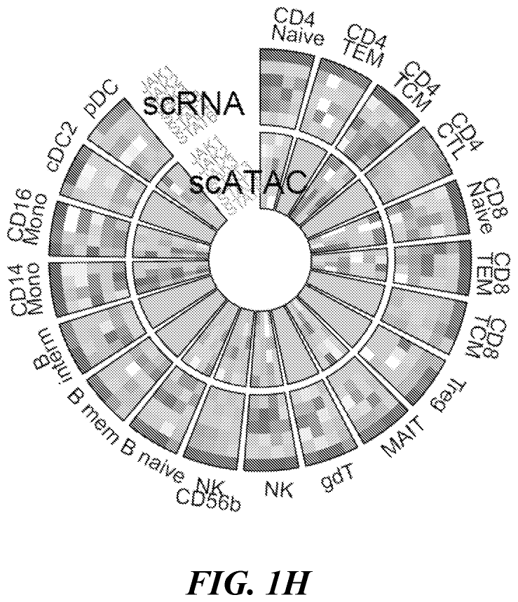

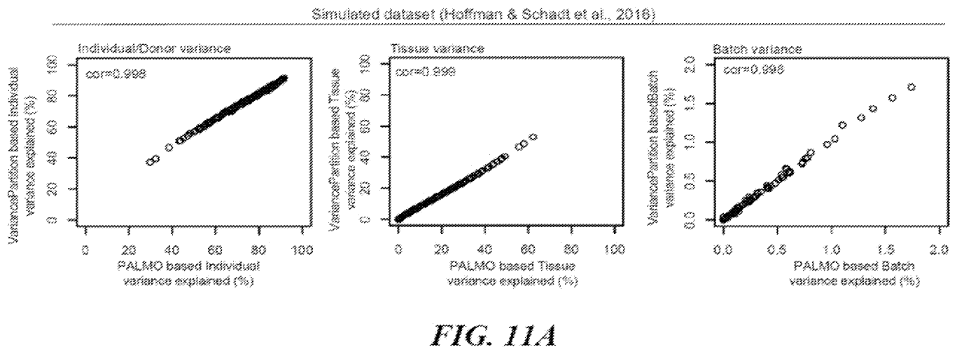

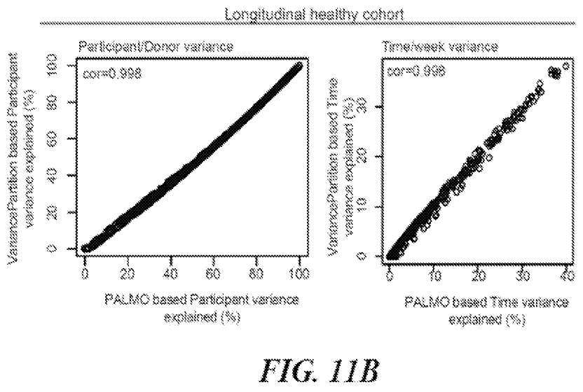

Provided herein is PALMO (https://github.com/aifimmunology/PALMO), a software package designed to analyze longitudinal bulk and single-cell omics data (FIG. 1A). Five analytical modules are implemented in PALMO (FIG. 11B): (i) variance decomposition analysis (VDA) evaluates contributions of factors of interest to the total variance of individual features (FIG. 1C); (ii) coefficient of variation (CV) profiling (CVP) assesses intra-participant variation over time in bulk data and identifies consistently stable or variable features among participants (FIG. 1D); (iii) stability pattern evaluation across cell types (SPECT) assesses longitudinal stability patterns of features in single-cell omics data and identifies stable or variable features that are unique to individual cell types but consistent among participants (FIG. 1E); (iv) outlier detection analysis (ODA) examines the possibility of abnormal events occurring during a longitudinal study (FIG. 1F); (v) time course analysis (TCA) evaluates transcriptomic changes over time based on longitudinal scRNA-seq data of the same participant and identifies genes that exhibit significant temporal changes (FIG. 1G). Together, these five modules provide unique insights on longitudinal omics data from multiple perspectives. Functions to display CVs of features of interest in circos plots were also developed (FIG. 1H).

The following methods are used in Examples 2-9 below:

Healthy donors: We enrolled six clinically healthy donors (no diagnosis of active or chronic disease) with age between 25 to 38 years with equal sex ratio. Blood samples were obtained from Bloodworks Northwest (Seattle, WA) through protocols approved by the Bloodworks Northwest institutional review board. The cohort demographics are described in the Table 2A. Viable peripheral blood mononuclear cells (PBMCs) and plasma samples were collected from each donor over 10 weeks. Complete blood count (CBC) was measured to evaluate overall health of all donors over all timepoints (n=6, t=10). Minimal biometric data were collected on these donors which were handled following the Health Insurance Portability and Accountability Act (HIPAA) guidelines.

Sample handling: A volume of 30 mL of blood was drawn into BD NaHeparin vacutainer tubes (for PBMC; BD #367874) or K2-EDTA vacutainer tubes (for plasma; BD #367863). Upon arrival at the processing lab all NaHeparin tubes for each donor were pooled into a sterile plastic receptacle to establish one common pool and stored at room temperature until processing (4 hours or less from draw). PBMC were isolated by Ficoll density gradient separation and cryopreserved by a team of operators, as previously described. Thawed PBMC of four donors over six timepoints (n=4, t=6) were assayed by flow cytometry, scRNA-seq and scATAC-seq in two batches (donors PTID5 and PTID6, donors PTID2 and PTID4) by a team of operators. Plasma of all donors over all timepoints (n=6, t=10) was isolated and cryopreserved by a team of operators, as previously described.

Flow cytometry: Flow cytometry was performed as previously described. In brief, cryopreserved PBMC were thawed, washed, and counted. 1-2×10⁶ cells were incubated with Human TruStain FcX (BioLegend #422302) and Fixable Viability Stain 510 (BD #564406) prior to staining with a 25-color cell surface panel (Key Resources Table) on ice for 25 minutes. Cells were washed and fixed with 4% paraformaldehyde (Electron Microscopy Sciences #15713) prior to acquisition on a BD Symphony cytometer. Raw data were compensated and curated to remove unrepresentative events due to instrument fluidics variability (time gating), doublets (by FSC-H and FSC-W), and cells exhibiting membrane permeability (live/dead gating) prior to quantification using BD FlowJo software.

Proteomics: Plasma samples were submitted to Olink (Uppsala, Sweden) for assay using the Olink Proximity Extension assay, run on the Fluidigm Biomark system. Patient samples were distributed evenly across two plates, and all time points per patient were run on the same plate, with randomized well locations. Samples were assayed using the Olink Discovery Assay which encompasses a total of 1536 proteins across 13 panels (Cardiometabolic [V.3603], Cardiovascular II [V.5006], Cardiovascular III [V.6113], Cell Regulation [V.3701], Development [V.3512], Immune Response [V.3202], Inflammation [V.3021], Metabolism [V.3402], Neuro Exploratory [V.3901], Neurology [V.8012], Oncology 11 [V.7004], Oncology III [V.4001], Organ Damage [V.3311]). Quality assessment, limit of detection, and normalization were performed by Olink using the plate bridging control, two positive controls, and three background controls.

### Single-Cell RNA-Seq:

Sample preparation, hashing, and pooling: Single-cell RNA-seq libraries were generated using the 10× Genomics Chromium 3′ Single Cell Gene Expression assay (#1000121) and Chromium Controller Instrument according to the manufacturer's published protocol with modifications for cell hashing. To block off-target antibody binding, Blocking Solution (5 μL of Human TruStain FcX (BioLegend #422302), and 13.7 μL of a 10% Bovine Serum Albumin (BSA)) was added to 500,000 cells suspended in 50 μL Dulbecco's Phosphate Buffered Saline (DPBS; Corning Life Sciences #21-031-CM) and incubated for 10 minutes on ice. To stain samples, 0.5 μg (1 μL) of a TotalSeq™-A anti-human Hashtag Antibody was suspended in 31.3 μL DPBS/2% BSA, then added to each sample. For each batch of samples, 100,000 cells from 12 hashed samples with a distinct Hashtag Antibody were pooled into the hashed pool. Roughly 20,000 cells from a Leukopak healthy control were also labeled with a distinct TotalSeq™-A Hashtag Antibody and were spiked into each pool to serve as a batch control.

Droplet encapsulation and reverse transcription: From each pool, 64,000 cells were loaded into each well of a Chromium Single Cell Chip G (10× Genomics #1000073) (8 wells per chip), targeting a recovery of 20,000 singlets from each well. Gel Beads-in-emulsion (GEMs) were then generated using the 10× Chromium Controller. The resulting GEM generation products were then transferred to semi-skirted 96-well plates and reverse transcribed on a C1000 Touch Thermal Cycler (Bio-Rad) programmed at 53° C. for 45 minutes, 85° C. for 5 minutes, and a hold at 4° C. Following reverse transcription, GEMs were broken, and the pooled single-stranded cDNA and Hashtag Oligo fractions were recovered using Silane magnetic beads (Dynabeads MyOne SILANE #37002D).

Library generation and separation: Barcoded, full-length cDNA including the Hashtag Oligos (HTOs) from the TotalSeq™-A Hashtag Antibodies were then amplified with a C1000 Touch Thermal Cycler programmed at 98° C. for 3 minutes, 11 cycles of (98° C. for 15 seconds, 63° C. for 20 seconds, 72° C. for 1 minute), 72° C. for 1 minute, and a hold at 4° C. Amplified cDNA was purified and separated from amplified HTOs using a 0.6× size selection via SPRIselect magnetic bead (Beckman Coulter #22667) and a 1:10 dilution of the resulting cDNA was run on a Fragment Analyzer (Agilent Technologies #5067-4626) to assess cDNA quality and yield. HTO libraries were purified further with SPRIselect magnetic bead (Beckman Coulter #22667) and amplified and indexed with a custom HTO i7 index on a C1000 Touch Thermal Cycler programmed at 95° C. for 3 minutes, 10 cycles of (95° C. for 20 seconds, 64° C. for 30 seconds, 72° C. for 20 seconds), 72° C. for 1 minute, and a hold at 4° C. The resulting HTO libraries were purified with SPRIselect magnetic bead (Beckman Coulter #22667) post-amplification and a 1:10 dilution of the resulting HTO libraries were run on a Fragment Analyzer (Agilent Technologies #5067-4626) to assess HTO quality and yield. A quarter of the cDNA sample (10 ul) was used as input for library preparation. Amplified cDNA was fragmented, end-repaired, and A-tailed is a single incubation protocol on a C1000 Touch Thermal Cycler programmed at 4° C. start, 32° C. for minutes, 65° C. for 30 minutes, and a 4° C. hold. Fragmented and A-tailed cDNA was purified by performing a dual-sided size selection using SPRIselect magnetic beads (Beckman Coulter #22667). A partial TruSeq Read 2 primer sequence was ligated to the fragmented and A-tailed end of cDNA molecules via an incubation of 20° C. for 15 minutes on a C1000 Touch Thermal Cycler. The ligation reaction was then cleaned using SPRIselect magnetic beads (Beckman Coulter #22667). PCR was then performed to amplify the library and add the P5 and indexed P7 ends (10× Genomics #1000084) on a C1000 Touch Thermal Cycler programmed at 98° C. for 45 seconds, 13 cycles of (98° C. for 20 seconds, 54° C. for 30 seconds, 72° C. for 20 seconds), 72° C. for 1 minute, and a hold at 4° C. PCR products were purified by performing a dual-sided size selection using SPRIselect magnetic beads (Beckman Coulter #22667) to produce final, sequencing-ready libraries.

Quantification and sequencing: Final libraries were quantified using Picogreen and their quality was assessed via capillary electrophoresis using the Agilent Fragment Analyzer HS DNA fragment kit and/or Agilent Bioanalyzer High Sensitivity chips. Libraries were sequenced on the Illumina NovaSeq platform using S4 flow cells. Read lengths were 28 bp read1, 8 bp i7 index read, 91 bp read2.

scRNA-seq data pre-processing: scRNA-seq data of four donors were generated in two batches, each containing data of two donors. Each batch of data was pre-processed separately as previously described. Briefly, binary base call (BCL) files were demultiplexed using the mkfastq function in the 10× Cell Ranger software (version 3.1.0), producing fastq files. Fastq files were then checked for quality (FastQC version 0.11.3) and run through the 10× Cell Ranger alignment function (cell ranger count) against the human reference annotation (Ensembi GRCh38). Mapping was performed using default parameters. Upon completion, Cell Ranger produced an output directory per file that contains the following: bam file (binary alignment file), HDF5 file (Hierarchical Data Format) with all reads, HDF file containing just the filtered reads, summary report (html and csv), and cloupe.cloupe (a file for the 10× Loupe visual browser).

scRNA-sea data analysis: As previously described, individual HDF5 files (filtered) were loaded into the R statistical programming language (version 3.6.0) using Bioconductor (version 3.1.0) and the Seurat package (version 3.1.5). For simplicity, sample names were captured as a list in R and iteratively processed within a loop (refer to https://satijalab.org/seurat/for more information). Within the loop, samples were normalized with the NormalizeData function followed by the FindVariableFeatures function with parameters: vst selection method and 2000 features. Label transfer was performed using previously published procedures and with the Seurat reference dataset. Labeling included the FindTransferAnchors and TransferData functions performed in the Seurat package.

We merged the two batches of data using the Seurat merge function. We calculated read depth, mitochondrial percentage, and number of UMIs per sample. Cells were filtered with nFeature_RNA>200 and percent.mt<10. The merged data structure was normalized (using NormalizeData and FindVariableFeatures functions) and then saved as an RDS for further analysis. The top 3000 variable genes were used for principal component analysis (PCA) and UMAP based dimension reduction maps using 30 principal components (PCs). We checked for possible batch effects using the bridging controls but did not observe any obvious batch effects.

Cell labels obtained from the original batches were kept. Doublets were removed from further analysis. In total we retrieved high quality data of 472,464 cells from 4 donors and labeled them to 31 cell types from Seurat V2. The cell type frequencies in each sample were calculated and compared with flow-based cell frequencies. Nineteen cell types (CD4_Naive, CD4_TEM, CD4_TCM, CD4_CTL, CD8_Naive, CD8_TEM, CD8_TCM, Treg, MAIT, gdT, NK, NK_CD56bright, B_naive, B_memory, B_intermediate, CD14_Mono, CD16_Mono, cDC2, pDC) were selected for further analysis after filtering out cell types with a low frequency (<0.5%).

### Single-cell ATAC-seq:

Sample preparation: Permeabilized-cell scATAC-seq was performed as described previously (Swanson et al. 2021). A 5% w/v digitonin stock was prepared by diluting powdered digitonin (MP Biomedicals, 0215948082) in DMSO (Fisher Scientific, D12345), which was stored in 20 μL aliquots at −20° C. until use. To permeabilize, 1×106 cells were added to a 1.5 mL low binding tube (Eppendorf, 022431021) and centrifuged (400×g for 5 min at 4° C.) using a swinging bucket rotor (Beckman Coulter Avanti J-15RIVD with JS4.750 swinging bucket, B99516). Cells were resuspended in 100 μL cold isotonic Permeabilization Buffer (20 mM Tris-HCl pH 7.4, 150 mM NaCl, 3 mM MgCl2, 0.01% digitonin) by pipette-mixing 10 times, then incubated on ice for 5 min, after which they were diluted with 1 mL of isotonic Wash Buffer (20 mM Tris-HCl pH 7.4, 150 mM NaCl, 3 mM MgCl2) by pipette-mixing five times. Cells were centrifuged (400×g for 5 min at 4° C.) using a swinging bucket rotor, and the supernatant was slowly removed using a vacuum aspirator pipette. Cells were resuspended in a chilled TD1 buffer (Illumina, 15027866) by pipette-mixing to a target concentration of 2,300-10,000 cells per μL. Cells were filtered through 35 μm Falcon Cell Strainers (Corning, 352235) before counting on a Cellometer Spectrum Cell Counter (Nexcelom) using ViaStain acridine orange/propidium iodide solution (Nexcelom, C52-0106-5).

Tagmentation and fragment capture: scATAC-seq libraries were prepared according to the Chromium Single Cell ATAC v1.1 Reagent Kits User Guide (CG000209 Rev B) with several modifications. 19,000 cells were loaded into each tagmentation reaction. Permeabilized cells were brought up to a volume of 12 μl in TD1 buffer (Illumina, 15027866) and mixed with 3 μl of Illumina TDE1 Tn5 transposase (Illumina, 15027916). Transposition was performed by incubating the prepared reactions on a C1000 Touch Thermal Cycler with 96-Deep Well Reaction Module (Bio-Rad, 1851197) at 37° C. for 60 minutes, followed by a brief hold at 4° C. A Chromium NextGEM Chip H (10× Genomics, 2000180) was placed in a Chromium Next GEM Secondary Holder (10× Genomics, 3000332) and 50% Glycerol (Teknova, G1798) was dispensed into all unused wells. A master mix composed of Barcoding Reagent B (10× Genomics, 2000194), Reducing Agent B (10× Genomics, 2000087), and Barcoding Enzyme (10× Genomics, 2000125) was then added to each sample well, pipette-mixed, and loaded into row 1 of the chip. Chromium Single Cell ATAC Gel Beads v1.1 (10× Genomics, 2000210) were vortexed for 30 seconds and loaded into row 2 of the chip, along with Partitioning Oil (10× Genomics, 2000190) in row 3. A 10× Gasket (10× Genomics, 370017) was placed over the chip and attached to the Secondary Holder. The chip was loaded into a Chromium Single Cell Controller instrument (10× Genomics, 120270) for GEM generation. At the completion of the run, GEMs were collected, and linear amplification was performed on a C1000 Touch Thermal Cycler with 96-Deep Well Reaction Module: 72° C. for 5 min, 98° C. for 30 sec, 12 cycles of: 98° C. for 10 sec, 59° C. for 30 sec and 72° C. for 1 min.

Sequencing library preparation: GEMs were separated into a biphasic mixture through addition of Recovery Agent (10× Genomics, 220016), the aqueous phase was retained and removed of barcoding reagents using Dynabead MyOne SILANE (10× Genomics, 2000048) and SPRIselect reagent (Beckman Coulter, B23318) bead clean-ups. Sequencing libraries were constructed by amplifying the barcoded ATAC fragments in a sample indexing PCR consisting of SI-PCR Primer B (10× Genomics, 2000128), Amp Mix (10× Genomics, 2000047) and Chromium i7 Sample Index Plate N, Set A (10× Genomics, 3000262) as described in the 10× scATAC User Guide. Amplification was performed in a C1000 Touch Thermal Cycler with 96-Deep Well Reaction Module: 98° C. for 45 sec, for 11 cycles of: 98° C. for 20 sec, 67° C. for 30 sec, 72° C. for 20 sec, with a final extension of 72° C. for 1 min. Final libraries were prepared using a dual-sided SPRIselect size selection cleanup. SPRIselect beads were mixed with completed PCR reactions at a ratio of 0.4× bead:sample and incubated at room temperature to bind large DNA fragments. Reactions were incubated on a magnet, the supernatant was transferred and mixed with additional SPRIselect reagent to a final ratio of 1.2× bead:sample (ratio includes first SPRI addition) and incubated at room temperature to bind ATAC fragments. Reactions were incubated on a magnet, the supernatant containing unbound PCR primers and reagents was discarded, and DNA bound SPRI beads were washed twice with 80% v/v ethanol. SPRI beads were resuspended in Buffer EB (Qiagen, 1014609), incubated on a magnet, and the supernatant was transferred resulting in final, sequencing-ready libraries.

Quantification and sequencing: Final libraries were quantified using a Quant-iT PicoGreen dsDNA Assay Kit (Thermo Fisher Scientific, P7589) on a SpectraMax iD3 (Molecular Devices). Library quality and average fragment size was assessed using a Bioanalyzer (Agilent, G2939A) High Sensitivity DNA chip (Agilent, 5067-4626). Libraries were sequenced on the Illumina NovaSeq platform with the following read lengths: 51 nt read 1, 8 nt i7 index, 16 nt i5 index, 51 nt read 2.

scATAC data pre-processing: scATAC-seq data were available for donor PTID2 and PTID4 at week 2-7 (6 timepoints) and for PTID5 and PTID6 at week 2, 4, and 7. scATAC-seq libraries were processed as described previously (Swanson et al., 2021a). In brief, cellranger-atac mkfastq (10× Genomics v1.1.0) was used to demultiplex BCL files to FASTQ. FASTQ files were aligned to the human genome (10× Genomics refdata-cellranger-atac-GRCh38-1.1.0) using cellranger-atac count (10× Genomics v1.1.0) with default settings. scATAC fragments were submitted to the ArchR package to create the ArchR object. Per-cell quality control (QC) was performed using methods as mentioned in ArchR. The QC analysis showed FRiP score (the fraction of reads that fall into a peak) >0.25. The TSS enrichment and log 10(nFrags) data showed comparable range across all samples. Doublets were removed using filterDoublets( ) function. In total we observed 294,623 peaks in 135,566 cells.

scATAC-sea data analysis: Using plotEmbedding function in ArchR, embedded IterativeLSI was used to perform UMAP based dimension reduction. Unconstrained integration was used to align scATAC-seq gene score matrix in ArchR object with the corresponding scRNA-seq gene expression matrix, from which cells were labeled to 28 cell types along with labeling scores to measure the quality of the cell-label transfer. We filtered out low quality cells (labeling score <0.5), removed cell types having less than 50 remaining cells, and kept 14 (B_intermediate, B_naive, CD14_Mono, CD16_Mono, CD4_Naive, CD4_TCM, CD8_Naive, CD8_TEM, cDC2, gdT, MAIT, NK, NK_CD56bright, and pDC) out of the 28 cell types for downstream analysis. The gene score matrix was retrieved using the getGroupSE( ) function in ArchR and used for downstream analysis by PALMO.

### PALMO:

Overview: The current version of PALMO contains five analytical modules to analyze longitudinal omics data from multiple perspectives. It treats longitudinal omics data as continuous variables. PALMO has been published as an R package in CRAN with a detailed reference manual and vignettes to demonstrate its usage (https://cran.r-project.org/web/packages/PALMO/index.html). It can be easily installed and executed in R or RStudio.

PALMO S4 object: PALMO is a R based package that uses the setClass function to create an S4 object oriented system. The S4 object consists of a list of data structures with different types of elements such as strings, numbers, vectors, embedded lists, etc. It stores input expression data, input metadata, and output results into separate data structures for easy retrieval and interpretation. More details can be found in Section 3.9 of PALMO vignettes (https://raw.githubusercontent.com/aifimmunology/PALMO/mainVignette-PALMO.pdf).

Function createPALMOobject( ) takes two inputs (anndata and data) to create an PALMO S4 object: anndata is a data frame containing sample annotations. For longitudinal bulk data, data is a data frame with features (such as genes or proteins) as rows, samples as columns, and expression values as elements. For longitudinal single-cell omics data, data is a Seurat object. For single-cell omics data without a Seurat object, function createPALMOfromsinglecellmatrix( ) first creates a Seurat object from an expression matrix or data frame and then creates a PALMO S4 object. Function annotateMetadata( ) assigns columns in the original sample annotation data to designated variables (sample_column, donor_column, and time_column) of the PALMO object for longitudinal analysis. Function mergePALMOdata( ) cleans up the PLAMO object by filtering out data missing essential information on sample_column, donor_column, or time_column. Function checkReplicates( ) first checks whether there are replicated samples at the same time points and of the same participants and, if yes, takes the median values among replicated samples. Function avgExpCalc( ) carries out pseudo-bulking on single-cell omics data. Function naFilter( ) filters out data whose fraction of NAs is above na_cutoff (default: 0.4).

Variance decomposition analysis (VDA): For variance decomposition, we want to evaluate contributions from factors of interest ᵢ} to the variance of analyte Y with or without the influence of fixed effects ⱼ}. Some ᵢ} and ⱼ} may be the same variables. We treat Fᵢ as random effects in a linear mixed model, that is, with fixed effects,

$$Y ∼ {\text{?}}_{1} + {\text{?}}_{2} + … + {\text{?}}_{n} + ( 1 \vert {\text{?}}_{1} ) {( 1 {\vert}_{2} )}_{n} . \qquad ( 1 )$$

$$\text{?} \text{indicates text missing or illegible when filed}$$

Or, without fixed effects,

$$Y ∼ ( 1 \vert {\text{?}}_{1} ) {( 1 {\vert}_{2} )}_{n} . \qquad ( 2 )$$

$$\text{?} \text{indicates text missing or illegible when filed}$$

Using Ime4, one can obtain the corresponding variance i2, including the residual variance R2. Then the total variance of y is given by

$$\begin{matrix}2 & 2 & 2 & 2 & 2 \\ total & 1 & 2 & n & R\end{matrix} . \qquad ( 3 )$$

The proportion of variance explained by factor i is then i2total2. Similar approach was used in variancePartition where the percentage of variance explained was interpreted as the intra-class correlation (ICC). VDA can be performed with the function ImeVariance( ).

Coefficient of variation (CV) profiling (CVP): CVP is designed for bulk longitudinal data and contains two functions: (1) Function cvCalcBulkProfile( ) calculates CV of all features and generates the corresponding CV profile. (2) Function cvCalcBulk( ) identifies consistently stable and variable features, which has two important parameters: Parameter cvThreshold (default: 5%) specifies the CV cutoff for distinguishing stable (CV<cvThreshold) or variable (CV>cvThreshold) features. Parameter donorThreshold (default: the total number of donors) defines the minimum number of donors on which a feature needs to be stable or variable to be considered as consistently stable or variable. One may choose cvThreshold as the mode of the corresponding CV distribution.

Stability pattern evaluation across cell types (SPECT): SPECT is the CVP counterpart for single-cell data and contains the following functions: (1) Function cvCalcSCProfile( ) calculates the CVs of all features in individual cell types and of individual donors and generates the corresponding CV profile. (2) Function cvSCsampleprofile( ) calculates the CVs of all features of individual donors regardless of difference in cell types and generates the corresponding CV profile. (3) Function cvCalcSC( ) determines whether individual features are stable (CV<cvThreshold) or variable (CV>cvThreshold) in individual cell types and of individual donors. One may choose cvThreshold as the mode of the corresponding CV distribution or a convenient value based on the CVs of housekeeping genes. (4) Function VarFeatures( ) first counts how many times individual features are variable in cell type-donor combinations and then classifies variable features as follows: Features whose counts are above parameter groupThreshold are classified as super variable (SUV). Features whose counts are below groupThreshold but which are consistently variable across all donors in at least one cell type are classified as variable across lime in cell-types (VATIC). The default groupThreshold value is set to N_{donor}*N_{cell type}/2, where N_{donor} is the number of donors and N_{cell type} is the number of cell types. (5) Function StableFeatures( ) is similar to VarFeatures( ) but classifies stable features as super stable (SUS) or stable across lime in cell-types (STATIC). (6) Function dimUMAPPlot( ) generates a UMAP plot using a set of selected genes as input.

Outlier detection analysis (ODA): ODA applies both graphic and statistical methods to examine the temporal behavior of longitudinal data. Function sample_correlation( ) calculates intra- and inter-donor correlations (across analytes) and displays the results in a heatmap. Timepoints showing obvious weaker correlations with other timepoints are potential outliers. To detect abnormal timepoints, function outlierDetect( ) first calculates the mean and the standard deviation (SD) of each analyte from samples of the same donor across all timepoints, calculates z=value-mean/SD for the analyte at individual timepoints, and then counts at individual timepoints how many analytes are outliers with |z|>z₀, where z₀ is a user selected cutoff value. Assuming z follows a normal distribution, it is straightforward to calculate the expected rate r of analytes having |z|>z₀ (two-sided) or having z>z₀ or z<−z₀ (one-sided). Afterwards function outlierDetectP( ) uses binomial tests to evaluate the p-values for the counts of outliers at individual timepoints and applies Benjamini and Hochberg procedure to adjust the p-values since multiple timepoints are tested. A donor-specific abnormal timepoint is identified if the corresponding adjusted p value is less than 0.05. In this study we chose z₀=2.5 and thus r=1.24% for |z|>2.5 or r=0.62% for z>2.5 or z<−2.5. While the z method described here can handle data with only three timepoints, Dixon's test may be a better alternative for such a small dataset.

Time course analysis (TCA): Function sclongitudinalDEG( ) uses the hurdle model implemented in the MAST package (https://github.com/RGLab/MAST/) to study temporal changes in longitudinal scRNA-seq data. The data is first split into subsets of individual cell types and individual participants and then analyzed independently. If the data has at least three timepoints, the function models normalized expression of each gene as a linear function of time and evaluates the slope of time and the corresponding p value (likelihood ratio test). If the data has only two timepoints, the function performs DEG analysis between the two timepoints as implemented in MAST and obtains fold change and the corresponding p value. Potential confounding factors (such as experimental batch, sex, age, etc.) can be specified by parameter adjfac which are adjusted in the analysis. Genes that are expressed in less than a certain fraction of cells (specified by parameter mincellsexpressed, default 0.1) are filtered out from the analysis. Obtained p values are adjusted for multiple comparisons using the Benjamini and Hochberg procedure. Adjusted p value<0.05 were considered significant in this study.

Circos plots for displaying stability patterns: PALMO has two functions to show the stability patterns of single-cell omics data. Function genecircosPlot( ) displays the CV values of features of interest in individual cell types and across individual donors based on a single data modality. Function multimodalView( ) displays the CV values of features of interest in individual cell types and across individual donors based on two independent data modalities.

Random correlation between gene expression and gene score: To generate the distribution of random correlation between gene expression in scRNA-seq data and gene score in scATAC-seq data, we randomly shuffled the order of reliable genes, calculated the correlations between expression of pre-shuffle genes and gene score of post-shuffle genes at the same positions, and repeated the process 1000 times. The obtained distribution of correlations provided a good estimate on the correlation between random, unrelated gene pairs, which had a 95% upper confidence bound at R₀=0.399. Any correlations below R₀ were no better than that between random, unrelated gene pairs and thus not statistically meaningful.

Published sinale cell datasets: We retrieved scRNA-seq data from published PBMC datasets CNP0001102, GSE149689, and GSE164378. Datasets CNP0001102 and GSE164378 were from longitudinal studies. Single-cell data objects were created in Seurat V4 and cells were labeled as in the original studies. Zhu et al., 2020 (CNP0001102) dataset consists of three healthy controls (normal), two participants infected with influenza (Flu) and five participants infected with SARS-CoV-2 (COVID-19). Lee et al., 2020 (GSE149689) dataset consists of four normal, five Flu, and eleven COVID-19 participants. The Hao et al., 2021 (GSE164378) dataset consists of eight participants with PBMC samples collected at three timepoints.

Mouse brain scRNA-seq data was obtained from Ximerakis et al (2019) published dataset (GSE129788). The dataset contains single cell RNA data from brain tissues of eight young (2-3 months) and eight old (21-23 months) mice. The dataset consists of a total 37,069 cells labeled to 25 cell types.

TCRß repertoire dataset: We downloaded the TCRβ sequencing data of 4 systemic sclerosis patients from GSE156980. First, we merged the TCR repertoire data from the 4 patients with 3 timepoints into a single file. Second, we calculated the frequency of each unique CDR3 peptide in each patient sample as the ratio between the observed reads of the peptide to the total peptide reads in the sample. Third, we termed unique CDR3 peptides as clonotypes and labeled them from 1 to the total number of clonotypes. In total, we collected 288,597 (out of 355,024) unique clonotypes from CD4+ T cells and 11,739 (out of 14,883) from CD8+ T cells, respectively. The frequency data matrix from CD4+ or CD8+ T cells was then submitted to PALMO as input data frame.

Differential expression gene (DEG) analysis on scRNA-sea data: DEG analysis on datasets (CNP0001102 and GSE149689) was performed using the FindMarkers function from the Seurat package (version 3.1.5). The groups were specified using “ident.1” and “ident.2” in the function. The Benjamini and Hochberg (BH) procedure as implemented in the Seurat package was applied to adjust p values, controlling the false discovery rate (FDR) in multiple testing. DEGs were identified if the corresponding average log 2-Fold change was greater than 0.1 and the corresponding adjusted p value was less than 0.05.

Seurat differential analysis on longitudinal scRNA-sea data of a COVID19 patient: Seurat based differential analysis was performed on the longitudinal scRNA-seq data of activated CD4+ T cells of patient COV-5 in Zhu et al., 2020 (CNP0001102), using the function FindMarkers( ) with parameters test.use=“MAST” and log fc.threshold=0. The groups were defined by parameters ident.1 and ident.2. For example, to capture differential genes between day 1 (D1) versus day 7 (07) and day 13 (013), we selected ident.1-D1 and ident.2=(D7 and D13). Similar approach was carried out for comparing D13 versus D1 and D7 (ident.1=(D1 and D7) and ident.2=D13). The significant genes were identified by adjusted pvalue <0.05.

Pathway enrichment analysis: Fast Gene Set Enrichment Analysis (fgsea) was performed to identify enriched pathways among targeted genes. A custom collection of gene sets that included the GO v7.2, KEGG v7.2 and Hallmark v7.2 from the Molecular Signatures Database (MSigDB, v7.2) were used as the pathway database. Genes were pre-ranked by the decreasing order of their correlation or changes or coefficients. The running sum statistics and Normalized Enrichment Scores (NES) were calculated for each comparison. The pathway enrichment p values were adjusted using the Benjamini and Hochberg procedure and pathways with adjusted p values <0.05 were considered significantly enriched. Over representation analysis was performed using the Fisher test. For a single sample GSEA (ssGSEA), we used the GSVA v1.40 R package.

Data analysis and visualization: Data analysis was performed in R, a statistical computing language (https://www.R-project.org/). Basic data visualization was performed using ggplot2 v3.3, ggpubr 0.4, and circular plots by circlize v0.4. The UMAP visualization was performed using Seurat v4. Statistical tests were performed as mentioned in each section. Multi-test correction was applied to the p values to control the FDR using the Benjamini and Hochberg procedure and adjusted p<0.05 were considered significant.

### Example 2: A Complex Longitudinal Multi-Omics Dataset to Demonstrate PALMO Performance

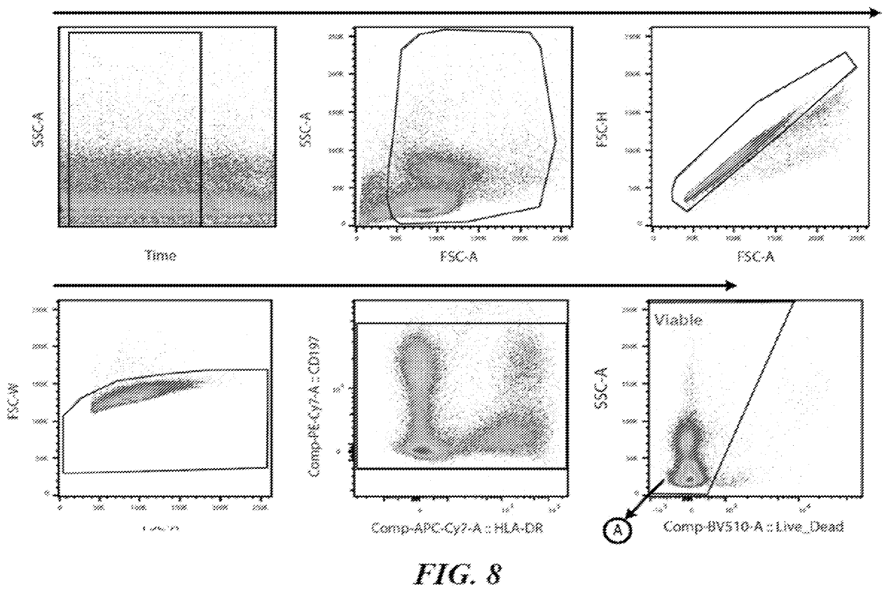

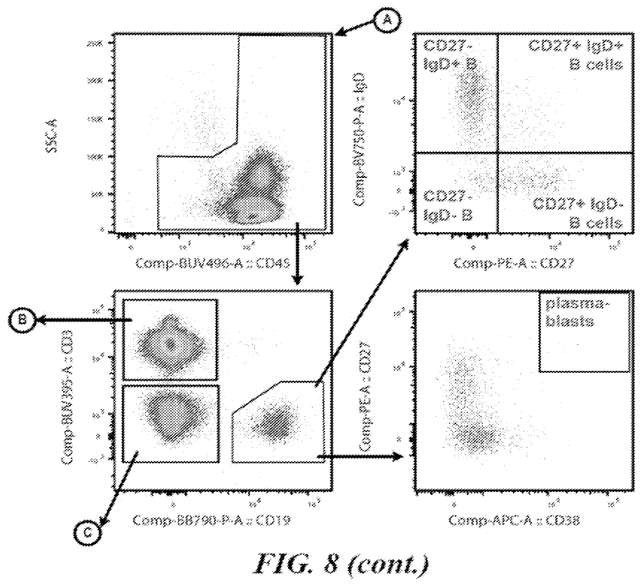

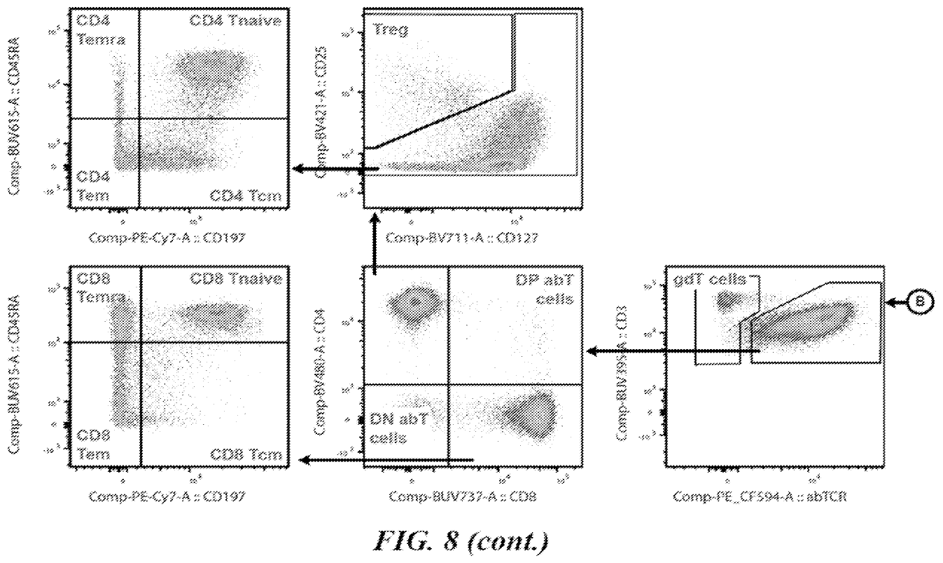

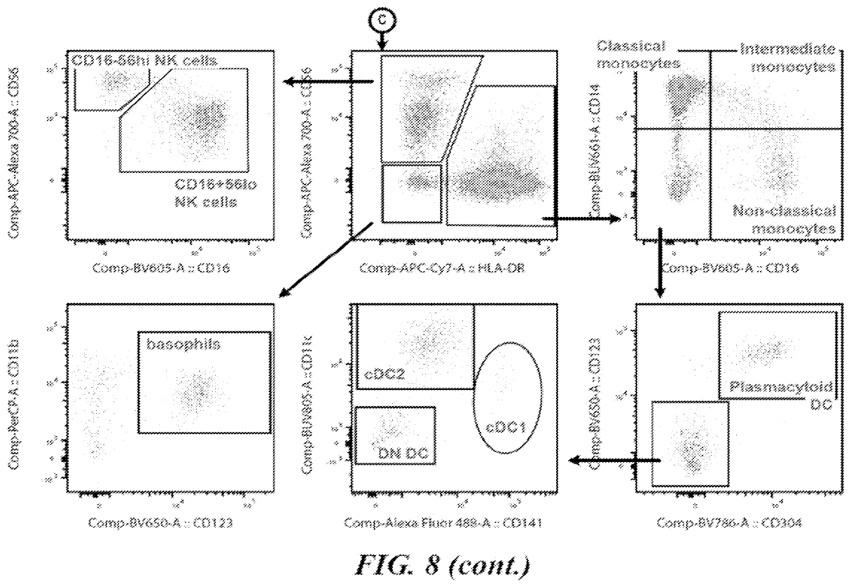

To demonstrate PALMO performance, we collected sixty blood samples (plasma and peripheral blood mononuclear cells (PBMCs)) from six healthy, non-smoking Caucasian donors (three females and three males) between 25 to 38 years old over a 10-week period (Tables 1A, 2A). Complete blood count (CBC) was collected on all these samples (Table 2B). The abundance of 1,536 plasma proteins were measured on these samples as well, but only 1,042 (68%) proteins had reliable quantification results (Table 3A). High-dimensional flow cytometry and droplet-based scRNA-seq assays were performed on a subset of 24 PBMC samples from four donors over Week 2 to 7. A total of 27 cell types were identified from flow cytometry data (FIG. 8, Table 2C). Droplet-based scATAC-seq assay was also performed on 18 out of the 24 PBMC samples. This multi-omics dataset of five data modalities on the same samples can be a valuable resource for immune health study.

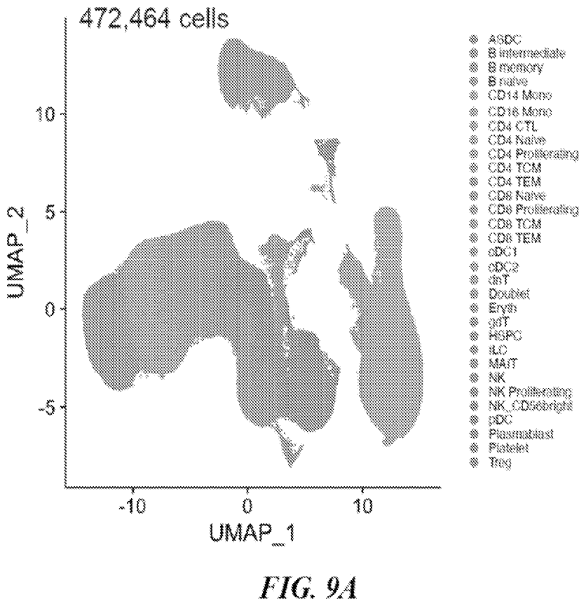

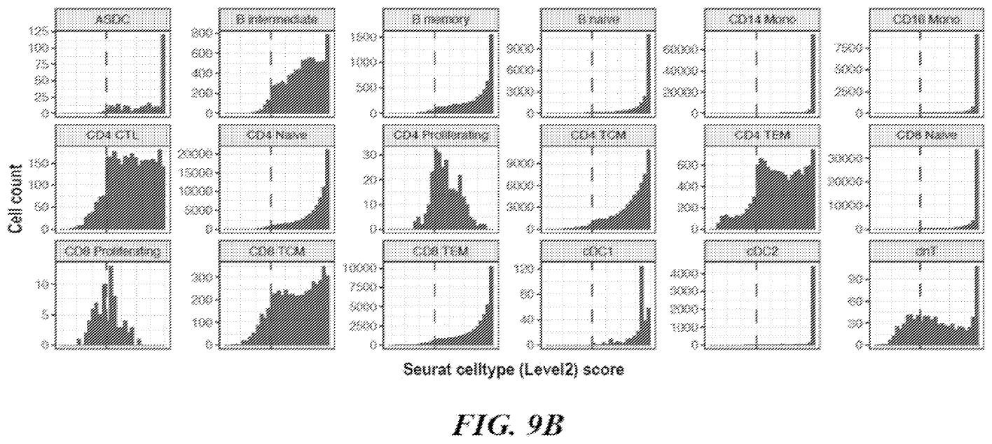

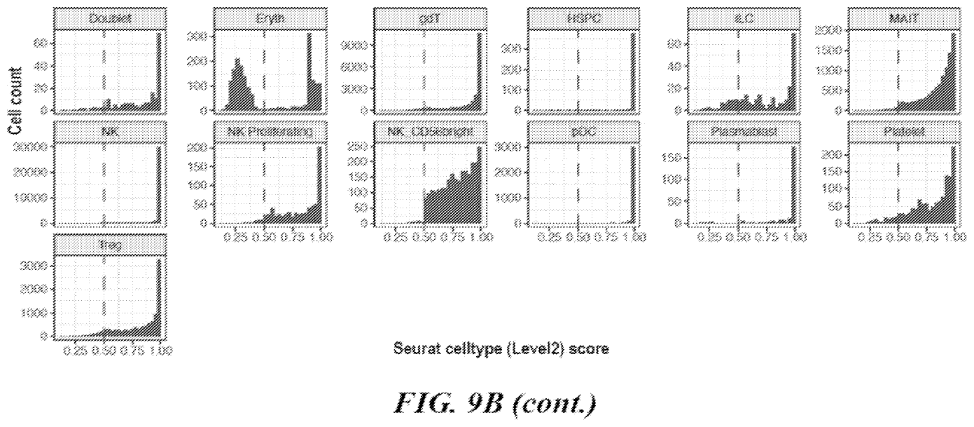

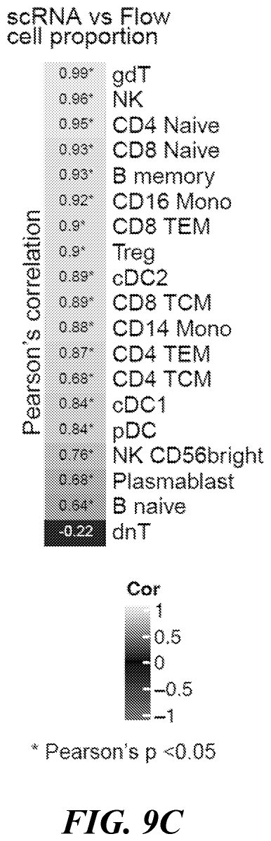

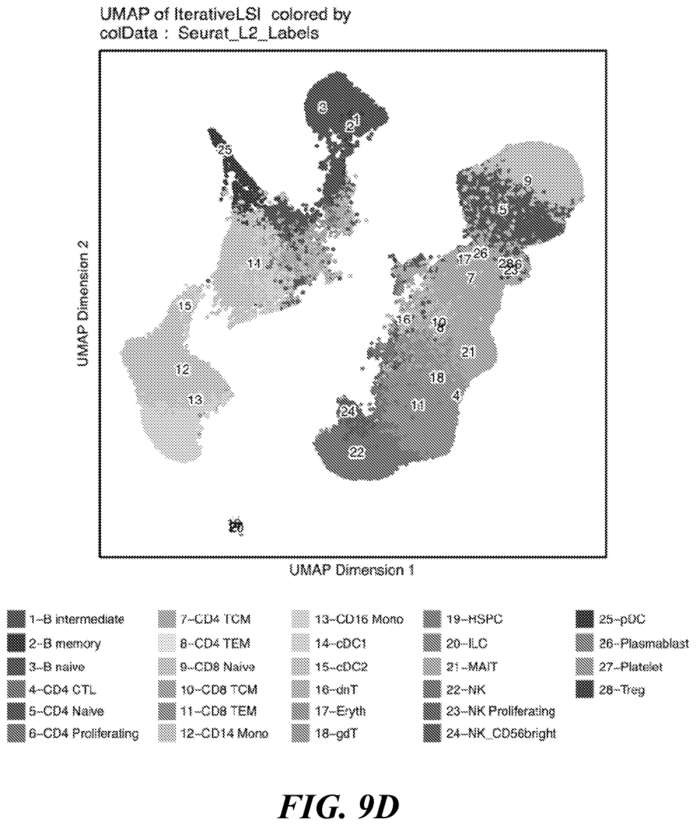

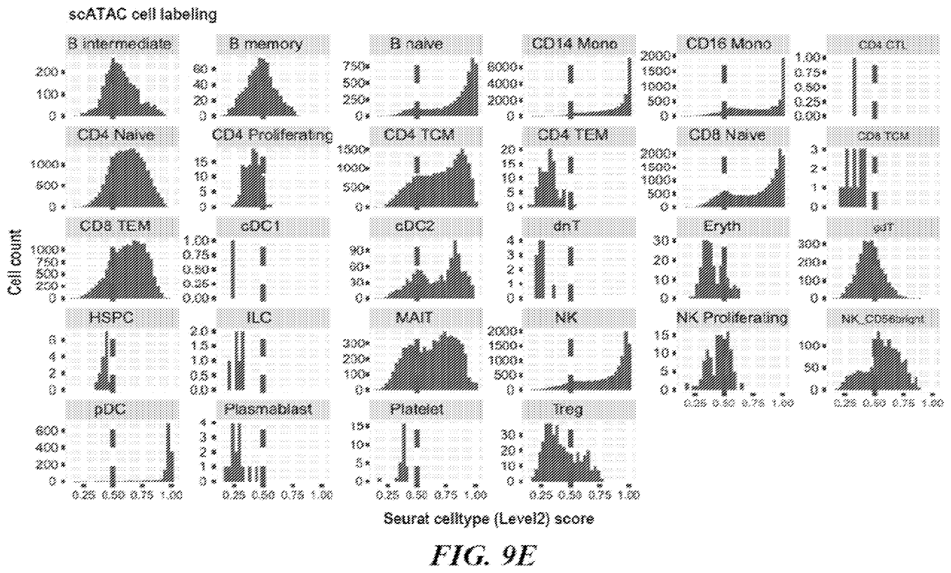

We retrieved high quality scRNA-seq data of 472,464 cells and labeled them to 31 different cell types using Seurat V216 (FIGS. 9A-9B, Table 4A). Among the nineteen overlapping cell types identified by both scRNA-seq and flow cytometry, the corresponding cell frequencies as measured by the two data modalities were highly correlated (p<0.05 on Pearson correlation) except for those of double negative T (dnT) cells (FIG. 9C). Unless specified otherwise, we filtered out low frequent cell types (average frequency <0.5%) and kept 19 out of the 31 cell types for downstream analysis (Table 4B). We also kept only 11,191 genes that had an average (across timepoints) expression of 0.1 or higher in at least one cell type of one donor.

scATAC-seq data was analyzed using the ArchR21 package. We observed 294,623 peaks in 135,566 cells after removing doublets. Cells were labeled to 28 different cell types using genescore matrix as implemented in ArchR (FIGS. 9D-9E). We noticed the labeling scores on scATAC-seq data were much lower than the corresponding scores on scRNA-seq data, likely reflecting the challenge in cell labeling on scATAC-seq data. We filtered out low quality cells (labeling score <0.5), removed cell types having less than 50 remaining cells, and kept 14 out of the 28 cell types for downstream analysis (Table 4B). We also kept only 24,769 genes that had an average (across timepoints) gene score of 0.1 or higher in at least one cell type of one donor.

In addition to our own data, we also evaluated PALMO performance against six external omics datasets of diverse complexities, different sample types and/or different technical platforms (Table 1B). More examples of PALMO usage beyond those presented here can be found in PALMO vignettes (https://github.com/aifimmunology/PALMO/blob/main/Vignette-PALMO.pdf), including performance on unbalanced data, data with replicates, and data of a single donor with multiple timepoints.

### Example 3: Application of VDA to Assess Sources of Variations

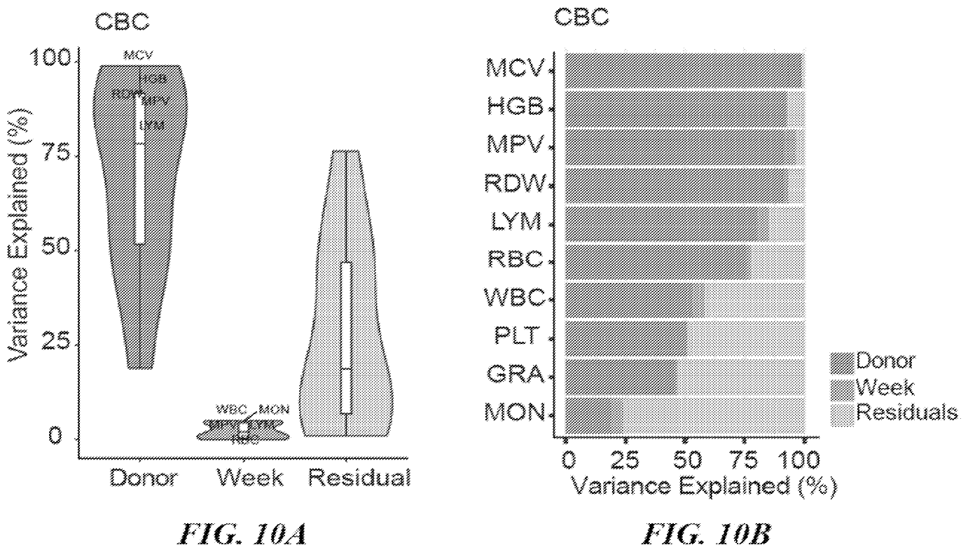

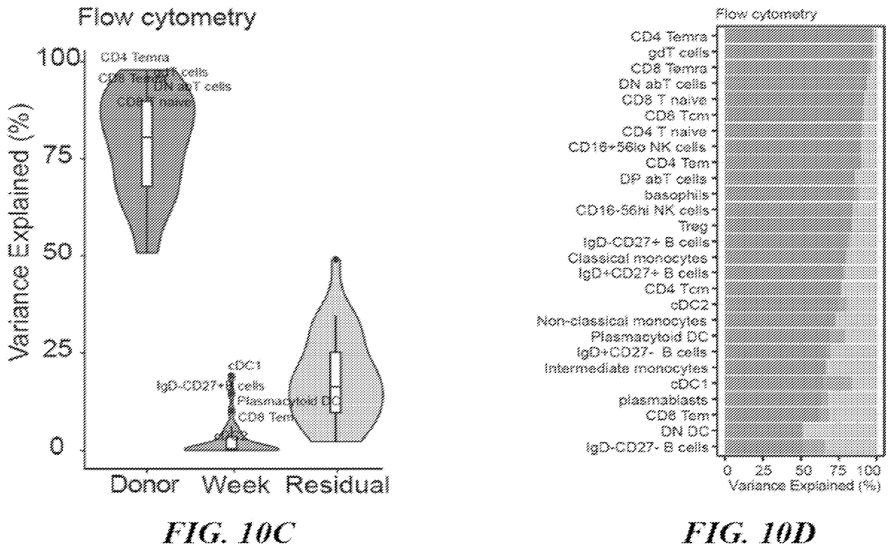

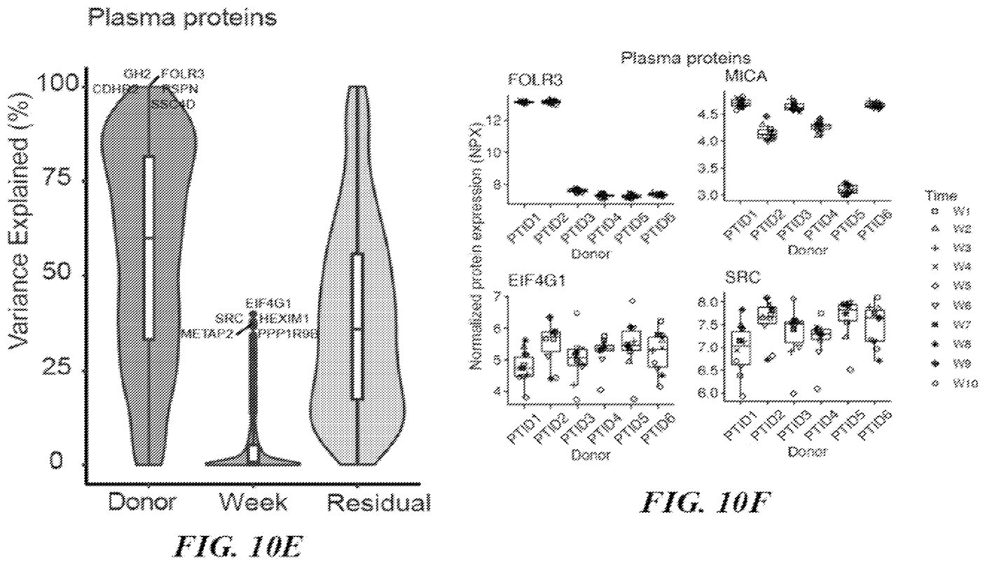

We applied VDA to evaluate inter- and intra-donor variations in our bulk data (CBC, PBMC frequencies from flow cytometry, and plasma proteomics data), using donor and week (timepoint) as factors of interest. CBC measurements showed strong inter-donor variations and minuscule intra-donor variations (FIGS. 10A-10B). PBMC frequencies from flow cytometry showed very strong inter-donor variations (FIGS. 10C-10D) with intra-class correlation (ICC) ranging from 51% (IgD CD27-B cells) to 98% (CD4 Temra: CD4+ effector memory T cells re-expressing CD45RA). In comparison, the highest ICC on intra-donor variations was 19% (cDC1: conventional type 1 dendritic cells). Plasma proteins followed a similar trend with some exceptions (FIGS. 10E-10F, Table 3A). Inter-donor variations of 621 (60%) out of the 1,042 quantified proteins contributed more than 50% to the corresponding total variance. Only 75 proteins (7%) had more intra-donor variation than inter-donor variation, but none contributed more than 50% to the total. A previous study identified 155 proteins having high inter-donor variations, 81% (126) of which overlapped with the 621 inter-donor variable proteins.

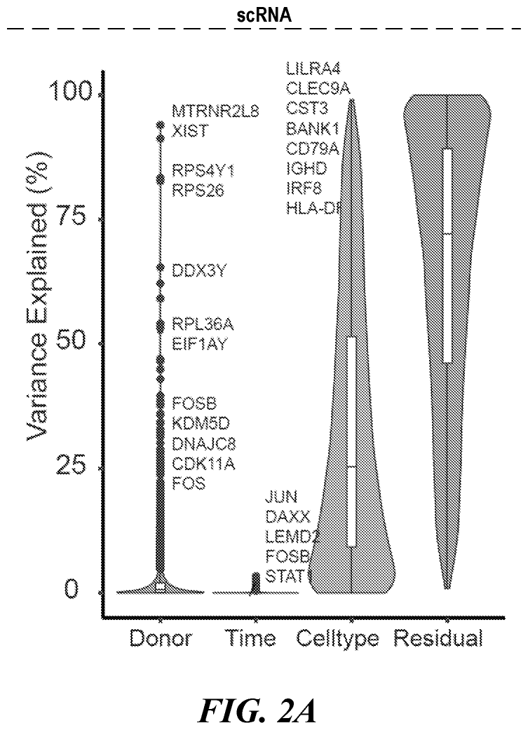

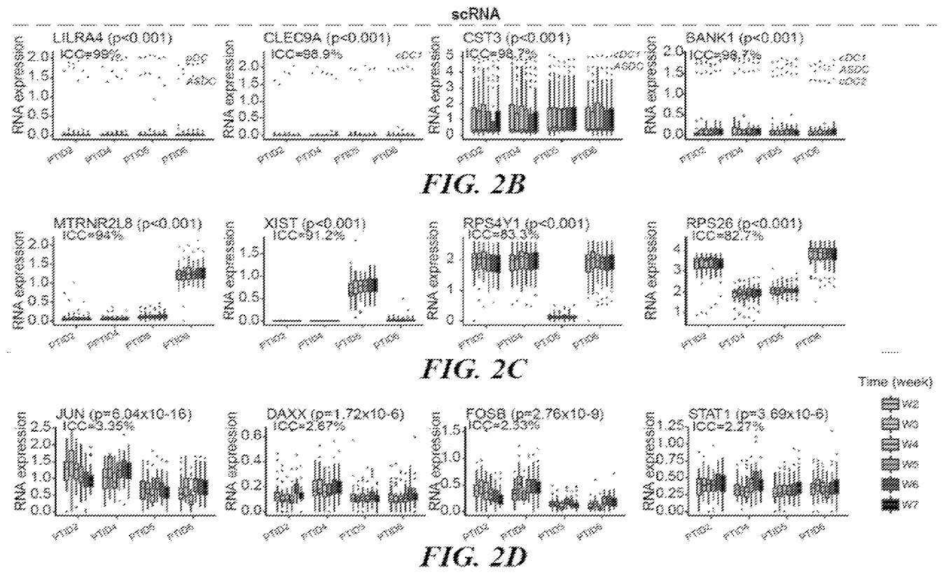

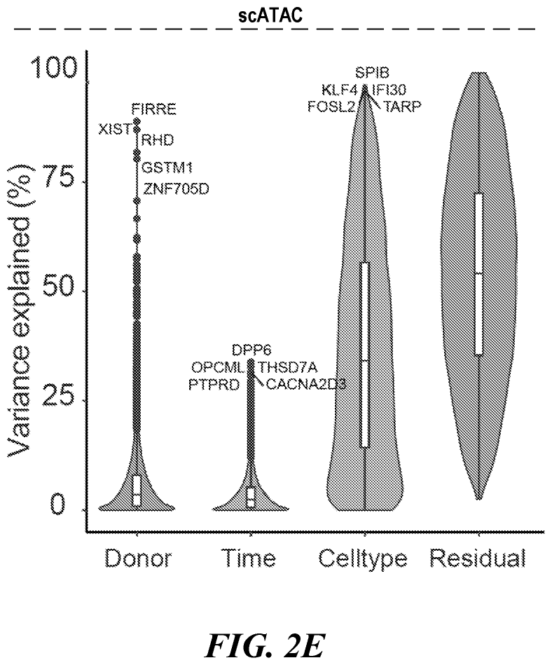

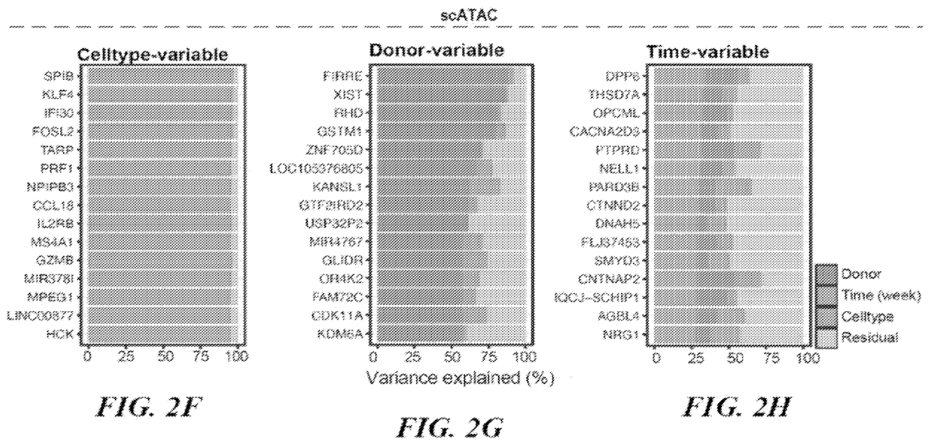

We added cell type as a factor of interest in the VDA of our scRNA-seq and scATAC-seq data. Inter-cell-type variations were more prominent than inter- and intra-donor variations in both single-cell data modalities. Based on our scRNA-seq data, 10, 0, and 4,384 genes had more than 50% of total variance from inter-donor, intra-donor, and inter-cell-type variations, respectively (FIG. 2A). Nine of the top ten inter-cell-type variable genes (ICC: 98-99%, FIG. 2B) have known immune functions (Table 4C). The top gene, LILRA4, is predominantly expressed in plasmacytoid dendritic cells (pDCs) and prevents pDCs from overblown reaction to viral infections. Six of the top ten inter-donor variable genes (ICC: 53-94%, FIG. 2C) are linked to the X or Y chromosome and seven of them showed differential expression between ovary and prostate/testis, reflecting the sex difference between male and female donors. Contributions from intra-donor variations to the total variance were small (ICC ≤3%, FIG. 2D), indicating the immune systems of the four healthy donors were quite stable over the study period.

The VDA results on our scATAC-seq data, using genescore matrix, showed similar trends as that on our scRNA-seq data (FIG. 2E). A total of 33, 0, and 7,847 genes had more than 50% of total variance from inter-donor, intra-donor, and inter-cell-type variations, respectively. All the top ten inter-cell-type variable genes (ICC: 95-97%, FIG. 2F) have known immune functions (Table 3D). The top gene, SPIB, is an enhancer regulating pDC development. Among the top ten inter-donor variable genes (ICC: 58-89%, FIG. 2G), XIST, ZNF705D, GTF2IRD2, and USP32P2 have differential expression between ovary and prostate/testis; RHD encodes a key protein in the Rh blood group system; and GSTM1 belongs to a highly polymorphic supergene family and affects heterogeneous response to toxicity. These genes appeared to capture more diverse types of differences among donors than their counterparts from scRNA-seq data. The ICCs of the top five intra-donor variable genes (ICC: 32-34%, FIG. 2H) were about 10-fold higher than that of the corresponding top gene, JUN, by scRNA-seq data, suggesting chromatin accessibility might be more sensitive to biological changes than gene expression.

variancePartition was previously developed to study variations in gene expression data and can be applied to longitudinal omics data for the same purpose. VDA generated almost identical results as variancePartition on two tested datasets after removing missing values (FIGS. 11A-11B), which was needed to run variancePartition but not VDA.

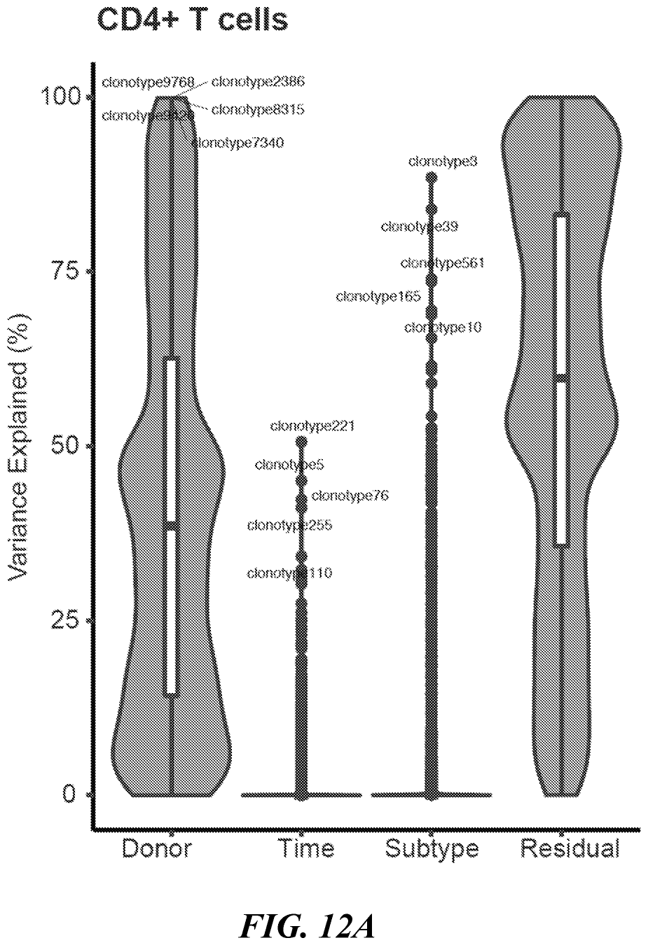

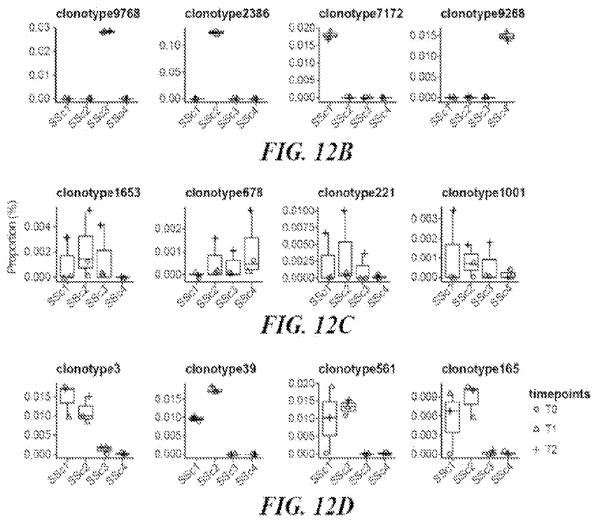

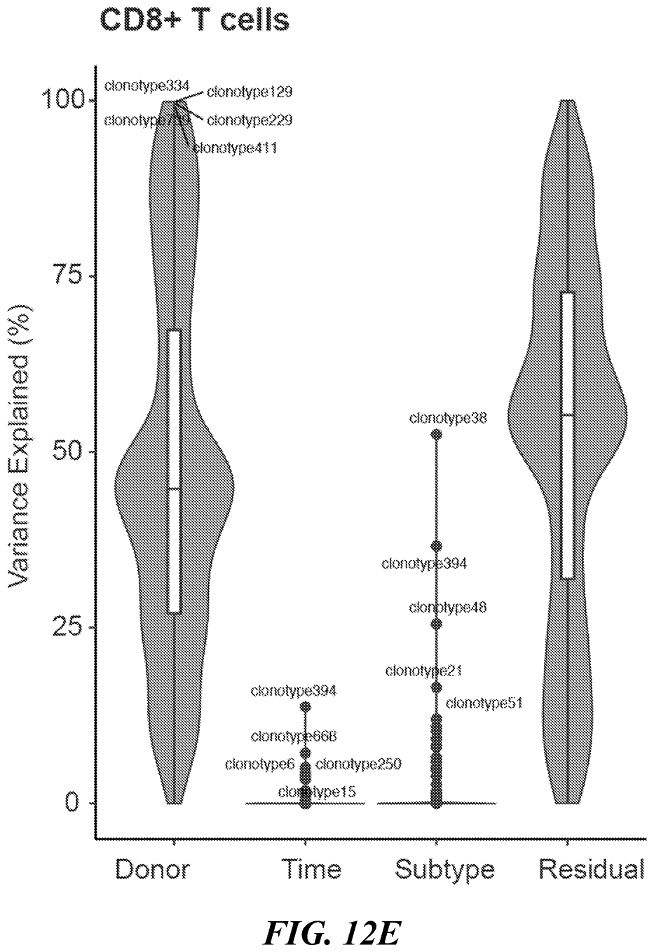

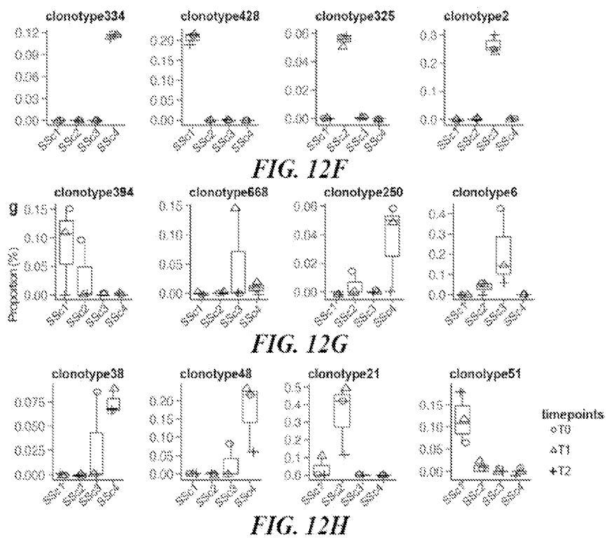

VDA can be used to study T cell receptor (TCR) repertoires. Previously sorted CD4+ and CD8+ non-naive T cells were isolated from PBMC samples of four systemic sclerosis (SSc) donors and analyzed to obtain sequencing data of TCR β-chains. The data was originally analyzed using tcR20, which was developed specifically for TCR data with functions either providing sample-level views on the whole repertories or treating clonotype data as binary (present or absent). We downloaded the TCRβ data (GSE156980) and calculated the frequency of unique clonotypes from both CD4+ and CD8+ T cells. A total of 288,597 unique clonotypes were obtained from CD4+ T cells and 11,739 from CD8+ T cells, respectively. We treated the clonotype data as continuous and used donor, time, and subtype (limited SSc versus diffuse SSc) as factors of interest in VDA. We identified from CD4+ T cells 6,625, 3, and 41 clonotypes having more than 50% of total variance from inter-donor, intra-donor, and inter-subtype variations, respectively (FIGS. 12A-12D). The corresponding counts from CD8+ T cells were 650, 0, and 1 (FIGS. 12E-12H). As illustrated in FIGS. 12B and 12F, many inter-donor variable clonotypes were donor-specific and stable over time, making them potential candidates responsible for SSc pathogenesis. The identification of inter-subtype variable clonotypes (FIGS. 12D, 12H) is interesting since some of them might be specific to either limited SSc or diffuse SSc. VDA provided novel insights on the TCR data, which was not presented in the original study.

### Example 4: Application of CVP to Evaluate Longitudinal Stability

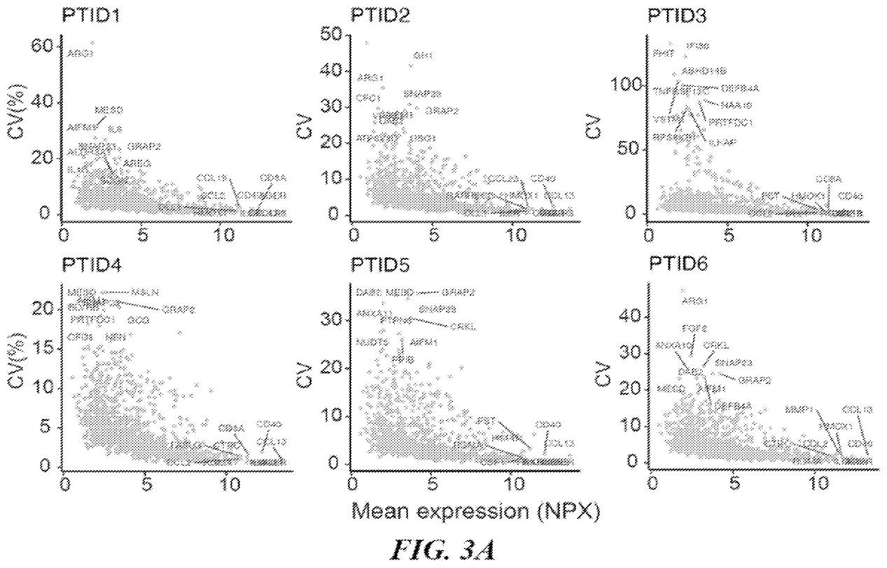

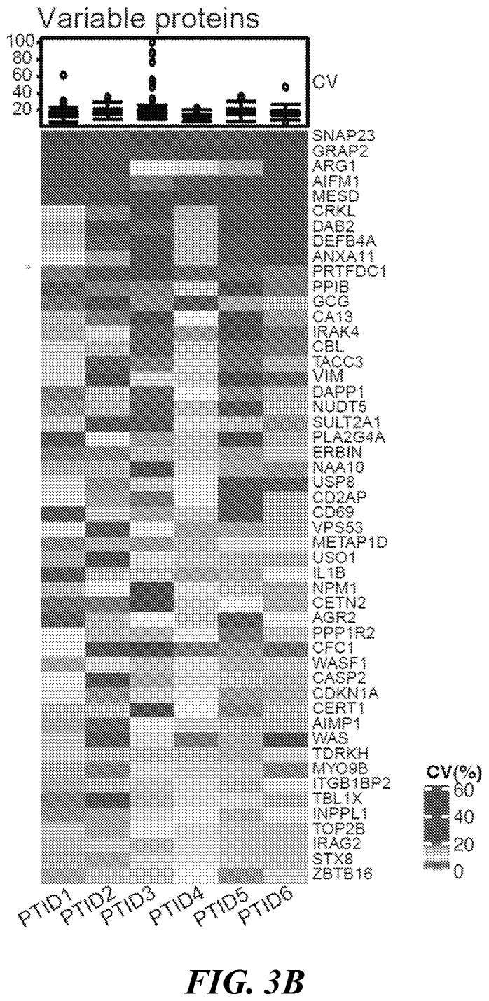

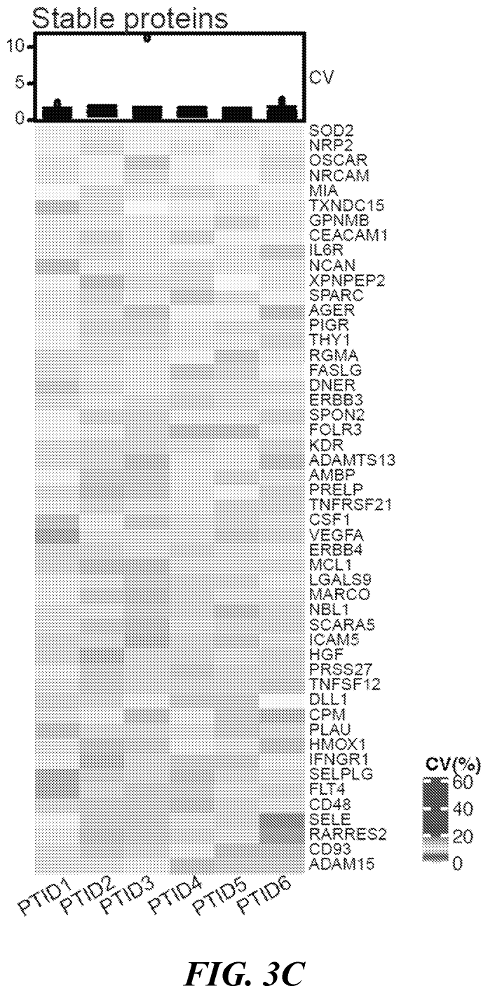

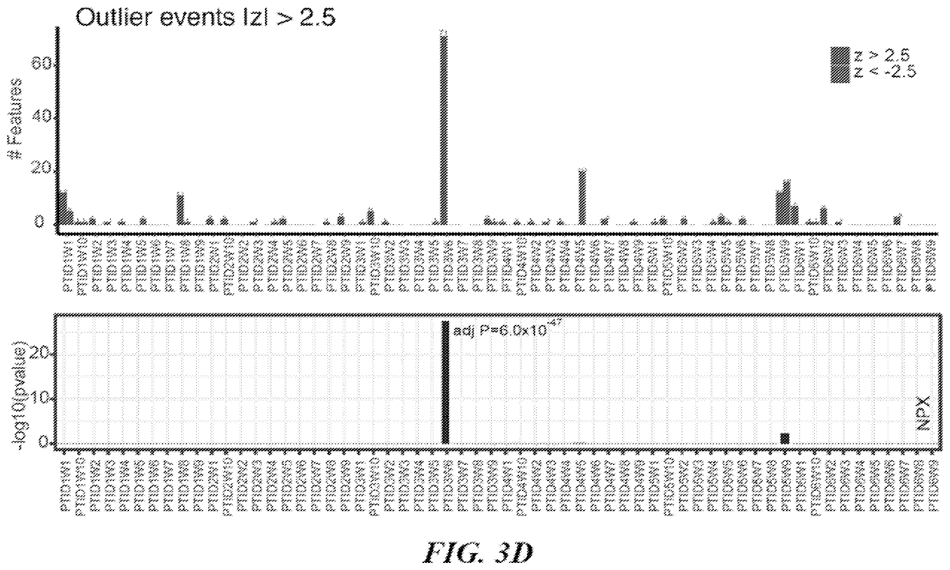

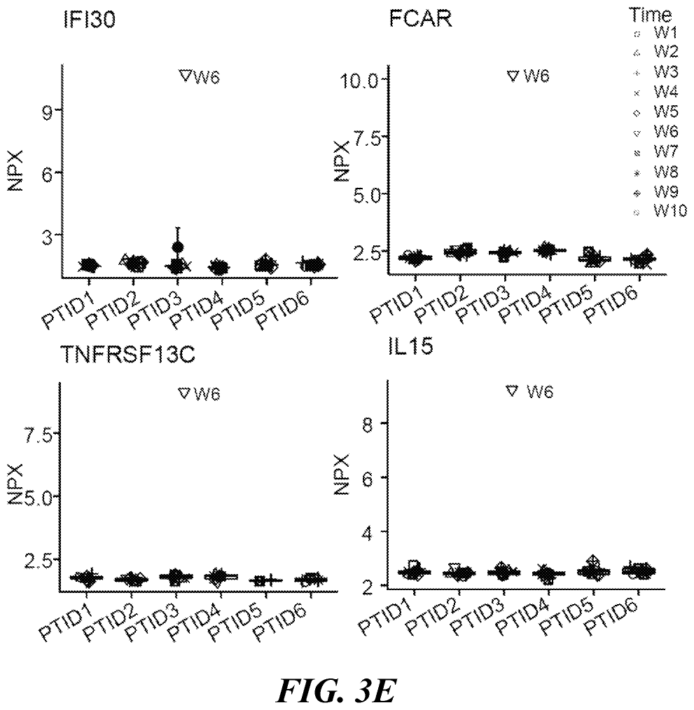

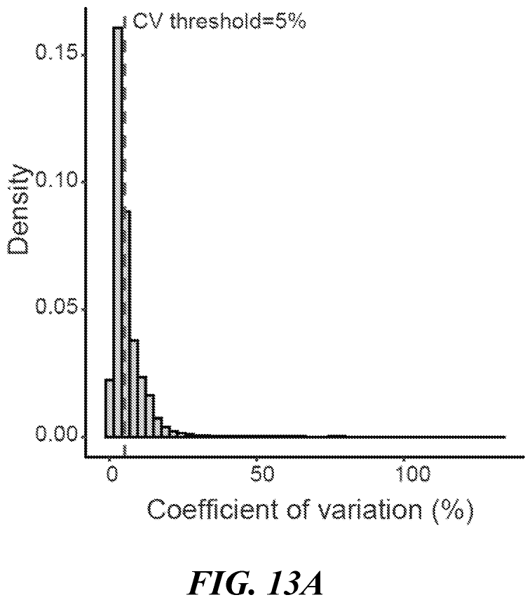

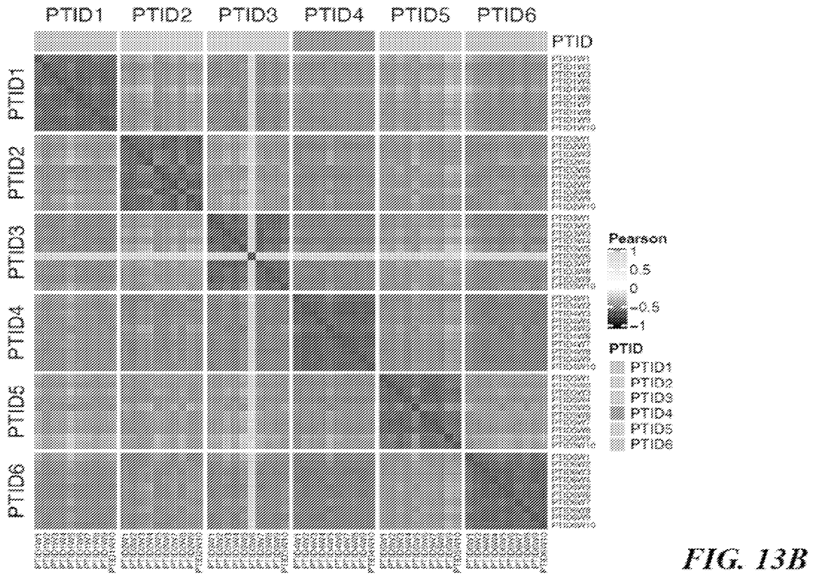

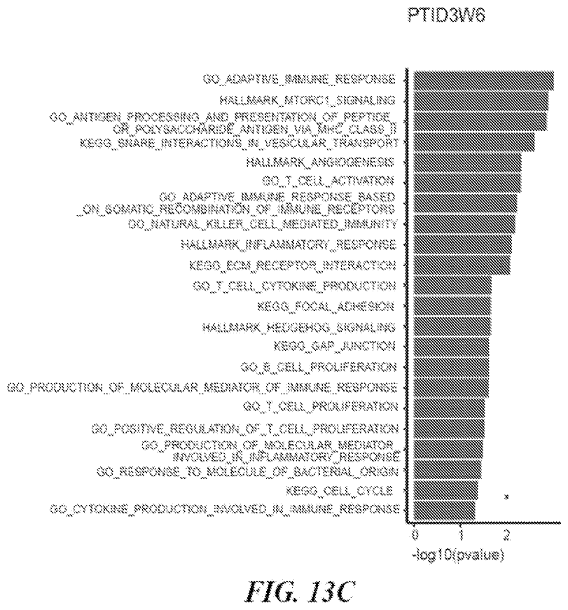

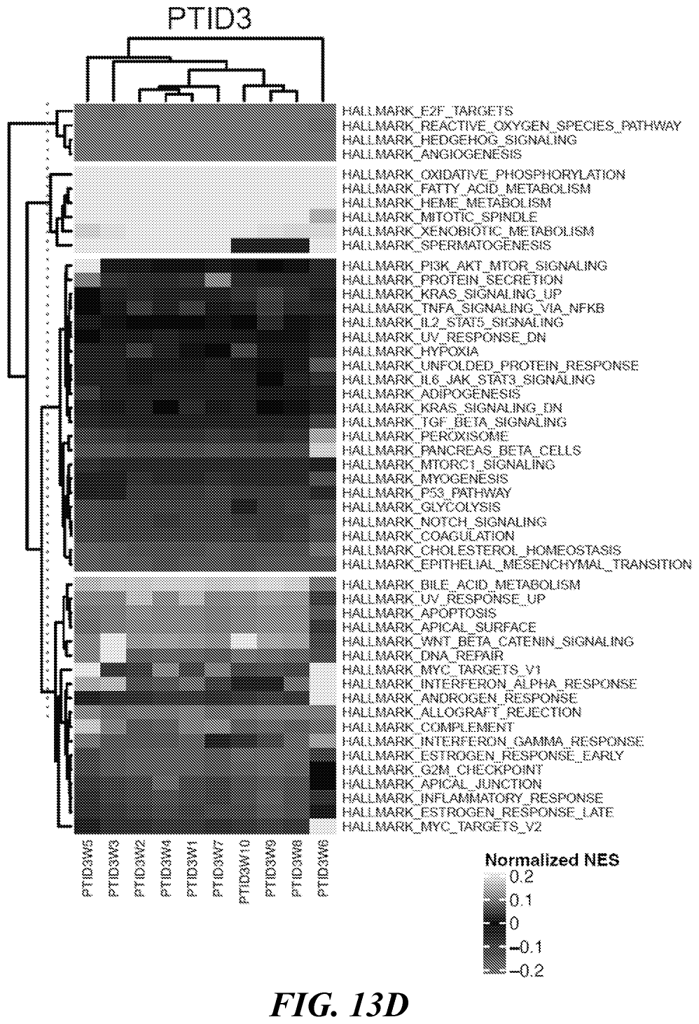

We applied CVP to identify longitudinally stable and variable proteins from our proteomics data (FIG. 3A). The distribution of median CV (among donors) peaked near 5% (FIG. 13A), which we used as a cut-off to separate variable (median CV >5%) and stable (median CV<5%) proteins (Tables 3B-3D). A total of 413 proteins were longitudinally variable, among which SNAP23, GRAP2, ARG1, AIFM1, and MESD had the highest median CV (24.6-27.7%, FIG. 3B). Such moderate CV values are consistent with the observed low intra-donor variations by VDA. A total of 629 proteins were longitudinally stable, among which SOD2, NRP2, OSCAR, NRCAM, and MIA had the lowest median CV (0.6-0.8%, FIG. 3C). These stable proteins may be interesting biomarker candidates if they change under some disease conditions. They can also be used to bridge proteomics data of different experimental batches.

### Example 5: Application of ODA to Discover Possible Abnormal Events

We noticed that proteomics data of donor PTID3 exhibited higher CV values than those of other donors (FIG. 3A) and weaker intra-donor correlations at week 6 than at other weeks (FIG. 13B). We applied ODA to check whether donor PTID3 had an abnormal event at week 6. We selected |z|>2.5 as the criterion for outliers so that just above 1% of all quantifiable proteins are expected to be outliers. More accurately, we expected 1.24% of proteins (i.e., 19 proteins per donor per time point), to be outliers by chance. A total of 71 outlier proteins were identified at Week 6 on donor PTID3 (adj p=6.0×10⁻⁴⁷, FIGS. 3D-3E, Tables 3E-3F). Eight of the top ten proteins having the highest z scores (2.84-2.85) play important roles in immune response and immunity (Table 3G). Gene set enrichment analysis (GSEA) revealed the outlier proteins were enriched in immunological processes, such as adaptive immune responses, antigen processing and presentation via major histocompatibility complex (MHC) class II, T cell activation, etc. (FIG. 13C). Single-sample GSEA (ssGSEA) on all PTID3 samples identified Week 6 as an outlier and revealed increased activity at Week 6 in important immune processes (FIG. 130), including MYC targets (v1 and v2), interferon-alpha and gamma responses, androgen response, pancreas beta cells, and peroxisome. Although further validation is required, these results suggest the possible occurrence of an immunological perturbation event (such as infection) experienced by PTID3 at week 6. Such outlier phenotypes can be obscured by analyses focusing on differences between sample groups.

### Example 6: Application of SPECT to Reveal Diverse Gene Stability Patterns

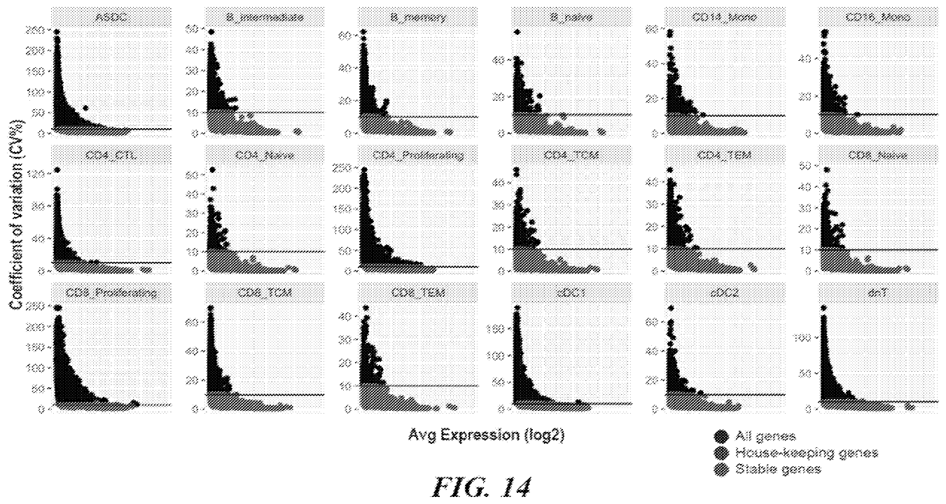

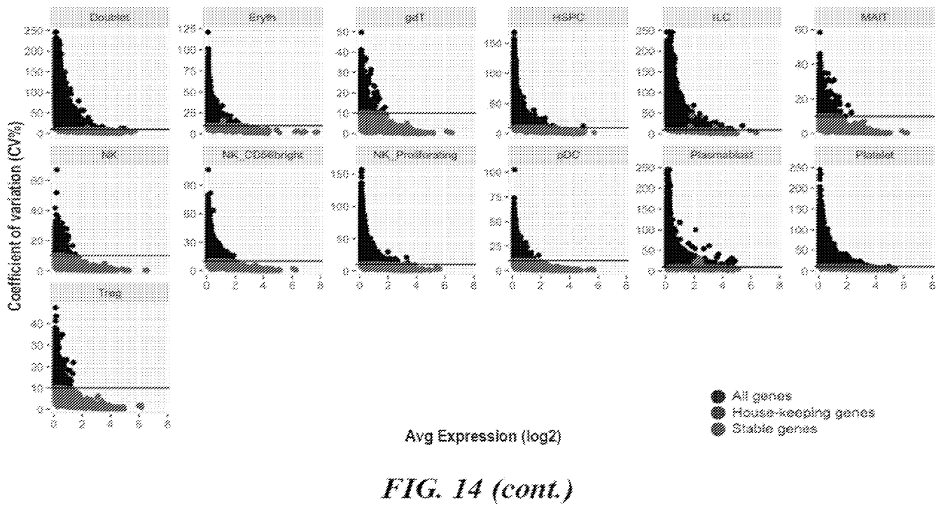

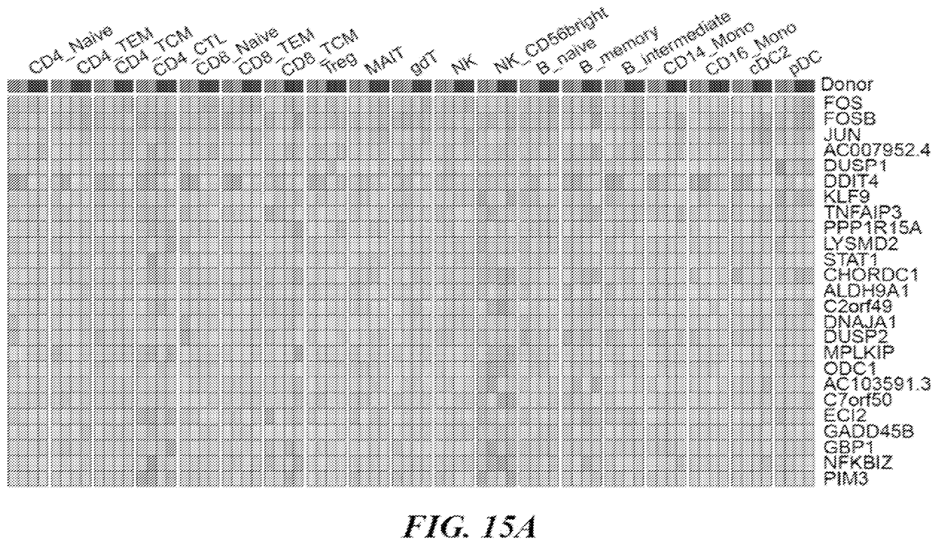

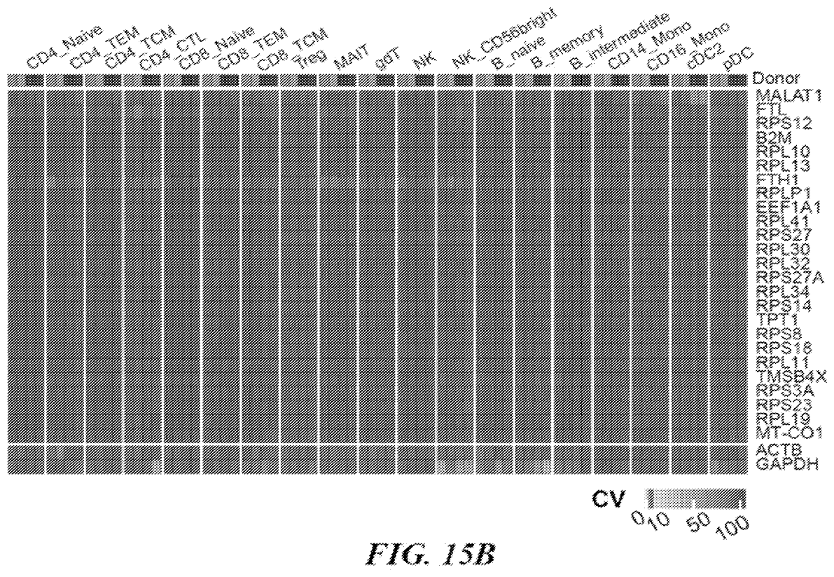

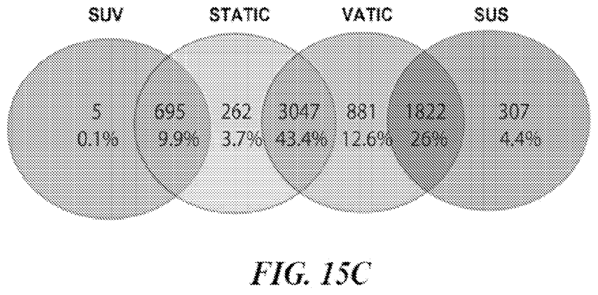

We applied SPECT to analyze our scRNA-seq data. Noticing the two well-known housekeeping genes, ACTB and GAPDH, had CVs (across timepoints) just above 10% in some cell types (FIG. 14), we used a CV cut-off of 10% to separate longitudinally variable (CV >10%) or stable (CV<10%) genes in individual cell types of individual donors. We then counted how many times individual genes were variable and/or stable in the 76 combinations between donor (n=4) and cell type (n=19). A gene was denoted as super variable (SUV) or super stable (SUS) if it was variable or stable in at least 40 donor-cell type combinations. A gene was denoted as variable across time in cell-types (VATIC) or STATIC if it was variable or stable in at least one cell type across all donors but in less than 40 donor-cell type combinations. We identified a total of 700 SUV genes (FIG. 15A), 2,129 SUS genes (FIG. 15B), 5,750 VATIC genes, and 4,004 STATIC genes from the dataset. Since a gene can be consistently variable in one cell type and consistently stable in another, VATIC and STATIC genes are not mutually exclusive (FIG. 15C).

The SUV genes were enriched in 57 pathways, many of which are associated with cellular proliferation and activity (Table 4E). Eight of the top ten SUV genes (Table 4F) have distinct roles in gene regulation, including four transcription factors (FOS, FOSB, JUN, and KLF9), two phosphatases (DUSP1 and PPP1R15A), one regulator of mTOR pathway (DDIT4), and one inhibitor of NF-κB pathway (TNFAIP3). In comparison, the SUS genes were enriched in 501 pathways of rather diverse, basic cellular processes (Table 4G). Among the top ten SUS genes (Table 4H), five (RPS12, RPL10, RPL13, RPLP1, and RPL41) encode ribosomal proteins and two (FTL and FTH1) encode ferritin for iron storage. Many SUS genes are more stable than ACTB and GAPDH and may be good candidates for estimating batch effects in scRNA-seq data.

### Example 7: STATIC Genes as Potential Biomarkers for Cell Types or Biological Conditions

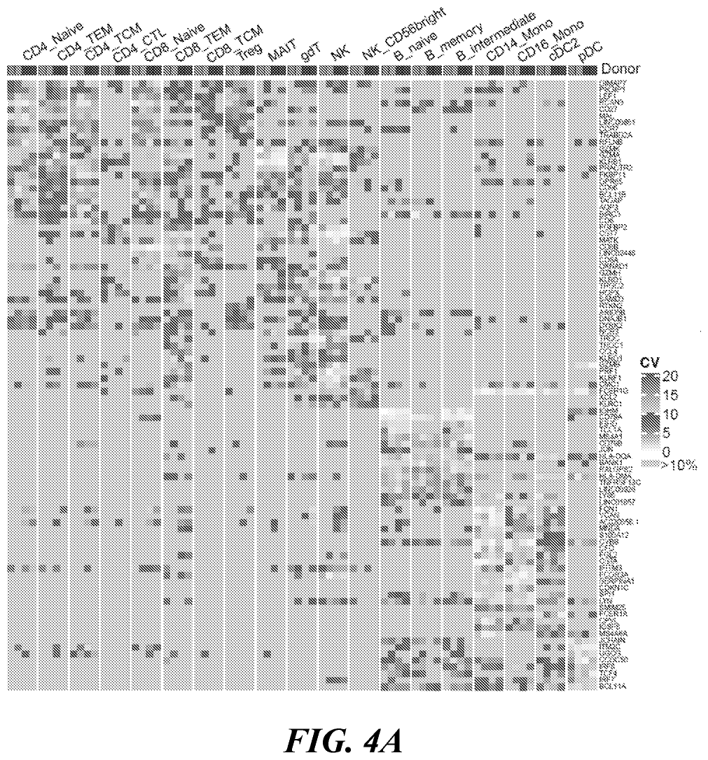

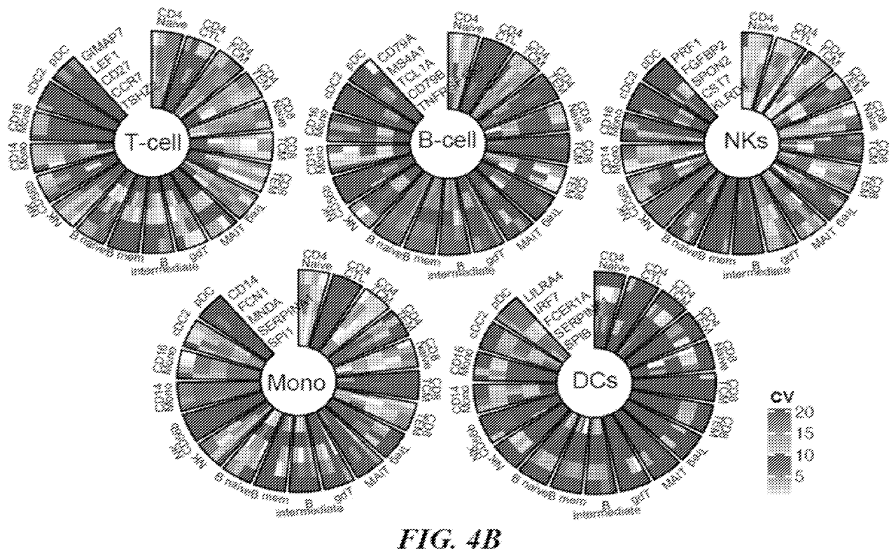

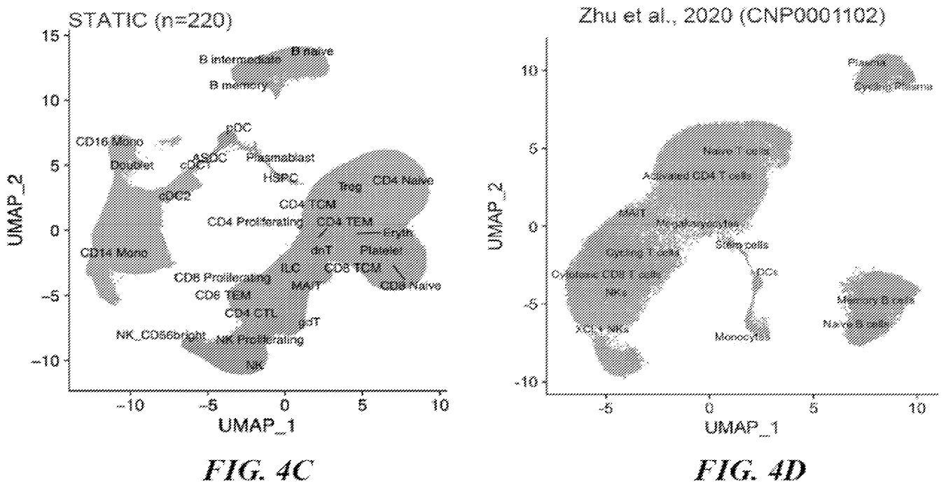

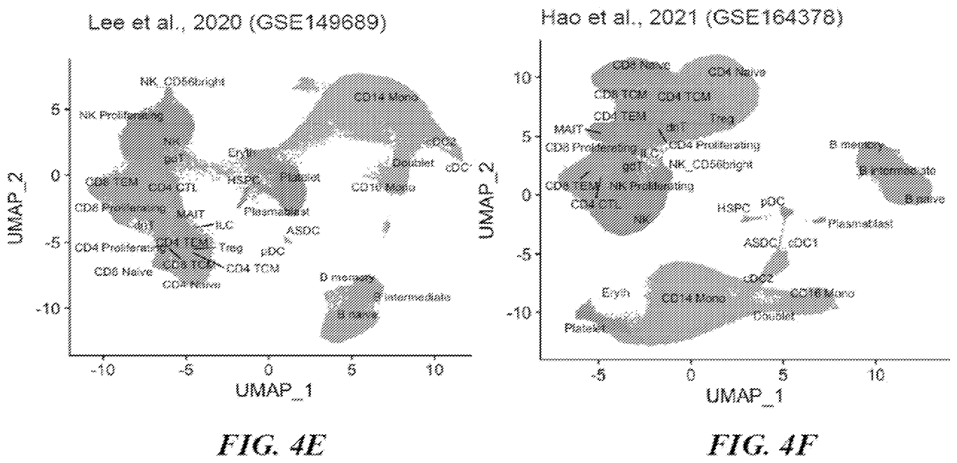

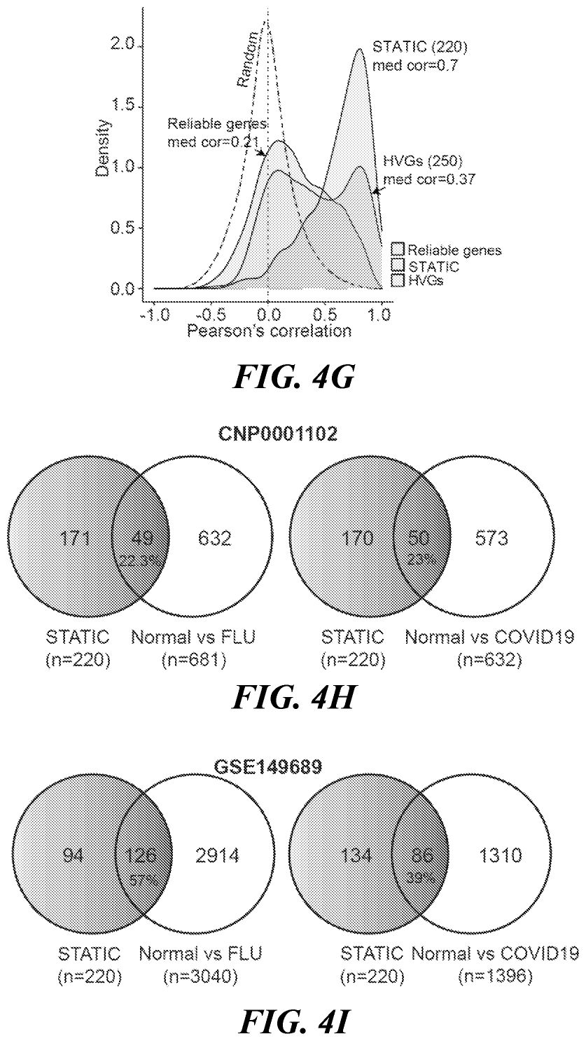

We collected up to 25 top STATIC genes from each cell type and obtained 220 unique genes (FIG. 4A, Table 5A). These 220 STATIC genes (also referred to as the STATIC 220 genes) are enriched in pathways such as innate (adj p=1.43×10⁻⁹) and adaptive (adj p=1.33×10⁻⁹) immune response, allograft rejection (adjusted p=3.06×10⁻¹⁶), lymphocyte mediated immunity (adj p=3.72×10⁻⁸), myeloid mediated immunity (adj p=2.71×10⁻⁵), B/T cell proliferation (adj p<1.46×10⁻³), acute inflammatory response (adj p=7.48×10⁻³), and hematopoietic cell lineage (adjusted p=2.44×10⁻⁴) (Table 5B). Examples of top STATIC genes for major cell types were shown in FIG. 4B, including: GIMAP7, LEF1, CD27, CCR7, and TSHZ2 for T cells; CD79A, MS4A1, TCL1A, CD79B, and TNFRSF13C for B cells; PRF1, FGFBP2, SPON2, CST7, and KLRD1 for natural killer (NK) cells; CD14, FCN1, MNDA, SERPINA1, and SPI1 for monocytes; and LILRA4, IRF7, FCER1A, SERPINF1, and SPI1 for dendritic cells (DCs). All these genes demonstrated cell type-specific stability patterns and have well-documented roles in the corresponding cell types (Table 5C).

We used the 220 STATIC genes as input features and projected PBMCs in our scRNA-seq data onto a two-dimensional Uniform Manifold Approximation and Projection (UMAP; FIG. 4C), which we refer to as sUMAP from now on. We also generated sUMAPs using the same 220 STATIC genes on three independent scRNA-seq datasets of PBMCs (FIGS. 4D-4F) in which cells were labeled as in the original studies. In all four cases, the 220 STATIC genes separated major cell types and most of their subtypes very well, suggesting that some STATIC genes are potentially good markers for cell types.

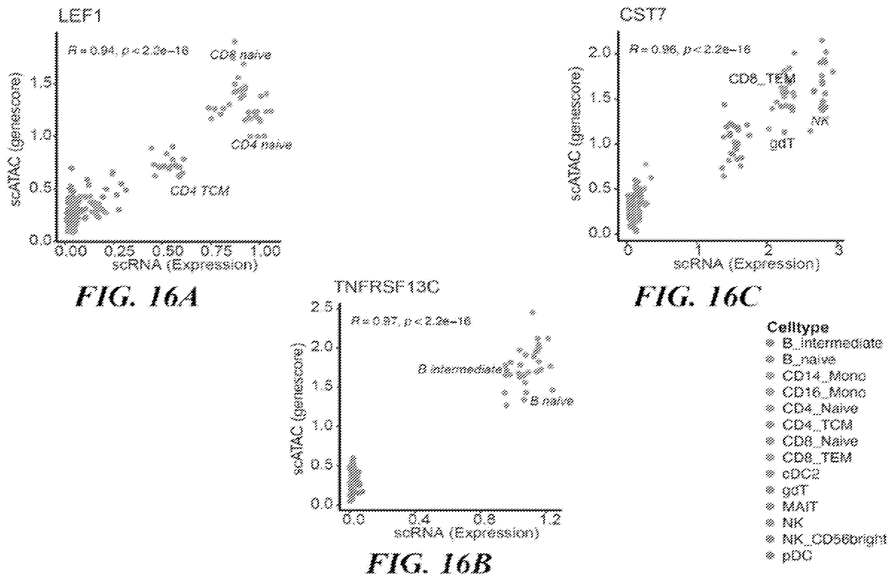

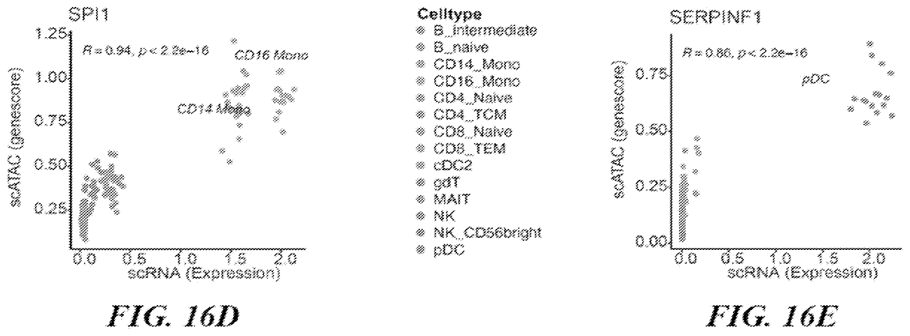

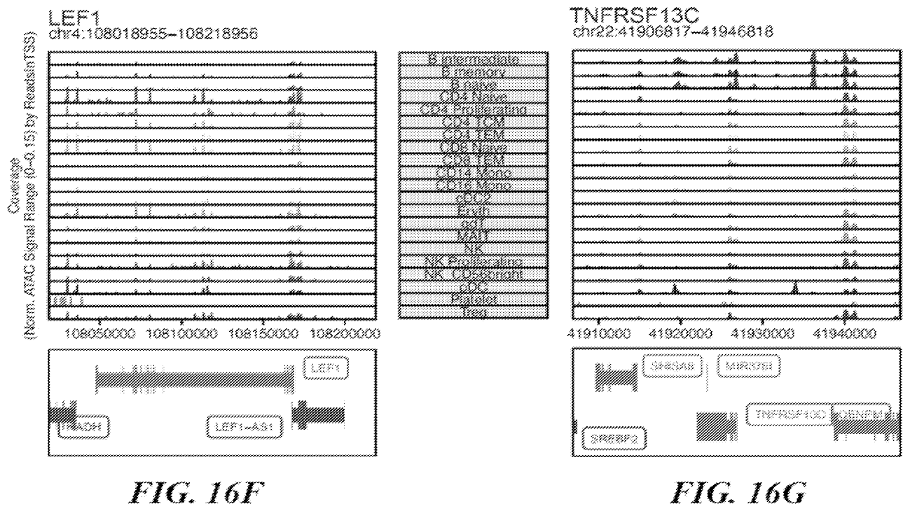

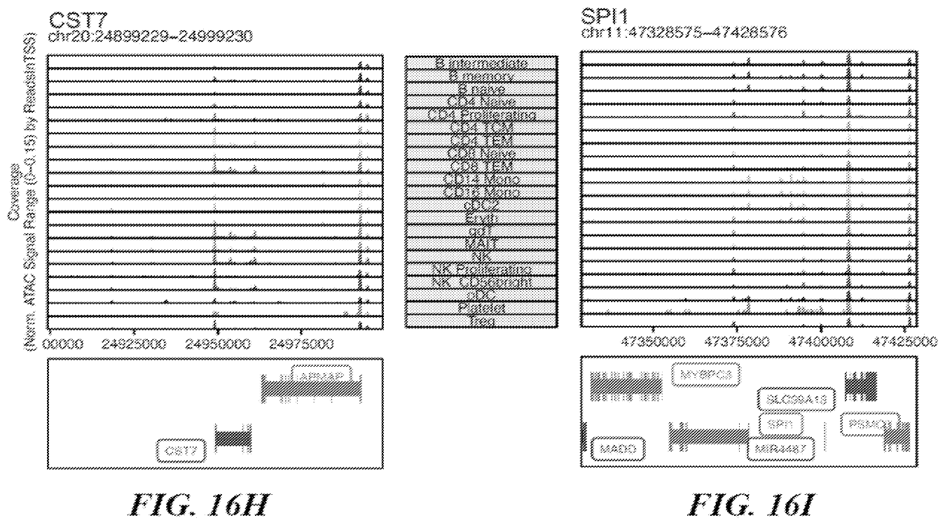

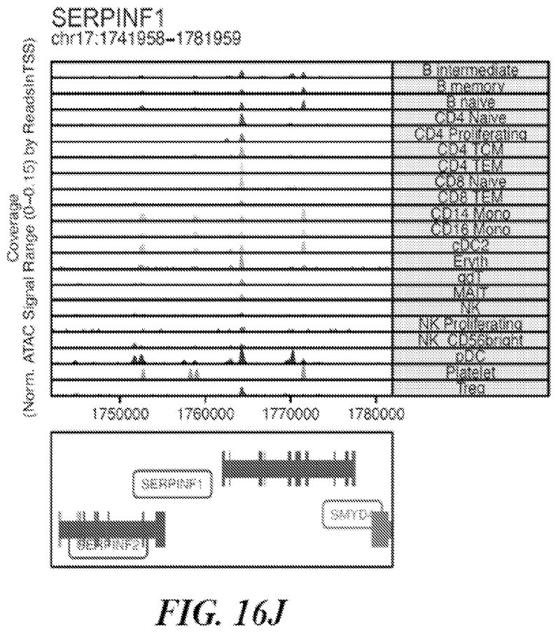

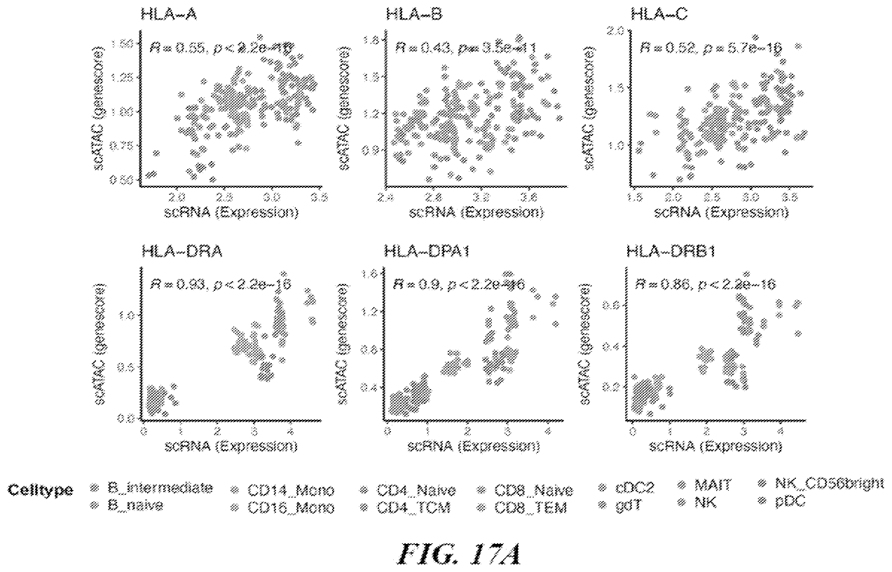

Gene scores are routinely computed from scATAC-seq data to infer expression of the corresponding genes and used to label cells in scATAC-seq data based on a scRNA-seq reference. We calculated the Pearson correlation between expression in scRNA-seq data and gene score in scATAC-seq data of the same genes across cell types and samples. Due to data sparseness, incomplete reference assembly, non-coding RNAs, and uncharacterized sequences, Pearson correlation could be calculated only on 10,611 (95%) of the 11,191 reliable genes (FIG. 4G). Interestingly, among genes with strong correlations (FIGS. 16A-16J), the correlation was mainly influenced by differences between cell types, which partially justifies the use of gene score for cell labeling on scATAC-seq data. Within individual cell types, the correlation however appeared to be poor across different samples, likely reflecting the complexity of gene regulation. Pearson correlation was obtained on 208 (95%) of the 220 STATIC genes with a median value of 0.70. In comparison, Pearson correlation was obtained on 232 (93%) of the top 250 highly variable genes (HVGs), which are widely used in dimension reduction on scRNA-seq data, with a significantly lower median value of 0.37 (p=2.2×10⁻¹⁶, Mann-Whiney test; Table SD). We randomly paired unrelated genes, calculated the corresponding correlations between expression and gene score, and found that the obtained distribution had a 95% upper confidence bound at R₀=0.399. Thus, any correlations below R₀ were not statistically better than those between random, unrelated gene pairs. A total of 7,255 (68%) out of the 10,611 reliable genes and 128 (55%) out of the 232 HVGs had a correlation below R₀, in comparison with 42 (20%) out of the 208 STATIC genes. To properly label cells in scATAC-seq data based on gene score approach, one should only use genes whose expression versus gene score correlations are above R₀. Some STATIC genes might be good candidates for this purpose.

We further investigated how the 220 STATIC genes fared as potential disease biomarkers. Previously, two studies applied scRNA-seq to analyze PBMCs of healthy controls (Normal) and of patients infected with either influenza (FLU) or SARS-CoV-2 (COVID-19). We reanalyzed the data using methods described in the original studies and identified differential expression genes (DEGs) distinguishing Normal versus FLU or Normal versus COVID-19. For simplicity, DEGs from individual cell types were combined when compared with the 220 STATIC genes. Out of the 18,824 genes measured in the first study (CNP0001102), 681 and 632 DEGs were identified for distinguishing Normal versus FLU and Normal versus COVID-19, respectively. The corresponding overlap with the STATIC genes was 49 for Normal versus FLU (hypergeometric p=4.8×10²6) and 50 for Normal versus COVID-19 (hypergeometric p=1.7×10⁻²⁸, FIG. 4H). A total of 33,538 genes were measured in the second study (GSE149689). A total of 126 STATIC genes (hypergeometric p=4.8×10⁻⁷⁴) overlapped with the 3,040 DEGs for Normal versus FLU while 86 STATIC genes (hypergeometric p=2.1×10⁻⁶¹) overlapped with the 1,396 DEGs for Normal versus COVID-19 (FIG. 4I). In all cases, the 220 STATIC genes were significantly enriched as DEGs, suggesting their potential for monitoring some disease conditions.

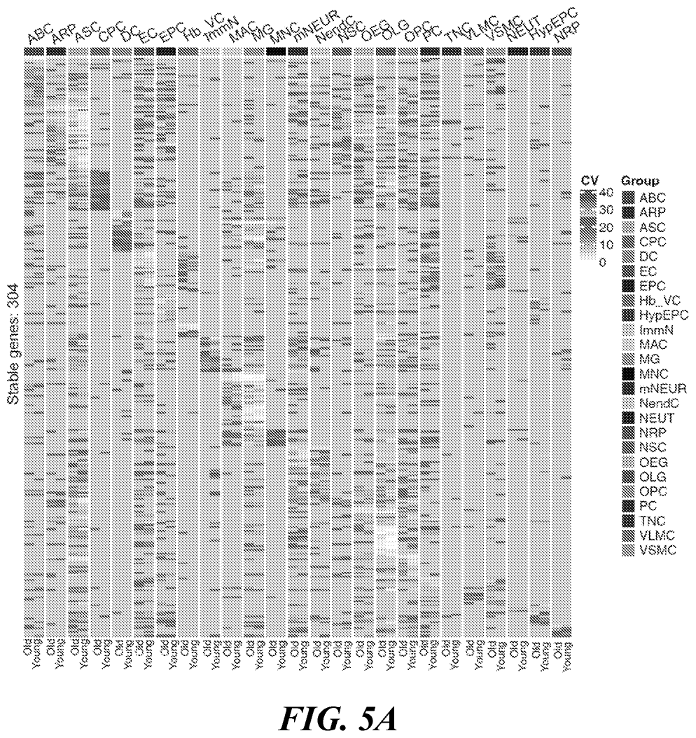

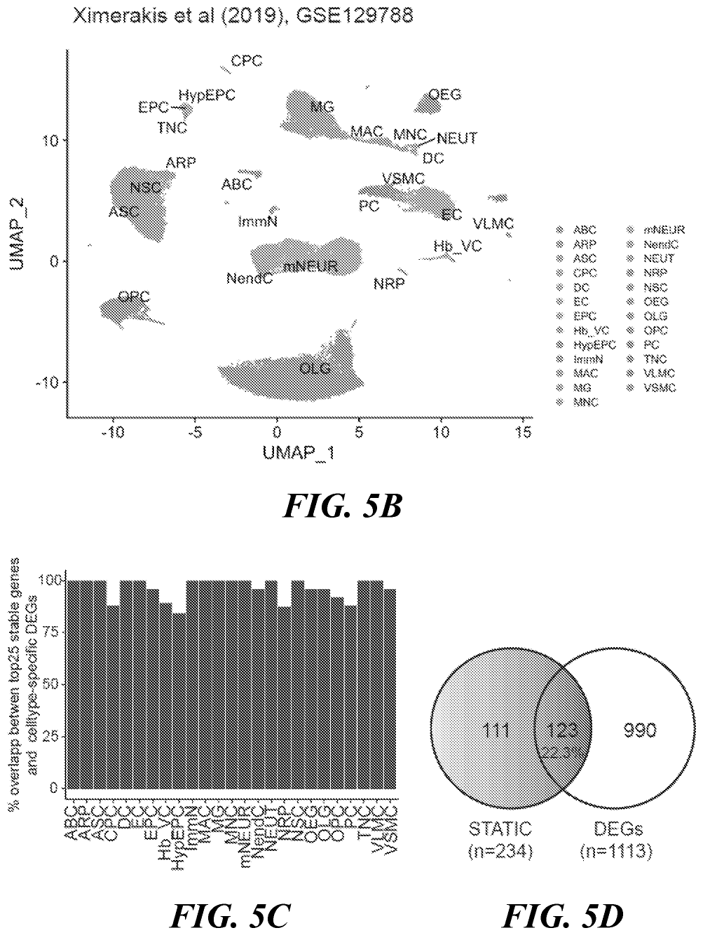

To illustrate that SPECT can handle scRNA-seq data of diverse sample types, we applied it to scRNA-seq data from a mouse brain study (GSE129788). In the study, scRNA-seq data was collected from brain tissues of eight young (2-3 months) and eight old (21-23 months) mice, from which 37,069 cells of high quality data were labeled to 25 cell types, 14,699 genes were detected, marker genes for each of the 25 cell types were collected, and 1,113 DEGs distinguishing young versus old mouse brains were identified from a subset of 15 cell types. The study was not longitudinal per se. We treated data from the eight samples of each age group as repeated measurements for the group, just like repeated measurements at different timepoints in a longitudinal study. Since SPECT does not utilize the ordering of timepoints, its usage to the data is justified. We collected up to 25 STATIC genes per cell type and obtained 304 unique genes from all 25 cell types (FIG. 5A, Table 6A). sUMAP, using these 304 STATIC genes, was able to separate the cell types as labeled in the original study very well (FIG. 5B). Out of the 304 STATIC genes, 299 genes were identified in the original study as marker genes for the corresponding cell types (FIG. 5C, Table 6B). From the 15 cell types having DEGs, we collected 234 STATIC genes that were significantly overlapped with the 1,113 young versus old DEGs (n=123, hypergeometric p=6.2×10⁻⁷⁷, FIG. 5D). These results further demonstrated that some STATIC genes are good markers for cell types or biological conditions in the mouse brain study.

### Example 8: Circos Plots to Reveal Stability Patterns of Protein Families

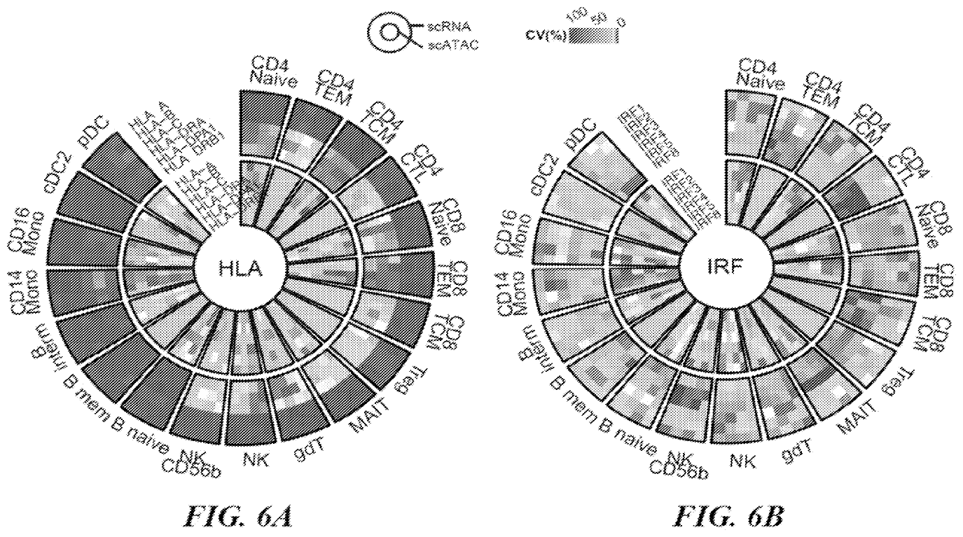

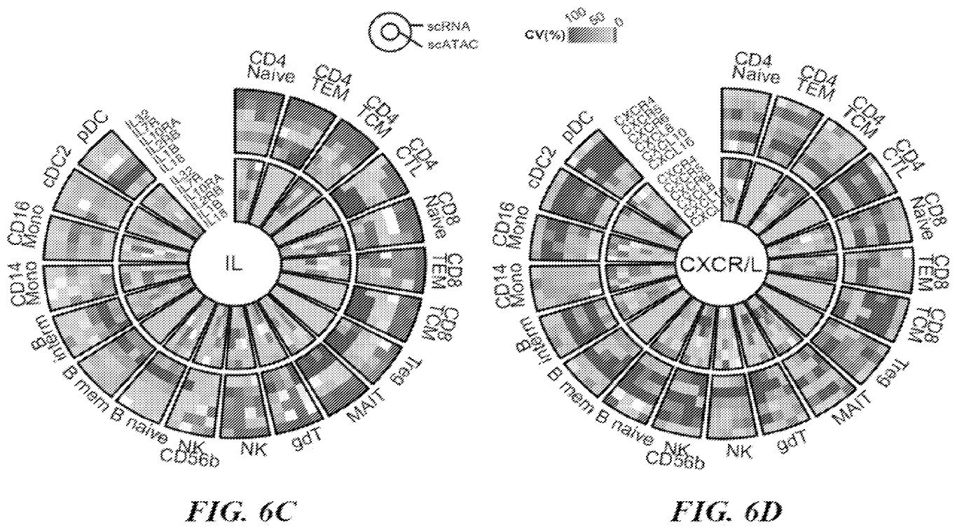

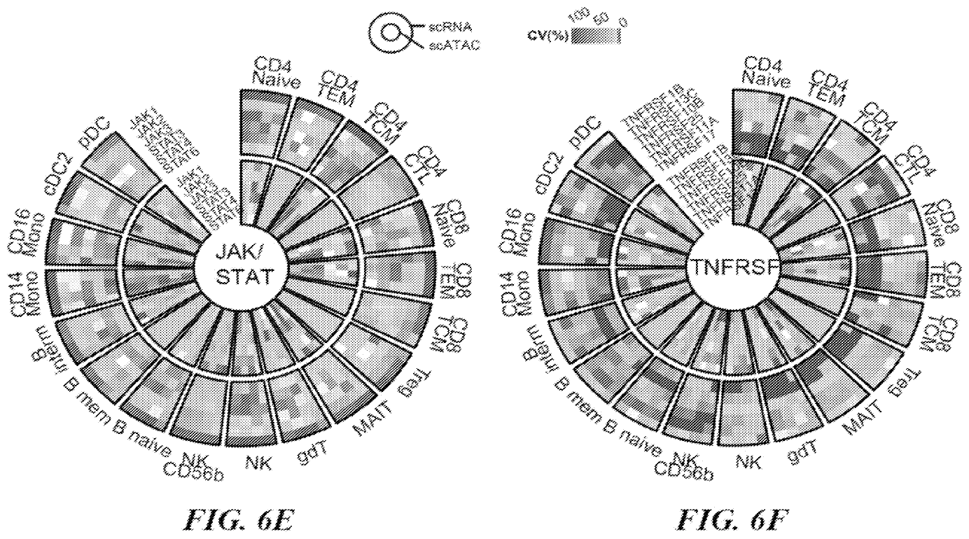

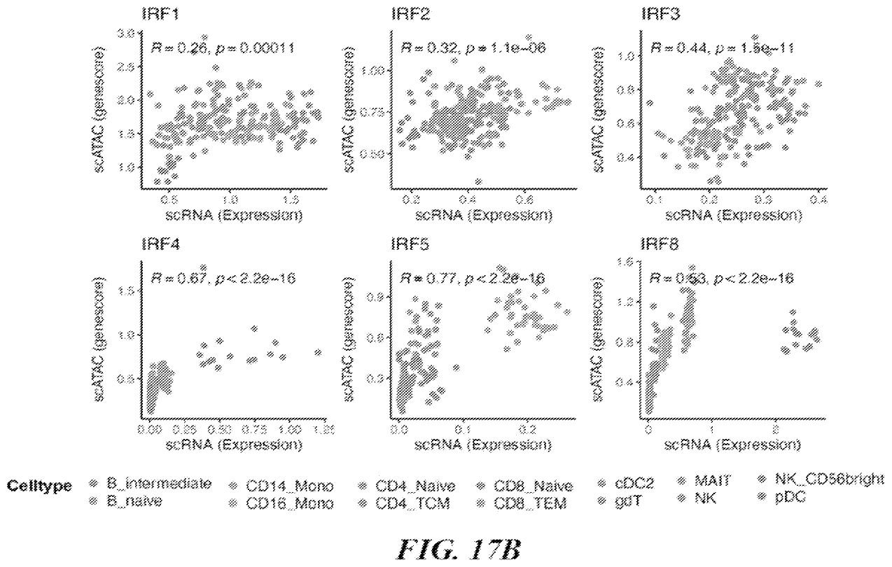

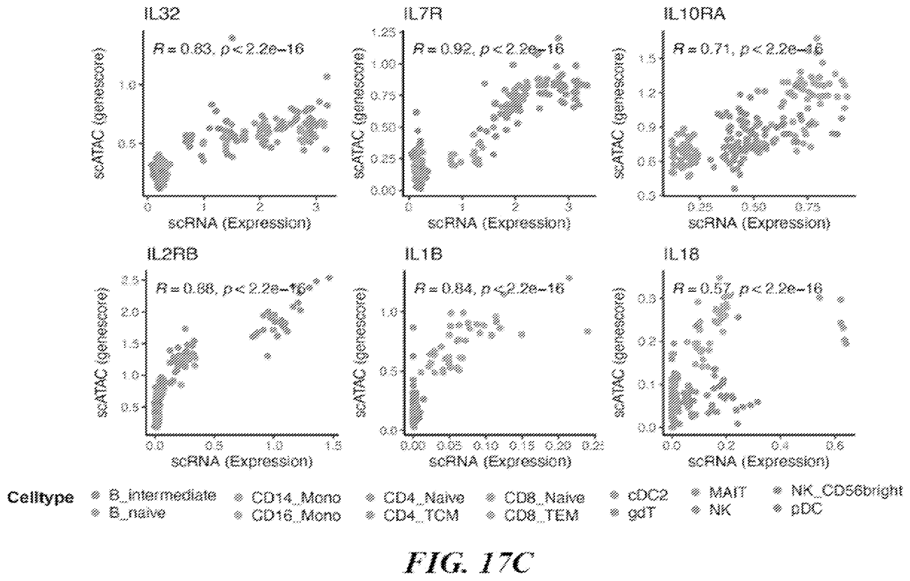

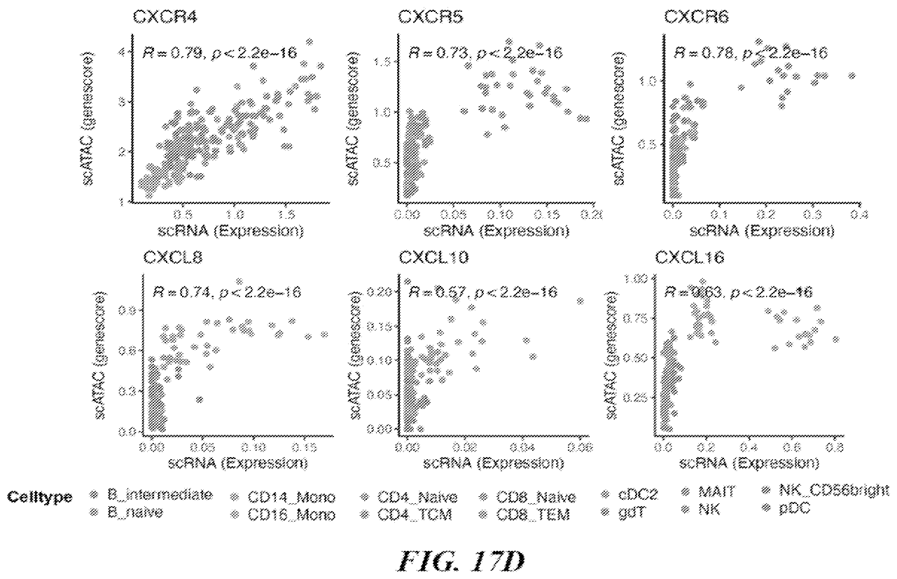

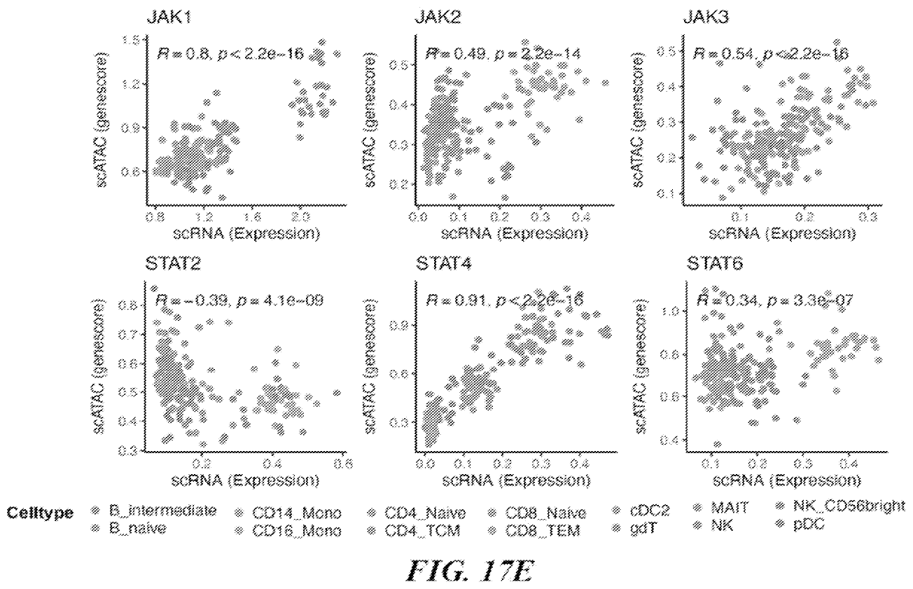

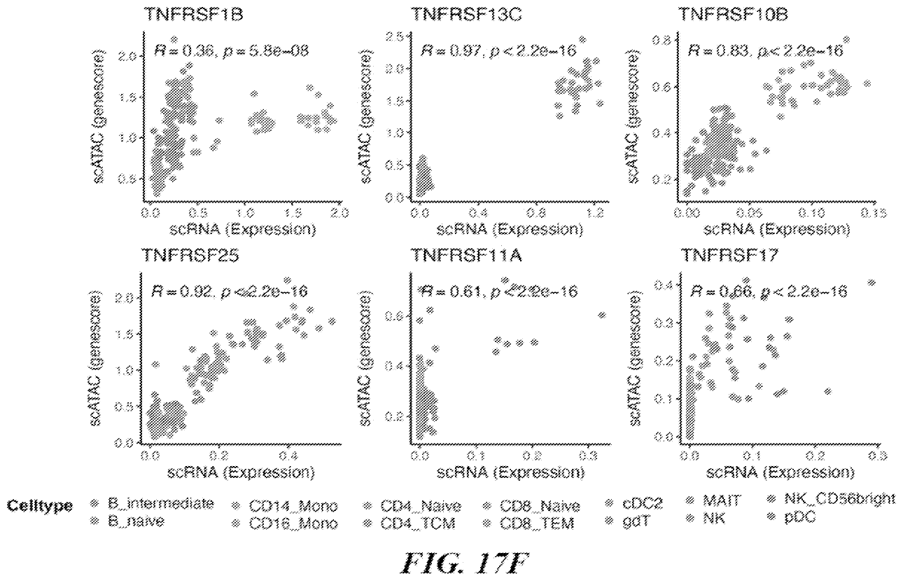

PALMO implements circos plots to display stability patterns from multiple single-cell data modalities together. We displayed the stability pattern of gene expression and gene score of six protein families that are essential for immunity in FIGS. 6A-6F, including: human leukocyte antigens (HLAs, FIG. 6A), interferon regulatory factors (IRFs, FIG. 6B), interleukins (ILs, FIG. 6C), chemokine (C-X-C motif) receptor/ligand (CXCR/L) family (FIG. 6D), Janus kinases (JAKs) and signal transducer and activator of transcription proteins (STATs, FIG. 6E), and tumor necrosis factor receptor superfamily (TNFRSF, FIG. 6F). All these protein families showed diverse stability patterns among members and across cell types, with HLAs and ILs having the most striking contrasts. The rich variety in such stability patterns suggests that different members of the protein superfamilies may play different roles in individual cell types. We noticed that gene expression and gene score generally did not exhibit the same stability patterns despite the rather strong correlations between them (FIGS. 17A-17F). It turns out that strong correlations were mainly driven by difference between cell types rather than difference between samples, likely reflecting the complexity of gene regulation as mentioned before.

### Example 9: Application of TCA to Reveal Heterogenous Immune Responses Among COVID-19 Patients

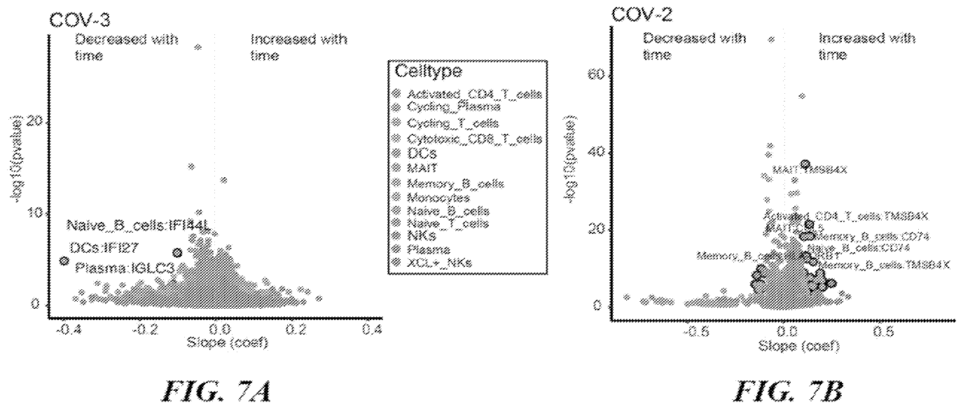

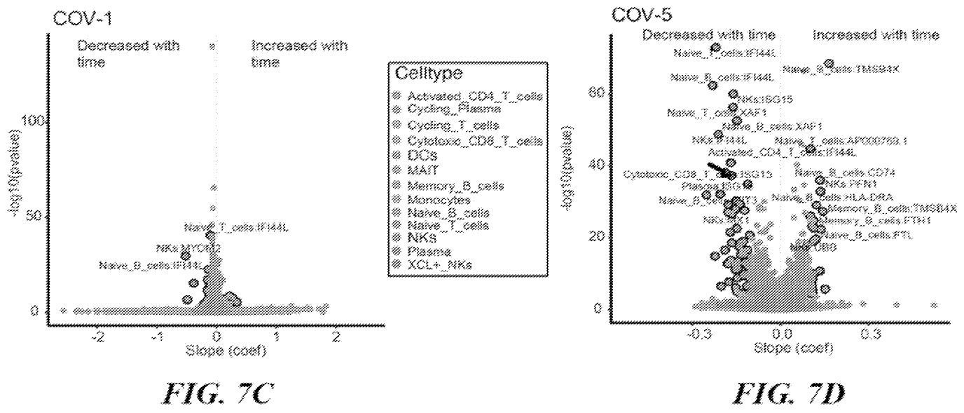

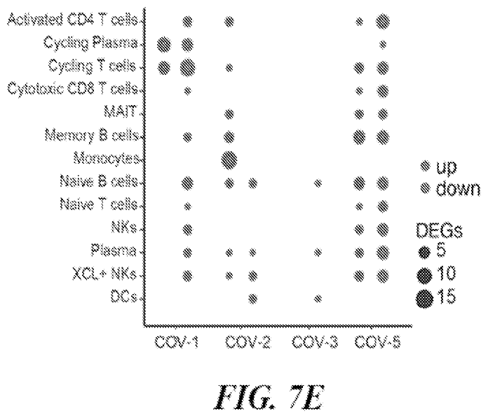

We applied TCA to analyze longitudinal scRNA-seq data of four COVID-19 patients, each having data of at least three timepoints, in a previous study, and identified significantly up- or down-regulated genes over time (adjusted p<0.05 and slope magnitude >0.1, FIGS. 7A-7D, Table 7A) and the corresponding pathways (Table 7B). We observed rather heterogeneous immune responses by these patients during recovery (FIG. 7E), which was not presented in the original study.

Patient COV-3 had barely any significant genes except that IFI27 decreased in DCs, IFI44L decreased in naive B cells, and IGLC3 decreased in plasma cells, suggesting possible dampening of immune modulation.

The significant genes of patient COV-2 included eighteen upregulated genes in monocytes, four genes each in memory B cells and naive B cells, and twelve genes split among other six cell types. Gene enrichment analysis on the eighteen upregulated genes in monocytes revealed only one significant pathway: myeloid leukocyte mediated immunity (adjusted p=0.044).

The significant genes of COV-1 included eleven upregulated and six downregulated genes in cycling plasma cells, seven upregulated and sixteen downregulated genes in cycling T cells, six downregulated genes in naive B cells, and fifteen genes split among other seven cell types. The significant genes in cycling plasma cells are significantly enriched in five pathways, including regulation of humoral immune response (adjusted p=3.92×10⁻³), Fc receptor mediated stimulatory signaling pathway (adjusted p=3.92×10⁻³), and immunoglobulin production (adjusted p=0.011), indicating a predominant role of humoral immunity in the recovery of the patient.

Patient COV-5 had significant genes in almost all cell types except for DCs and monocytes, including eight upregulated and eight downregulated genes in memory B cells, six upregulated and six downregulated genes in naive B cells, one upregulated and ten downregulated genes in activated CD4+ T cells, two upregulated and eight downregulated genes in plasma cells, and 43 genes split among other seven cell types. Seven (58%) of the twelve significant genes in naive B cells were also significant in memory B cells and in the same direction of change, suggesting common responses by the two cell types. The significant genes in memory B cells are enriched in interferon gamma (adjusted p=3.28×10⁻⁶) and alpha (adjusted p=4.86×10⁻⁵) response, antigen processing and presentation (adjusted p=0.036), and antigen processing and presentation of peptide or polysaccharide antigen via MHC class II (adjusted p=0.044). The significant genes in naive B cells are enriched in interferon-alpha (adjusted p=1.96×10⁻⁵) and gamma (adjusted p=1.96×10⁻⁵) response. The significant genes in plasma cells were enriched in innate and humoral immune responses (p=3.46×10⁻⁴ and p=5.79×10⁻⁴, respectively) although both with an adjusted p=0.084. These results align to the patient's disease severity and advanced age.

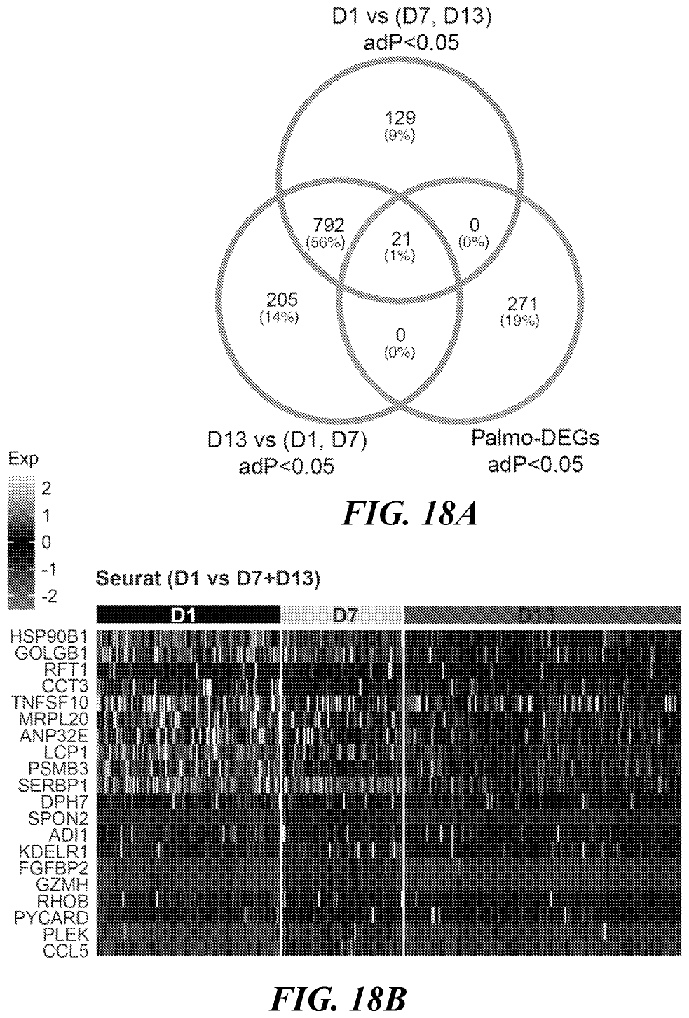

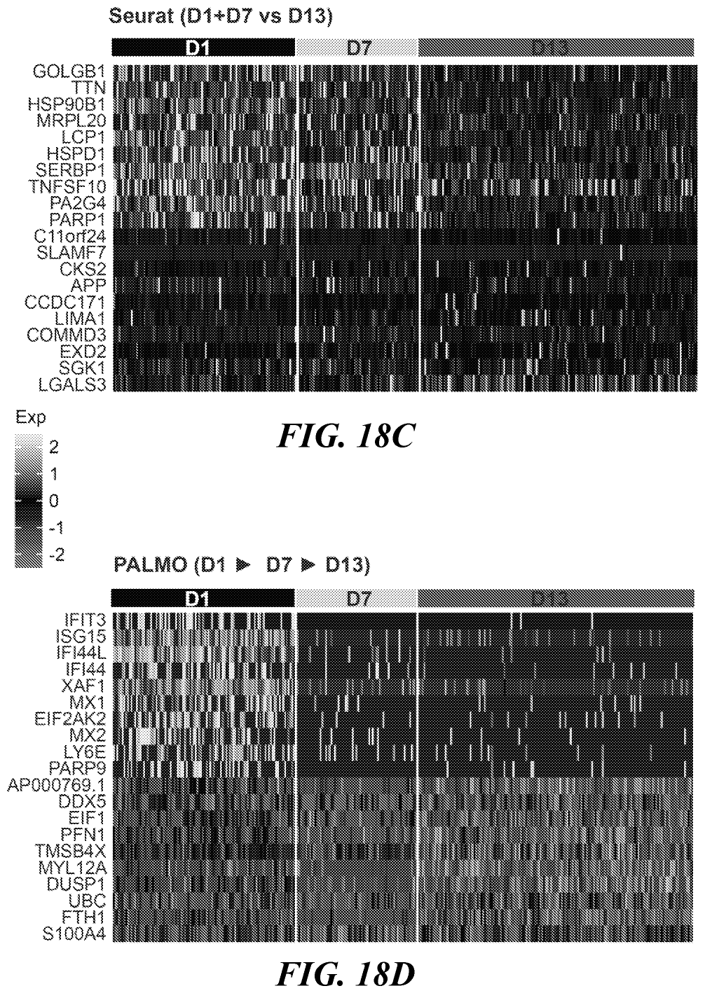

For comparison, we also used Seurat to analyze patient COV-5 data of activated CD4+ T cells. To satisfy Seurat's requirement of selecting two contrast groups, we did the analysis in two iterations (i.e., day 1 (D1) versus D7+D13 and D1+D7 versus D13), and obtained 942 and 1,018 DEGs (adjusted p<0.05), respectively, with an overlap of 813 DEGs (FIG. 18A). TCA identified 921 significantly up- or down-regulated genes (adjusted p<0.05), only 21 of which overlapped with both Seurat results. The genes obtained from TCA or Seurat were quite different. We collected top ten up- and top ten down-regulated genes from all three approaches and plotted the corresponding gene expression in heatmaps (FIGS. 18B-180). TCA results showed better dynamic changes over time than Seurat results.

Taken together, the data demonstrated in the examples above show that the five modules in PALMO analyze longitudinal omics data from multiple perspectives as continuous data. VDA provides a global view on the sources of variance within the whole dataset. TCA studies the time series of individual participants. CVP and SPECT first examine data of individual participants separately and then summarize the observations across different participants. All these four methods focus on individual features. ODA is the only method to provide a sample-level analysis. Which module(s) to use on a specific dataset depends on the research question of interest.

We observed that a small set of STATIC genes, 220 for PBMC and 304 for mouse brain tissues, distinguished cell types well and captured some biological differences. The PBMC STATIC genes showed better correlation between gene expression in scRNA-seq data and gene score in scATAC-seq data than HVGs. It would be interesting to see whether these observations can be extended to scRNA-seq data of other sample types.

Plasma proteins are often targeted as disease biomarkers, thus understanding their temporal stability is of particular interest. Conceptually, highly variable proteins are poor biomarker candidates since their values likely have very high sampling variations. The rather moderate CV values of the most variable proteins in our study suggest sampling variations are not a big concern on these proteins. The small CV values of the most stable proteins, on the other hand, indicate they do not change much under normal, healthy conditions. So, if they ever change under some disease conditions, they should be closely explored as potential biomarkers.

We condensed single-cell data into pseudo-bulk data in VDA, SPECT and ODA. Recent literature revealed that many single-cell methods fail to properly account for variations in cross-sectional scRNA-seq data and generate many false DEGs as a result. In comparison, pseudo-bulk approaches mostly generate reliable results although they may be underpowered. Longitudinal single-cell omics data is even more complicated than cross-sectional scRNA-seq data and may require new statistical methods to properly handle its many types of variations. Furthermore, memory and CPU requirements for using GLMMs to analyze longitudinal single-cell omics data at single-cell level may be challenging even for cloud-based computing. We adopted the pseudo-bulk approach in VDA, SPECT and ODA as a practical compromise. In TCA we bypassed some of the complications by analyzing data of individual cell types and of individual participants separately.

The lack of a well-accepted software package for longitudinal omics data makes it difficult to benchmark PALMO performance. We compared PALMO with variancePartition, tcR, and Seurat, which is summarized in Table 1C. VDA can handle missing data but variancePartition cannot, which is an advantage of VDA since missing values in longitudinal omics data are almost inevitable. The two tools generated almost identical results on two tested datasets after removing missing values. PALMO was not developed specifically for TCR data. When we applied VDA to the TCR data of SSc donors, we obtained results that are potentially interesting but not reported in the original study using tcR. We believe PALMO complements TCR specific tools (such as tcR) on TCR data. Seurat requires users to select two contrast groups in DEG analysis and thus is not appropriate for analyzing longitudinal data of more than two timepoints. Nevertheless, when we applied both TCA and Seurat to the longitudinal scRNA-seq data of activated CD4+ T cells of a COVID-19 patient, the two methods generated rather different results on up- or down-regulated genes. Heatmaps of the corresponding top genes revealed that TCA results showed better dynamic changes over time than Seurat results. Accordingly, PALMO can be used to analyze longitudinal bulk and single-cell omics data generated on diverse technical platforms and/or of diverse sample types, including, but not limited to, clinical lab test results, cell type composition, gene expression, protein abundance, bulk or single-cell omics data, and TCR sequencing data.

### Example 10: Application of the STATIC 220 Genes to Identify Donors Potentially Having Monoclonal B Cell Lymphocytosis (MBL)

Exploratory analysis of the STATIC 220 genes revealed several interesting features of these genes. First, we noticed the genes had distinct patterns across cell types and hypothesized that some of these genes were potentially good markers for cell types. To test our hypothesis, we projected the cells in scRNA data on a two-dimensional UMAP, using the 220 STATIC genes as input features, and kept fifteen principal components (PCs). We further generated UMAPs using the same 220 STATIC genes (with fifteen PCs) on four independent, longitudinal scRNA-seq datasets. In all five cases, the 220 STATIC genes separated major cell types and most of their subtypes very well, supporting our hypothesis for using these 220 STATIC genes in cell labeling.

We observed some participants having abnormal B cells in our studies. Further analysis based on flow cytometry suggested that these participants potentially had a precancerous condition called monoclonal B cell lymphocytosis (MBL), which increases the risk of developing a blood cancer called chronic lymphocytic leukemia (CLL). MBL refers to a monoclonal population of B lymphocytes <5,000 cells/microL (<5×10⁹/L) in peripheral blood for 23 months, without other features of a B cell lymphoproliferative disorder. MBL shares the immunophenotypic characteristics of CLL and accounts for three-quarters of MBL cases.

To further demonstrate the utility of STATIC 220, we performed flow cytometry and single cell RNA-seq analysis on PBMCs samples of 16 participants, including four participants likely having MBL and 12 healthy controls. We showed that the STATIC 220 genes were able to separate the abnormal B cell populations well.

The following methods were used in this example:

Healthy donors: We enrolled 16 clinically healthy donors with age between 31 to 77 years and includes 9 males and 7 females. Blood samples were obtained from Benaroya Research Institute (BRI) and Colorado University (CU) through protocols approved by the respective institutional review board. The cohort demographics are described in the Table 8.

scRNA-sea data analysis: scRNA-seq individual HDF5 files were loaded into the R statistical programming language (version 3.6.0) using Bioconductor (version 3.1.0) and the Seurat package (version 4.0). We calculated read depth, mitochondrial percentage, and number of UMIs per sample. Cells were filtered with nFeature_RNA>200 and percent.mt<10. The merged data structure was normalized (using NormalizeData and FindVariableFeatures functions) and then saved as an RDS for further analysis. The top 3000 variable genes were used for PCA and UMAP based dimension reduction maps using 30 principal components (PCs). We checked for possible batch effects using the bridging controls but did not observe any obvious batch effects. Label transfer was performed using previously published procedures (Stuart et al., Cell (2019) 177:1888-1902) and with the Seurat reference dataset. The UMAP was visualized using Seurat V4.

B cell sub setting: We used subset function from Seurat V4 to subset the B cells from single cell data. The level-2 cell types consisting of pre-B cell and B cell progenitor were considered as B cells. In total, we obtained 8806 B cells from 16 participants and used for downstream analysis.

Density estimation: The density estimation is based on the 2-dimensional UMAP coordinations. We use the kde2d function in the R MASS package to estimate the 2D kernel density. To ensure the equal representation of density for each sample, the number of cells on each sample within a density estimation are equal to the mean number of cells in all samples by random selection with replacement.

Enrichment analysis: Overrepresentation enrichment analysis (ORA) was performed using R package clusterProfiler v3.16. The enrichment geneset was gene ontology biological processes under “immune response” (G00006955) category. The geneset was obtained from MsigDB v7.2. The geneset consists of 90 immune-specific pathways and 2,800 genes. Enrichment terms with p<0.05 were considered as significant.

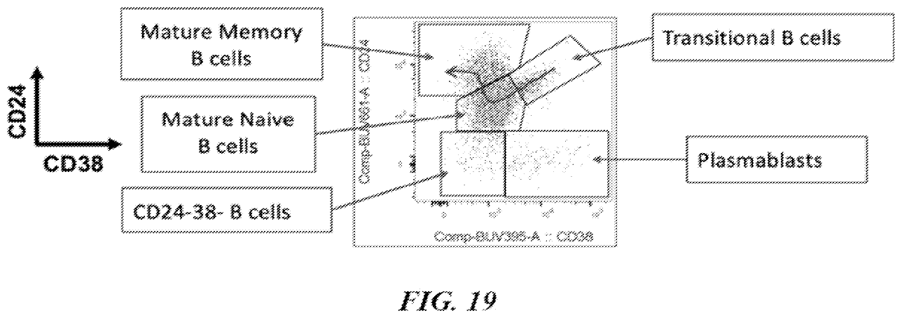

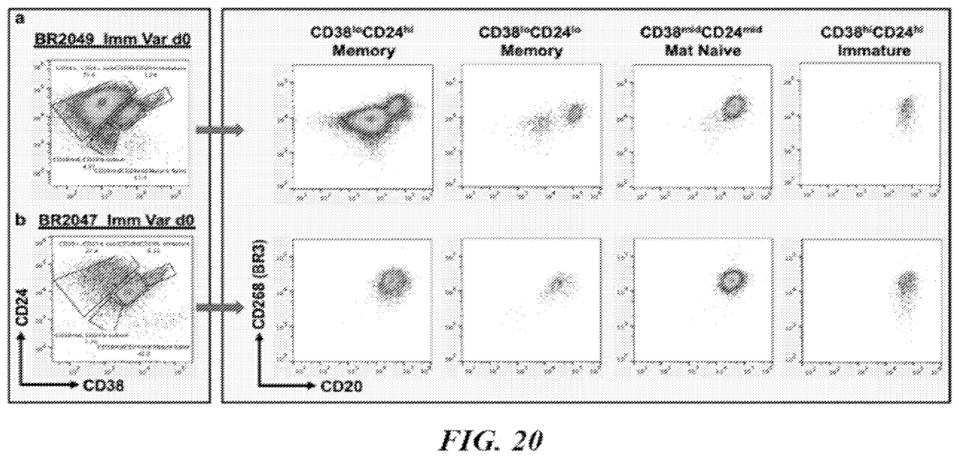

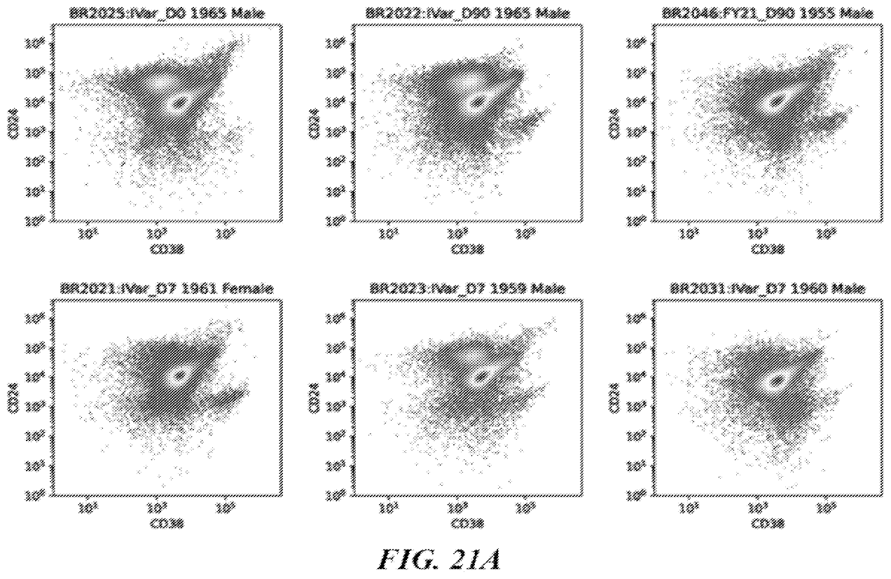

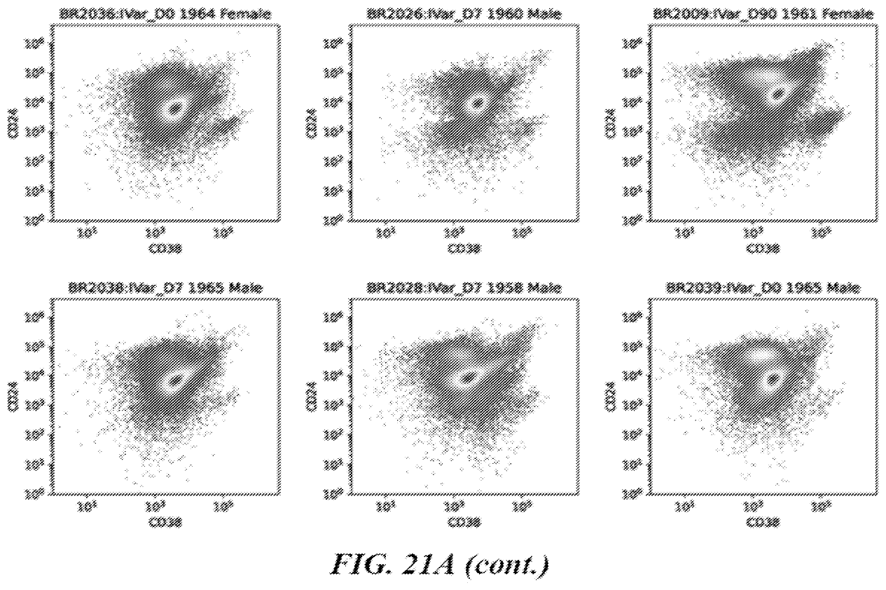

We collected blood samples from 16 participants, whose demographic details are summarized in Table 8. The flow cytometry data from these 16 donors were obtained. The B cell population from flow data was analyzed using CD38 and CD24 markers in a flow gating strategy as shown in FIG. 19. MBL is characterized by a high clonal expansion of cells with mature memory B cell like characteristics, which can be identified as CD₃₈^{lo}CD₂₄^{hi} B cells (FIG. 20). Other flow characteristics observed in abnormal memory B cell population are: CD20low, CD268low, CD38low, CD40mid-low, CD45lower, CD85jNeg, CD86Neg-Mid, IgMNeg, IgDNeg-Mid, IgANeg, and IgGNeg. Among 16 participants, we observed 4 participants with abnormal mature memory B cells on the flow panel (FIGS. 21A-21B). These results strongly suggested the 4 participants likely had MBL.

To show the utility of the STATIC 220 genes in separating the abnormal B cell population using scRNA-seq data, we performed single cell RNA-seq on the 16 participant PBMC samples. In total we obtained 80,000 cells from the 16 participants and measured expression for 33,538 features (gene expression). We further extended the STATIC 220 gene list to 500 gene list consisting of the 220 STATIC genes and 280 hand-picked immune-related genes based on literature (Table 9). We aimed to demonstrate the utility of both the STATIC 220 genes and the 500 gene list.

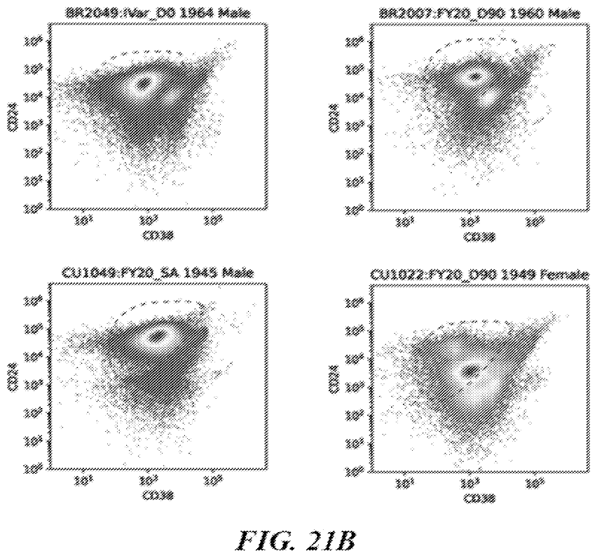

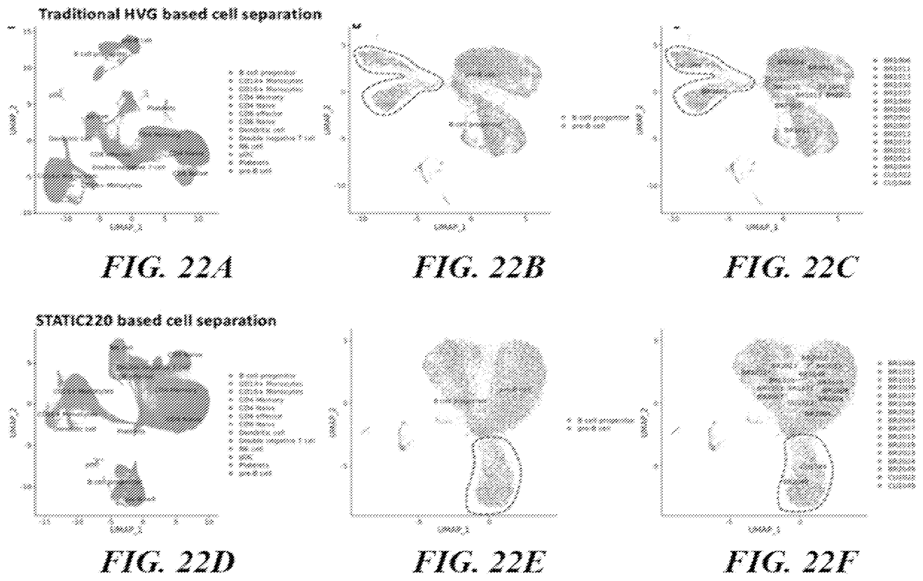

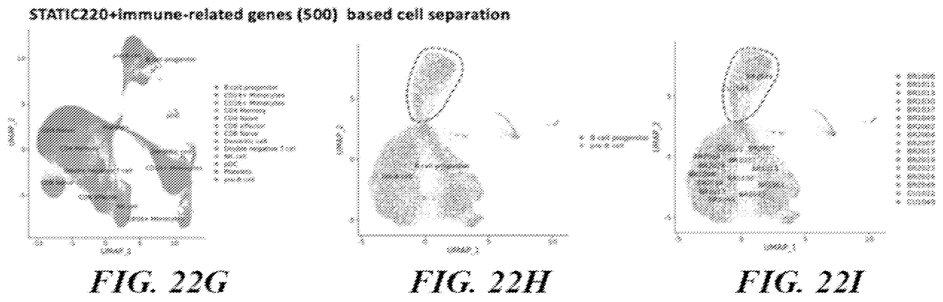

scRNA-seq data was log-normalized and highly variable genes were obtained. The 3,000 HVGs were used to perform PCA on high-dimensional scRNA data and project the data to low-dimensional space (PCs=50). The data was visualized in UMAP using Seurat (FIG. 22A). We also compared the results from the 3000 HVGs, the 220 STATIC genes, and the 500 gene list (FIGS. 22A-22I). The dot color represents identified cell types based on Seurat V2. These results confirmed our previous findings that the STATIC 220 genes and the 500 gene list were able to separate cell types well.

B cells were isolated from single cell data, clustered, and projected on UMAP (FIGS. 22B, 22C, 22E, 22F, 22H, 22I). Using the 3000 HVGs, the B cell clusters included two clusters of normal B cells (pre-B cell, B cell progenitor) and two clusters of abnormal B cells as highlighted in dashed lines (FIGS. 22B-22C). Using the STATIC 220 genes or the 500 gene list, the two clusters of normal B cells remained while the two clusters of abnormal B cells were merged into a single cluster (FIGS. 22E, 22F, 22H, 22I). Thus, the STATIC 220 genes and the 500 gene list can clearly separate abnormal B cells from normal B cells, demonstrating their utility on clinical usage. In addition, by merging the two clusters of abnormal B cells into one, they simplify the interpretation of the results.

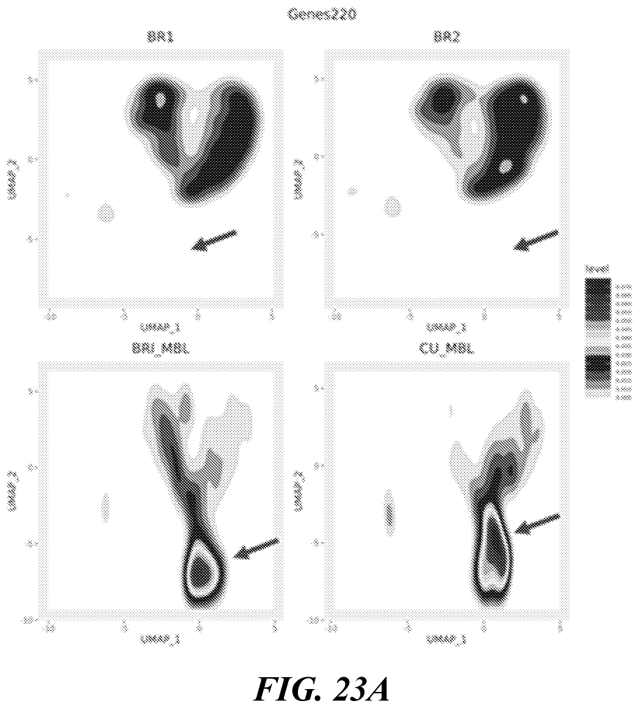

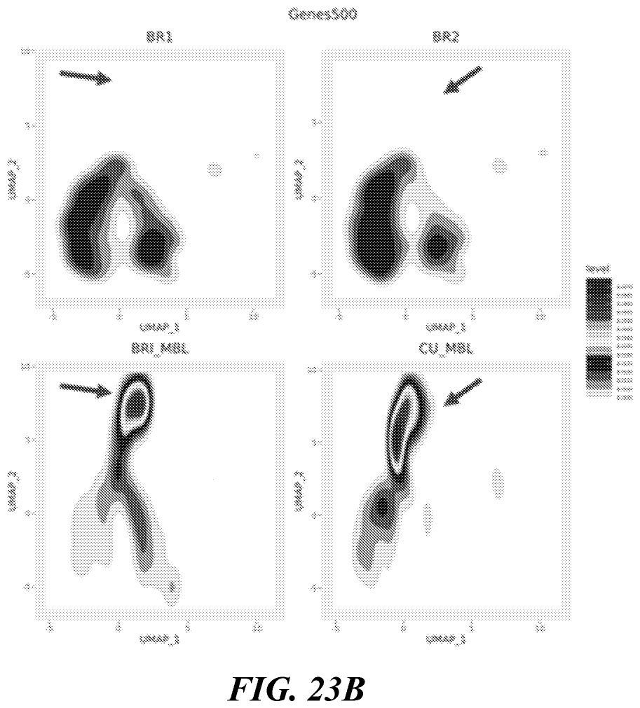

To further illustrate their utility, we generated UMAP density plots on B cells using the STATIC 220 genes (FIG. 23A) or the 500 gene list (FIG. 23B), comparing B cells of two healthy participants with those of likely MBL participants. The strong contrast between the two types of participants indicates that the STATIC 220 genes and the 500 gene list can be powerful in the diagnosis of some diseases.

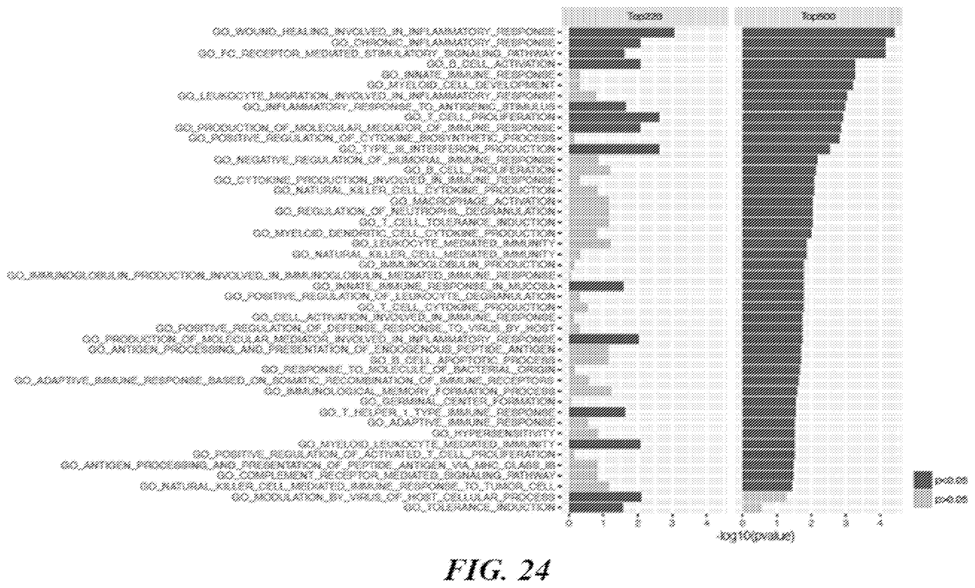

We further enriched the STATIC 220 genes for pathway enrichment analysis. The pathway enrichment by over representation analysis shows that the STATIC 220 genes are majorly associated with inflammatory related and adaptive response biological processes (FIG. 24). For example, the top pathways include wound healing involved in inflammatory response, chronic inflammatory response, type II interferon response, and TH1 immune response (p<0.05).

We expanded the STATIC 220 genes to include more immune-related genes which resulted in the 500 gene list. The 500 genes are highly interactive and enriched in both innate and adaptive immune response and many genes mutually interact with each other.

To show application of the STATIC 220 genes in label transfer, we downloaded the Seurat V4 PBMC reference. The reference consists of 20957 features across 161764 cells. We obtained scRNA-seq data from in-house database consisting of 97 participants from different cohorts (young adults, n=20, BR1 cohort; older adults, n=22, BR2 cohort; RA disease, n=22, Colorado cohort; multiple myeloma, n=15, Fred-Hutch cohort; melanoma, n=18, UPenn cohort). With big scRNA-seq data from 97 samples, 2,500 cells per sample were selected randomly which resulted in a final dataset with 194,000 cells and 97 samples.

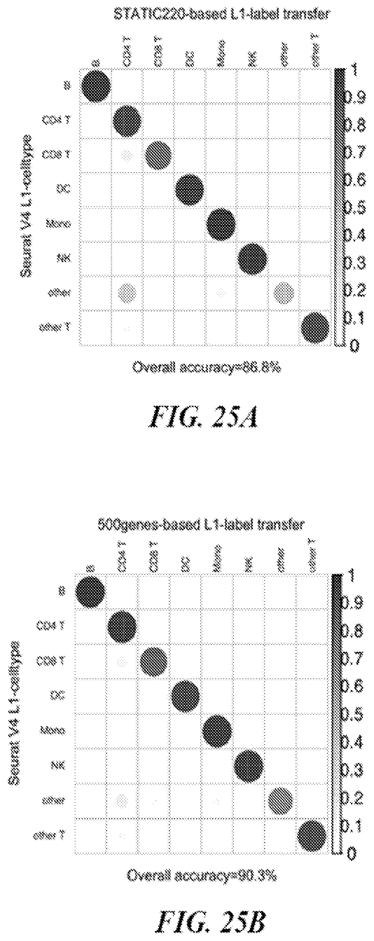

We performed the label transfer by Seurat V4 on the final scRNA matrix using Seurat reference. Then we performed label transfer on the final scRNA matrix focusing on only the STATIC 220 genes or the 500 genes. The label transfer was then compared, and proportion matrix was drawn between Seurat V4 based L1 transfer and STATIC 220-based L1-label transfer (FIG. 25A) and Seurat V4 based L1 transfer and 500-based L1-label transfer (FIG. 25B). The overall accuracy was calculated as a sum of diagonal proportions divided by the number of cell types under consideration. On average, we observed 86.8% accuracy from the STATIC 220 genes only while 90.3% accuracy from the 500 genes. Overall, the high accuracy from the STATIC 220 genes-based label transfer certifies its utility on label transfer, compared to the conventional method using more than 20,000 genes.

Accordingly, the analysis of abnormal B cell populations shows that the STATIC 220 genes and the 500 genes can separate the abnormal B cell clusters without being confounded by donor specificity. This is important because researchers and clinicians are most interested in identifying disease specific outliers or abnormal expression profiles in participants rather than donor-specific differences. Enrichment analysis on the STATIC 220 genes suggests that they are mostly associated with inflammatory biological processes. The cell type label transfer by the STATIC 220 genes showed ˜87% accuracy at level 1, justifying their usage for labeling immune cell types.

### Example 11: Application of the STATIC 220 Genes to Stratify Cancer Patients of Multiple Myeloma

Multiple myeloma (MM) is a type of plasma cell cancer that arises from bone marrow. In this example, we will demonstrate how the STATIC 220 genes can help differentiate the MM samples from samples of other conditions. In the MM cohort, we have pre-treatment samples (FH1_PreTreatment) and samples after induction therapy (FH1_PostInduction). In the control group, we have samples of healthy young adults (BR1), healthy older adults (BR2), participants with a high risk to rheumatoid arthritis (CU), participants having clinical rheumatoid arthritis (CU_Clinicial_RA), and participants having melanoma (UP2).

The follows methods were used in this example:

Sample selections: From each cohort, we selected about 14-20 scRNA samples based on availability in our database. For each selected sample, we randomly selected 5,000 cells. We collected the expression of the STATIC 220 genes from the entire gene expression matrix. We randomly split the samples into training and testing groups. The training group was used to assert whether there is a difference between MM patients and others. The testing group was used to validate that the difference was not due to donor or sample variations.

Classification of clustering: On the training dataset, we first performed log-normalization on all cells. Then, we did cell-wised scaling and trained a PCA model on the data. We performed UMAP analysis by using the top 10 PCs and setting parameter k of nearest neighbors to 30. Finally, we used the Leiden algorithm with a resolution equal to 0.25 to find the clustering assignment.

We used the KNN algorithm to develop a KNN model for predicting cluster assignment on any new dataset. To find the parameters of the KNN model and evaluate its accuracy, we performed 5-fold cross validation on the training dataset. Afterwards we trained a KNN model based on the whole training dataset and tested its performance on the testing dataset.

On the testing data set, we first performed log-normalization and cell-wised scaling. To find the corresponding cluster, we used the trained PCA model from the training dataset to transform normalized and scaled testing dataset into the same PCA space. By using the top 10 PCs of the testing dataset as input, we used the trained KNN model to predict corresponding clustering assignments.

Modeling and Projection: For each cluster in the training dataset, we performed feature scaling first. After feature scaling, we trained a new PCA model and UMAP model for each cluster. UMAP models were trained based on the top 10 PCs. For the testing dataset, we used the same PCA model and UMAP model to perform the transformation of corresponding clusters.

Centered log ratio (CLR) transformation: We use the R package “composition” to do CLR transformation. CLR transformation is performed based on cluster's frequency per sample.

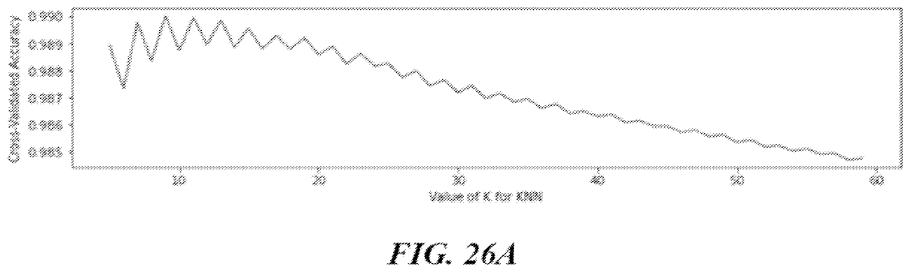

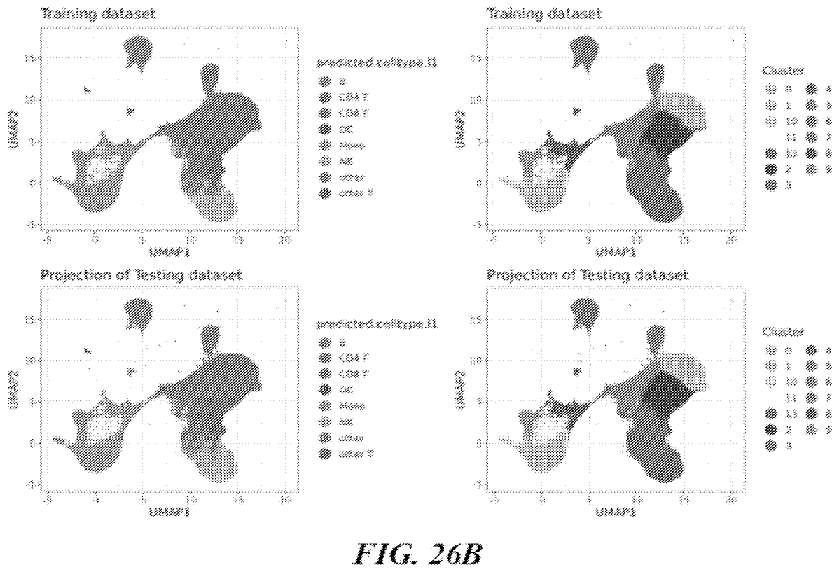

The overall accuracy of the KNN model was around 0.98-0.99 for the value of K tested on KNN model (FIG. 26A). We chose K equals 30 for all the final models because the accuracy difference between different K was very small, and because the UMAP of the training dataset also used K=30 for calculating neighborhood graphs. The projection of the testing dataset showed the same structure with the training dataset, and the predicted cluster of testing dataset was on the same location of testing dataset (FIG. 26B). This shows that the KNN model can successfully predict the clustering assignment of the testing dataset.

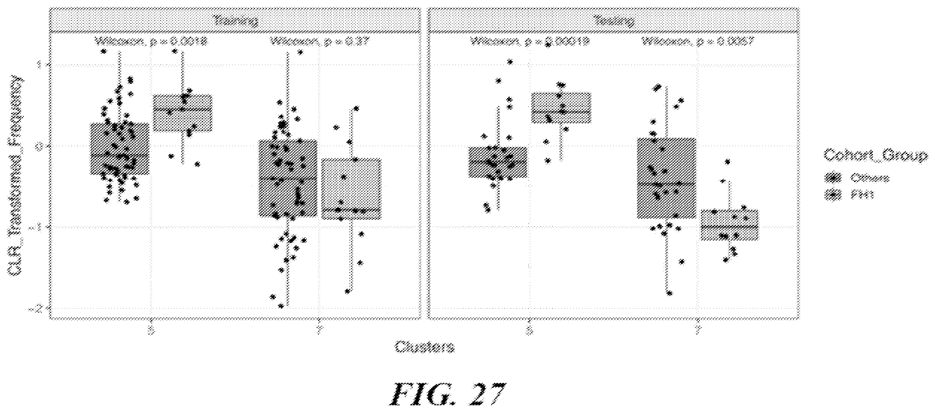

To demonstrate how to use the STATIC 220 genes to diagnose multiple myeloma (MM), we calculated cell frequency in the 10 KNN clusters for each sample. We compared CLR transformed cell frequencies between samples in the FH1 group (MM) with samples in other cohorts (FIG. 27). Cluster 5 showed a significant difference in both the training (p=0.0018) and the test (p=0.00019) datasets. Cluster 7 showed a significant difference in the test dataset (p=0.0057) and the same trend, though a not significant difference, in the training dataset (p=0.37). The results confirmed that the STATIC 220 genes can be used in MM diagnosis.

We showed that the STATIC 220 genes can help us to separate the MM cohort with other health and disease cohorts. We successfully predicted the cluster assignment of the testing dataset. The overall accuracy was very high. We also identified two clusters whose cell frequencies showed clear differences between the MM cohort and other cohorts, which can be utilized in MM diagnosis.

### Example 12: Application of the STATIC 220 Genes in Spatial Transcriptomics

Spatial transcriptomics is a newly emerging and rapidly evolving molecular method that enables the genome-wide profiling of gene expression within the nascent tissue context with single cell resolution, thereby facilitating an integrative understanding of tissue identity in both health and disease. To demonstrate the usefulness of the STATIC 220 genes as defining features for cell type identification in spatial transcriptomics, we probed the expression patterns of 183 out of 220 genes in the STATIC 220 panel in a tonsil sample using the Vizgen MERSCOPE platform and performed dimension reduction and cell type clustering on the resulting dataset.

The following methods were used in this example:

Spatial transcriptomics data generation and postprocessing: The spatial transcriptomics of a tonsil sample was generated on the Vizgen MERSCOPE platform according to the manufacturer's specification. A custom designed gene panel was used as probes for gene expression query. 183 out of a total of 443 genes in the gene panel are from STATIC 220. The number of transcripts per gene per cell was determined by first defining cell locations by performing cell segmentation of the microscopic images of the tissue using cellpose and subsequently counting the number of transcripts per gene within the geometric space of the cells defined by cellpose. The output, named cell-by-gene matrix, was used for downstream dimension reduction and cell type clustering.

UMAP clustering: The cell-by-gene matrix for tonsil output by the postprocessing step in which the rows were cells and the columns were genes, was standardized row-wise according to the equation Xi=(Xi−Xmean)/std(Xi) where Xi was row i in the cell-by-gene matrix, Xmean was the row mean and std(Xi) was the row standard deviation. The standardized array was decomposed into 40 principal components (PCs) using principal component analysis (PCA) and then subsequently projected into the UMAP space. Leiden clustering of cells was performed on the 40 PCs post PCA until convergence.

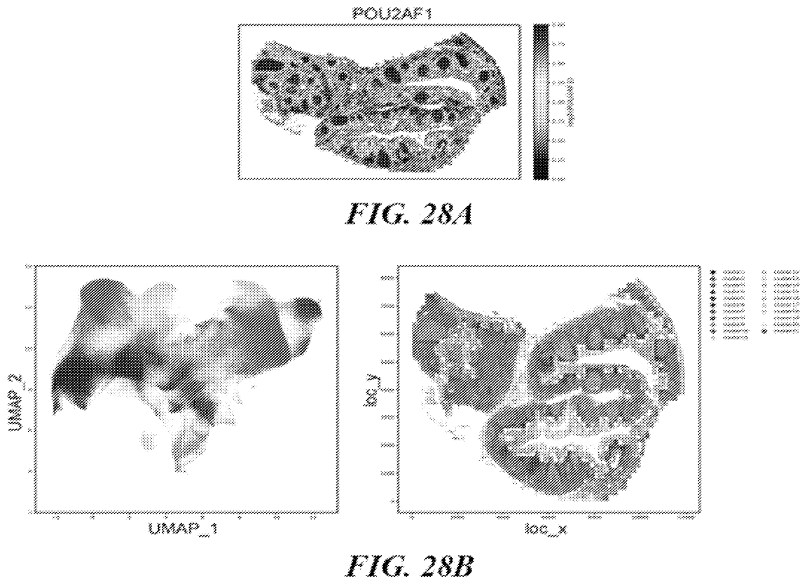

To determine the spatial distribution of the STATIC 220 genes in tissues, thereby assessing their value in defining cell types in immune tissue space, we profiled the expression patterns of 183 of the STATIC 220 genes in a human tonsil sample. We find that many genes show distinct domain specific expressions. For instance, POU2AF1 shows great enrichment in almost exclusively structures known as germinal centers in the tonsil (FIG. 28A). Further, by performing UMAP dimension reduction and subsequently Leiden clustering the cells based solely on the expression of the 183 genes, we show that the features defined by 183 genes are sufficient to produce well separated cell clusters in the UMAP space, as well as tissue space, indicating distinct cell types (FIG. 28B). Overall, we conclude that the STATIC 220 genes are information rich features in spatial transcriptomics and are sufficient for differentiating cell types in immune tissues.

### Example 13: Fixed RNAseq Assay on the STATIC 220 Genes

10× Genomics' new Chromium FRP kit is going to enable near whole-transcriptome level gene expression profiling while greatly scaling the number of cells that we can capture in a single experiment, as well as reducing cost. This new assay will probably become the workhorse assay that phases out the standard V3.1 3′ assay. With this in mind, we want to show the power of detection of the STATIC 220 gene panel in the FRP panel is as strong as V3.1 3′ assay.

We have two main experiments here that we can use. The first one was to test multiple conditions on whole blood preservation and compare between FRP & 3′ chemistries. We can use this experiment to answer the question of power of detection by comparing the level of expression of the STATIC 220 genes in FRP versus V3.1 in the same samples/conditions. The second one will be the experiment that we can start to dive into DEGs and pathways to see if we can still pick up similar differences in expression by comparing changes detected by the 18,000 gene panel and the STATIC 220 gene panel.

The following methods were used in this example:

Detection of the STATIC 220 genes in fixed RNA profiling chemistry: To address our ability to detect the STATIC 220 genes in the Fixed RNA Profiling assay, a single donor had PMBCs isolated from whole blood via Ficoll, and stored frozen in DMSO following our current pipeline procedures. Upon thawing, the sample was run on both 10× 3′ and 10× FRP chemistries following 10×'s protocols. Sequencing was targeted for similar depths on each chemistry and the FASTMs were passed through CellRanger 7.0. To investigate how the STATIC 220 genes compare between chemistries, we calculated the average number of UMIs per cell for each gene in both the STATIC 220 panel and whole-transcriptome.

Performance of the STATIC 220 genes with label transfer: Seurat's TransferData method was applied using a reference dataset to transfer cell type labels to the same sample using either the full 18,000 gene panel or the subset STATIC 220 gene set.

DEG analysis: PBMCs were subjected to either a stim condition (PMA/lonomycin/ODN) or control condition (PBS) for 3 hours. The samples were then processed on 10×'s FRP assay and sequencing results were aligned in CellRanger. Standard QC cutoffs were applied, and label transfer was performed using Seurat's whole PBMC reference. Seurat's FindMarkers was then run to identify differentially expressed genes (DEGs) by comparing major cell types between the stim and control condition in both the full ˜18,000 and the STATIC 220 gene panel.

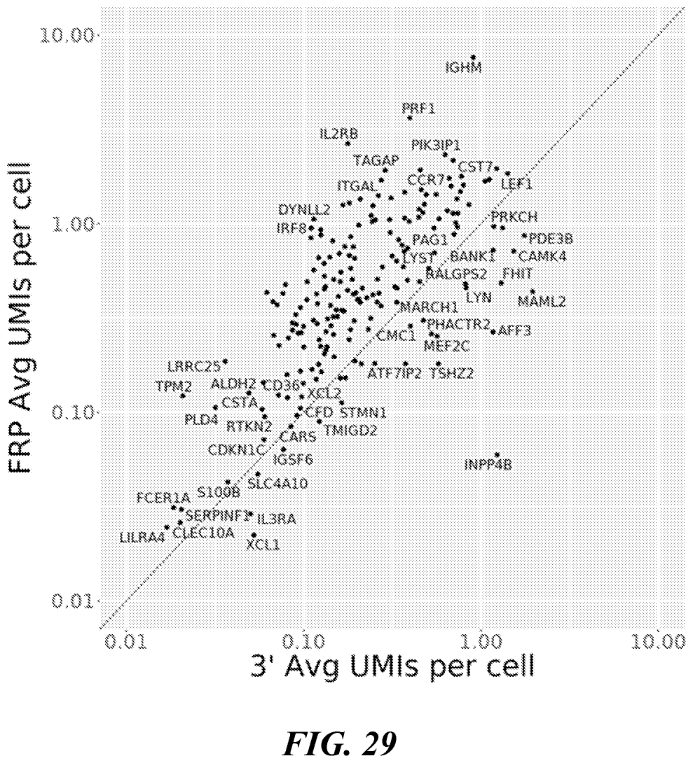

In the 10× FRP chemistry, 200 out of the 220 STATIC genes were included in the FRP probe set. The genes not included are LINC00861, AC243960.1, IL6ST, MHENCR, CD8B, LINC02446, A1BG, CYTOR, TRG-AS1, LINC01871, LINC00623, HLA-DQA1, LINC00926, HLA-DMA, IGLC2, HLA-DMB, LINC01857, FCN1, AC020656.1, and SMIM25. The FRP chemistry also had a higher sensitivity in 86% of these genes (FIG. 29).

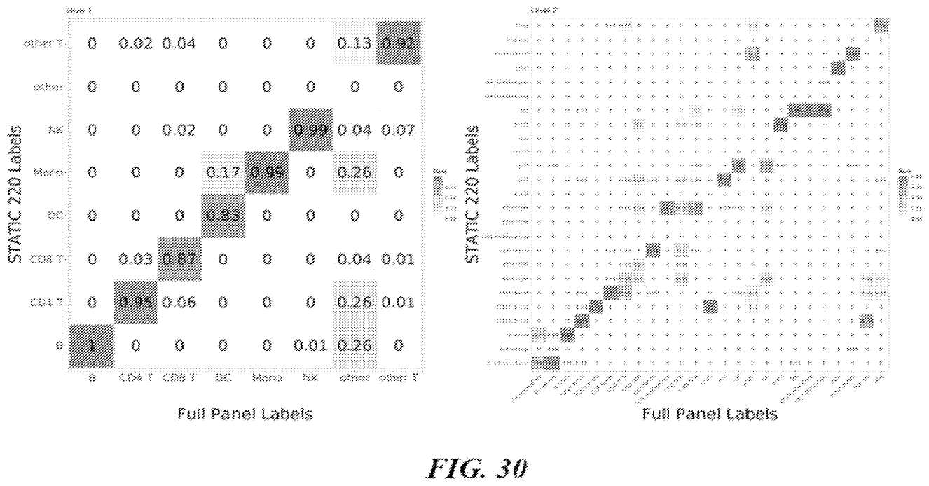

We wanted to address the ability of label transfer to still function if a user were to only use the STATIC 220 gene panel with the FRP assay. At the broader “Level 1,” there was a 94.8% correlation of cell type labels between the full panel and the STATIC 220 panel. The largest cell type populations performed the best while the rarer populations struggled to align. For the more specific “Level 2,” there was a 77.8% correlation of cell type labels, again with more abundant cell types having higher alignment while rare cell populations continued to struggle (FIG. 30).

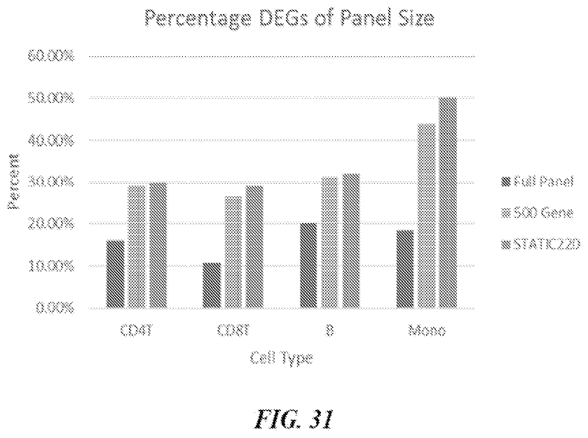

To investigate that the STATIC 220 panel is still able to capture important biological changes in the FRP chemistry, we performed a DEG analysis comparing major cell types between a stim and control condition. We found that the STATIC 220 panel was able to capture a higher number of DEGs when compared to the size of the probe set used. On average, 16.24% of the full 18,082 gene panel were DEGs, while 35.25% of the STATIC 220 genes were DEGs. The 500 gene panel fell right in between at 32.65% of the genes in the panel being DEGs (FIG. 31).

10× Genomics' new Chromium Fixed RNA Profiling (FRP) kit is going to enable near whole-transcriptome level gene expression profiling while greatly scaling the number of cells that we can capture in a single experiment, as well as reducing cost. This assay has the capabilities of becoming the main single cell assay for a number of labs, so with that in mind, we wanted to demonstrate that the STATIC 220 gene panel was still viable on this chemistry.

In the FRP panel, 200 out of the 220 genes in the STATIC gene list have probes. All 200 of these genes were detected and 86% were captured at higher average UMIs per cell when compared with the legacy 3′ assay, demonstrating that the majority of the STATIC 220 genes have an even higher sensitivity in the new chemistry.

The next thing that we wanted to test was the ability of the STATIC 220 gene panel to be able to accurately identify cell type identity when using label transfer from a reference dataset compared to having the full 18,082 probe set provided in the FRP kit. The broad “Level 1” cell types provided in the reference dataset saw a high degree of overlap at 94.8%. Only the rare cell type category “other” really struggled in this comparison, so with the addition of more cells or a larger dataset, we would probably see this cell type category increase its performance. The same held true for the “Level 2” cell type labeling. We saw an overall decrease in performance down to 77.8% with more abundant populations like CD14 monocytes and CD4 naive T cells having a 96% and 95% alignment, respectively, while very rare subsets like ILCs and Platelets had a 0% alignment. Again, with increasing number of cells in the dataset, we would expect to see better cell type label correlation between the full panel and the STATIC 220 panel.

Thus, the STATIC 220 gene panel was also able to capture transcriptional changes between a stim and control sample. From the DEG analysis, the number of differentially expressed genes averaged around 16.24% of the full 18,082 gene panel, while this increased to 35.25% in the STATIC 220 panel. So, this STATIC 220 panel was able to capture significant transcriptional differences between conditions while wasting less sequencing reads to genes that were not affected by the stim. The 500 gene panel performed more closely to the STATIC 220 than the full panel. 461 genes out of the 500 gene panel have probes in the FRP kit and on average 32.65% of the genes are DEGs in this stim experiment. Therefore, the 500 gene panel is also more efficient than using the full panel.

From the foregoing, it will be appreciated that specific embodiments of the invention have been described herein for purposes of illustration, but that various modifications may be made without deviating from the scope of the invention. Accordingly, the invention is not limited except as by the appended claims.

Lengthy table referenced here
US20250059608A1-20250220-T00001
Please refer to the end of the specification for access instructions.

Lengthy table referenced here
US20250059608A1-20250220-T00002
Please refer to the end of the specification for access instructions.

Lengthy table referenced here
US20250059608A1-20250220-T00003
Please refer to the end of the specification for access instructions.

Lengthy table referenced here
US20250059608A1-20250220-T00004
Please refer to the end of the specification for access instructions.

Lengthy table referenced here
US20250059608A1-20250220-T00005
Please refer to the end of the specification for access instructions.

Lengthy table referenced here
US20250059608A1-20250220-T00006
Please refer to the end of the specification for access instructions.

Lengthy table referenced here
US20250059608A1-20250220-T00007
Please refer to the end of the specification for access instructions.

Lengthy table referenced here
US20250059608A1-20250220-T00008
Please refer to the end of the specification for access instructions.

Lengthy table referenced here
US20250059608A1-20250220-T00009
Please refer to the end of the specification for access instructions.

Lengthy table referenced here
US20250059608A1-20250220-T00010
Please refer to the end of the specification for access instructions.

Lengthy table referenced here
US20250059608A1-20250220-T00011
Please refer to the end of the specification for access instructions.

Lengthy table referenced here
US20250059608A1-20250220-T00012
Please refer to the end of the specification for access instructions.

Lengthy table referenced here
US20250059608A1-20250220-T00013
Please refer to the end of the specification for access instructions.

Lengthy table referenced here
US20250059608A1-20250220-T00014
Please refer to the end of the specification for access instructions.

Lengthy table referenced here
US20250059608A1-20250220-T00015
Please refer to the end of the specification for access instructions.

Lengthy table referenced here
US20250059608A1-20250220-T00016
Please refer to the end of the specification for access instructions.

Lengthy table referenced here
US20250059608A1-20250220-T00017
Please refer to the end of the specification for access instructions.

Lengthy table referenced here
US20250059608A1-20250220-T00018
Please refer to the end of the specification for access instructions.

Lengthy table referenced here
US20250059608A1-20250220-T00019
Please refer to the end of the specification for access instructions.

Lengthy table referenced here
US20250059608A1-20250220-T00020
Please refer to the end of the specification for access instructions.

Lengthy table referenced here
US20250059608A1-20250220-T00021
Please refer to the end of the specification for access instructions.

Lengthy table referenced here
US20250059608A1-20250220-T00022
Please refer to the end of the specification for access instructions.

Lengthy table referenced here
US20250059608A1-20250220-T00023
Please refer to the end of the specification for access instructions.

Lengthy table referenced here
US20250059608A1-20250220-T00024
Please refer to the end of the specification for access instructions.

Lengthy table referenced here
US20250059608A1-20250220-T00025
Please refer to the end of the specification for access instructions.

Lengthy table referenced here
US20250059608A1-20250220-T00026
Please refer to the end of the specification for access instructions.

Lengthy table referenced here
US20250059608A1-20250220-T00027
Please refer to the end of the specification for access instructions.

Lengthy table referenced here
US20250059608A1-20250220-T00028
Please refer to the end of the specification for access instructions.

Lengthy table referenced here
US20250059608A1-20250220-T00029
Please refer to the end of the specification for access instructions.

Lengthy table referenced here
US20250059608A1-20250220-T00030
Please refer to the end of the specification for access instructions.

Lengthy table referenced here
US20250059608A1-20250220-T00031
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20250059608A1). An electronic copy of the table will also be available from the USPTO upon request and payment of the fee set forth in 37 CFR 1.19(b)(3).

I/We claim:

1. A method of identifying, detecting, and/or monitoring a health condition in a subject in need thereof, comprising measuring levels of a set of genes in a biological sample obtained from the subject, wherein the set of genes comprises all or a subset of A1BG, ABLIM1, AC020656.1, AC243960.1, ADTRP, AFF3, ALDH2, ANXA2R, APOBEC3C, APP, AQP3, ARID5B, ATF7IP2, BANK1, BCL11A, BCL11B, BIRC3, BLK, CAMK4, CAPG, CARS, CASP8AP2, CBL, CCDC167, CCDC50, CCL4, CCND2, CCR7, CD14, CD27, CD36, CD6, CD68, CD79A, CD79B, CD8A, CD8B, CD96, CDKN1C, CEBPD, CFD, CFP, CLEC10A, CLEC12A, CLIC3, CMC1, CPVL, CSF3R, CST7, CSTA, CTSH, CXXC5, CYBB, CYTOR, DCTPP1, DNAJB1, DOK2, DYNLL2, DYRK2, EAF2, EBP, ERN1, FCER1A, FCER1G, FCER2, FCGR3A, FCN1, FCRL1, FGFBP2, FGL2, FHIT, FKBP11, GATA3, GBP5, GIMAP7, GNG2, GPR65, GRN, GZMA, GZMB, GZMH, GZMK, HLA-DMA, HLA-DMB, HLA-DQA1, HOPX, IFITM3, IFT57, IGHD, IGHM, IGLC2, IGSF6, IKZF3, IL2RB, IL3RA, IL4R, IL6ST, INPP4B, IRF7, IRF8, ITGAL, ITM2C, JAML, JCHAIN, JUN, KLRB1, KLRC1, KLRD1, KLRF1, KLRG1, LEF1, LGALS2, LGALS3, LILRA4, LINC00623, LINC00861, LINC00926, LINC01857, LINC01871, LINC02446, LRRC25, LY86, LYN, LYST, MAL, MAML2, MAPKAPK2, MARCHF1, MARCKS, MATK, MEF2C, MHENCR, MNDA, MS4A1, MS4A6A, MS4A7, MT1X, MYBL1, MYC, MYO1F, MZB1, NCF2, NCR3, NELL2, ORAI2, OXNAD1, PAG1, PASK, PDE3B, PDLIM1, PECAM1, PHACTR2, PIK3IP1, PILRA, PITPNC1, PLD4, PLPP5, POU2AF1, POU2F2, PPP1 R10, PRF1, PRKCH, PRR5, PTPN4, PTPN6, PYHIN1, RALGPS2, RASSF1, RCAN3, RFLNB, RHOC, RNF130, RTKN2, S100A12, S100B, SAMD3, SELENOM, SERPINA1, SERPINF1, SESN3, SLC2A4RG, SLC4A10, SMIM25, SP140, SPI1, SPIB, SPON2, STMN1, STMN3, STX7, SWAP70, SYNE1, TAGAP, TBC1D15, TC2N, TCF4, TCL1A, THEM4, TMEM154, TMEM156, TMIGD2, TNFRSF13C, TNFRSF1B, TPD52, TPM2, TPST2, TRABD2A, TRDC, TRG-AS1, TRGC1, TRGC2, TSHZ2, TSPAN3, TULP4, UGCG, VCAN, XCL1, XCL2, ZAP70, and ZEB2.

2. The method of claim 1, wherein the health condition is a condition impacted by age, environmental, occupational, and/or physical factors.

3. The method of claim 1 or 2, wherein the health condition is a disease condition.

4. The method of claim 3, further comprising treating the subject for the disease condition.

5. The method of claim 4, further comprising measuring levels of the set of genes in a second biological sample obtained from the subject after the treatment.

6. The method of any one of claims 1-5, wherein the set of genes comprises about 10 or more genes, about 25 or more genes, about 50 or more genes, about 100 or more genes, about 150 or more genes, or about 200 or more genes.

7. The method of any one of claims 1-6, wherein the biological sample is a tissue sample.

8. The method of any one of claims 1-6, wherein the biological sample is a blood sample.

9. The method of claim 8, wherein the biological sample comprises peripheral blood mononuclear cells (PBMCs).

10. The method of claim 8, wherein the biological sample comprises circulating tumor cells (CTCs).

11. The method of any one of claims 1-10, wherein the measuring step is carried out by single cell technology.

12. The method of claim 11, wherein the single cell technology comprises single-cell ribonucleic acid sequencing (scRNA-seq) and/or single-cell assay for transposase-accessible chromatin sequencing (scATAC-seq).

13. The method of claim 3, wherein the disease condition is a viral infection.

14. The method of claim 13, wherein the viral infection is influenza or SARS-CoV-2 infection.

15. The method of claim 3, wherein the disease condition is cancer.

16. The method of claim 15, wherein the cancer is a hematological malignancy.

17. The method of claim 16, wherein the hematological malignancy is selected from the group consisting of monoclonal B cell lymphocytosis, multiple myeloma, myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), blast crisis chronic myelogenous leukemia (bcCML), B cell acute lymphoid leukemia (B-ALL), T cell acute lymphoid leukemia (T-ALL), T cell lymphoma, and B cell lymphoma.

18. The method of claim 15, wherein the cancer is a solid tumor.

19. The method of claim 18, wherein the solid tumor is selected from the group consisting of lung cancer, breast cancer, liver cancer, stomach cancer, colon cancer, rectal cancer, kidney cancer, gastric cancer, gallbladder cancer, cancer of the small intestine, esophageal cancer, melanoma, bone cancer, pancreatic cancer, skin cancer, uterine cancer, ovarian cancer, testicular cancer, cancer of the thyroid gland, cancer of the adrenal gland, bladder cancer, and glioma.

20. The method of claim 3, wherein the disease condition is an autoimmune disease.

21. The method of claim 20, wherein the autoimmune disease is selected from the group consisting of type 1 diabetes, lupus, systemic lupus erythematosus, rheumatoid arthritis, psoriasis, psoriatic arthritis, multiple sclerosis, inflammatory bowel disease, Crohn's disease, ulcerative colitis, Addison's disease, Graves' disease, Sjögren's syndrome, Hashimoto's thyroiditis, myasthenia gravis, autoimmune vasculitis, pernicious anemia, and celiac disease.

22. A method of identifying, labeling, and/or quantifying immune cell types in a biological sample, comprising measuring levels of a set of genes in the biological sample, wherein the set of genes comprises all or a subset of A1BG, ABLIM1, AC020656.1, AC243960.1, ADTRP, AFF3, ALDH2, ANXA2R, APOBEC3C, APP, AQP3, ARID5B, ATF7IP2, BANK1, BCL11A, BCL11B, BIRC3, BLK, CAMK4, CAPG, CARS, CASP8AP2, CBL, CCDC167, CCDC50, CCL4, CCND2, CCR7, CD14, CD27, CD36, CD6, CD68, CD79A, CD79B, CD8A, CD8B, CD96, CDKN1C, CEBPD, CFD, CFP, CLEC10A, CLEC12A, CLIC3, CMC1, CPVL, CSF3R, CST7, CSTA, CTSH, CXXC5, CYBB, CYTOR, DCTPP1, DNAJB1, DOK2, DYNLL2, DYRK2, EAF2, EBP, ERN1, FCER1A, FCER1G, FCER2, FCGR3A, FCN1, FCRL1, FGFBP2, FGL2, FHIT, FKBP11, GATA3, GBP5, GIMAP7, GNG2, GPR65, GRN, GZMA, GZMB, GZMH, GZMK, HLA-DMA, HLA-DMB, HLA-DQA1, HOPX, IFITM3, IFT57, IGHD, IGHM, IGLC2, IGSF6, IKZF3, IL2RB, IL3RA, IL4R, IL6ST, INPP4B, IRF7, IRF8, ITGAL, ITM2C, JAML, JCHAIN, JUN, KLRB1, KLRC1, KLRD1, KLRF1, KLRG1, LEF1, LGALS2, LGALS3, LILRA4, LINC00623, LINC00861, LINC00926, LINC01857, LINC01871, LINC02446, LRRC25, LY86, LYN, LYST, MAL, MAML2, MAPKAPK2, MARCHF1, MARCKS, MATK, MEF2C, MHENCR, MNDA, MS4A1, MS4A6A, MS4A7, MT1X, MYBL1, MYC, MYO1F, MZB1, NCF2, NCR3, NELL2, ORAI2, OXNAD1, PAG1, PASK, PDE3B, PDLIM1, PECAM1, PHACTR2, PIK3IP1, PILRA, PITPNC1, PLD4, PLPP5, POU2AF1, POU2F2, PPP1R10, PRF1, PRKCH, PRR5, PTPN4, PTPN6, PYHIN1, RALGPS2, RASSF1, RCAN3, RFLNB, RHOC, RNF130, RTKN2, S100A12, S100B, SAMD3, SELENOM, SERPINA1, SERPINF1, SESN3, SLC2A4RG, SLC4A10, SMIM25, SP140, SPI1, SPIB, SPON2, STMN1, STMN3, STX7, SWAP70, SYNE1, TAGAP, TBC1D15, TC2N, TCF4, TCL1A, THEM4, TMEM154, TMEM156, TMIGD2, TNFRSF13C, TNFRSF1B, TPD52, TPM2, TPST2, TRABD2A, TRDC, TRG-AS1, TRGC1, TRGC2, TSHZ2, TSPAN3, TULP4, UGCG, VCAN, XCL1, XCL2, ZAP70, and ZEB2.

23. The method of claim 22, wherein the immune cell types comprise normal immune cells and abnormal immune cells.

24. The method of claim 22, wherein the immune cell types comprise B cells, T cells, natural killer (NK) cells, monocytes, macrophages, dendritic cells (DCs), mast cells, neutrophils, eosinophils, and basophils.

25. The method of any one of claims 22-24, wherein the set of genes comprises about 10 or more genes, about 25 or more genes, about 50 or more genes, about 100 or more genes, about 150 or more genes, or about 200 or more genes.

26. The method of any one of claims 22-25, wherein the biological sample is a tissue sample.

27. The method of any one of claims 22-25, wherein the biological sample is a blood sample.

28. The method of claim 27, wherein the biological sample comprises PBMCs.

29. The method of any one of claims 22-28, wherein the measuring step is carried out by single cell technology.

30. The method of claim 29, wherein the single cell technology comprises single-cell ribonucleic acid sequencing (scRNA-seq) and/or single-cell assay for transposase-accessible chromatin sequencing (scATAC-seq).

31. A single cell assay kit comprising probes for measuring levels of a set of genes in a biological sample, wherein the set of genes comprises all or a subset of A1BG, ABLIM1, AC020656.1, AC243960.1, ADTRP, AFF3, ALDH2, ANXA2R, APOBEC3C, APP, AQP3, ARID5B, ATF7IP2, BANK1, BCL11A, BCL11B, BIRC3, BLK, CAMK4, CAPG, CARS, CASP8AP2, CBL, CCDC167, CCDC50, CCL4, CCND2, CCR7, CD14, CD27, CD36, CD6, CD68, CD79A, CD79B, CD8A, CD8B, CD96, CDKN1C, CEBPD, CFD, CFP, CLECI0A, CLECI2A, CLIC3, CMC1, CPVL, CSF3R, CST7, CSTA, CTSH, CXXC5, CYBB, CYTOR, DCTPP1, DNAJB1, DOK2, DYNLL2, DYRK2, EAF2, EBP, ERN1, FCER1A, FCER1G, FCER2, FCGR3A, FCN1, FCRL1, FGFBP2, FGL2, FHIT, FKBP11, GATA3, GBP5, GIMAP7, GNG2, GPR65, GRN, GZMA, GZMB, GZMH, GZMK, HLA-DMA, HLA-DMB, HLA-DQA1, HOPX, IFITM3, IFT57, IGHD, IGHM, IGLC2, IGSF6, IKZF3, IL2RB, IL3RA, IL4R, IL6ST, INPP4B, IRF7, IRF8, ITGAL, ITM2C, JAML, JCHAIN, JUN, KLRB1, KLRC1, KLRD1, KLRF1, KLRG1, LEF1, LGALS2, LGALS3, LILRA4, LINC00623, LINC00861, LINC00926, LINC01857, LINC01871, LINC02446, LRRC25, LY86, LYN, LYST, MAL, MAML2, MAPKAPK2, MARCHF1, MARCKS, MATK, MEF2C, MHENCR, MNDA, MS4A1, MS4A6A, MS4A7, MT1X, MYBL1, MYC, MYO1F, MZB1, NCF2, NCR3, NELL2, ORAI2, OXNAD1, PAG1, PASK, PDE3B, PDLIM1, PECAM1, PHACTR2, PIK3IP1, PILRA, PITPNC1, PLD4, PLPP5, POU2AF1, POU2F2, PPP1R10, PRF1, PRKCH, PRR5, PTPN4, PTPN6, PYHIN1, RALGPS2, RASSF1, RCAN3, RFLNB, RHOC, RNF130, RTKN2, S100A12, S100B, SAMD3, SELENOM, SERPINA1, SERPINF1, SESN3, SLC2A4RG, SLC4A10, SMIM25, SP140, SPI1, SPIB, SPON2, STMN1, STMN3, STX7, SWAP70, SYNE1, TAGAP, TBC1D15, TC2N, TCF4, TCL1A, THEM4, TMEM154, TMEM156, TMIGD2, TNFRSF13C, TNFRSF1B, TPD52, TPM2, TPST2, TRABD2A, TRDC, TRG-AS1, TRGC1, TRGC2, TSHZ2, TSPAN3, TULP4, UGCG, VCAN, XCL1, XCL2, ZAP70, and ZEB2.

32. The single cell assay kit of claim 31, wherein the set of genes comprises about 10 or more genes, about 25 or more genes, about 50 or more genes, about 100 or more genes, about 150 or more genes, or about 200 or more genes.

33. The single cell assay kit of claim 31 or 32, wherein the biological sample is a tissue sample.

34. The single cell assay kit of claim 31 or 32, wherein the biological sample is a blood sample.

35. The single cell assay kit of claim 34, wherein the biological sample comprises PBMCs.

36. The single cell assay kit of any one of claims 31-35, wherein the single cell assay comprises single-cell ribonucleic acid sequencing (scRNA-seq) and/or single-cell assay for transposase-accessible chromatin sequencing (scATAC-seq).
